(19) United States
(12) Patent Application Publication
(10) Pub. No.: US 2024/0092882 A1
(43) Pub. Date: Mar. 21, 2024

(54) METHOD OF TREATING AN ALLERGY WITH ALLERGEN-SPECIFIC MONOCLONAL ANTIBODIES

(71) Applicant: REGENERON PHARMACEUTICALS, INC., Tarrytown, NY (US)

(72) Inventors: Meagan O'BRIEN, New York, NY (US); Gary HERMAN, Princeton, NJ (US); Allen RADIN, New York, NY (US); Michelle DEVEAUX, White Plains, NY (US); Lorah PERLEE, Wilton, CT (US); Jamie M. ORENGO, Cortlandt Manor, NY (US)

(21) Appl. No.: 18/462,320

(22) Filed: Sep. 6, 2023

Related U.S. Application Data
(63) Continuation of application No. 17/411,979, filed on Aug. 25, 2021.
(60) Provisional application No. 63/070,417, filed on Aug. 26, 2020.
(60) Provisional application No. 63/153,243, filed on Feb. 24, 2021.
(60) Provisional application No. 63/212,532, filed on Jun. 18, 2021.

(51) Int. Cl. C07K 16/18; A61P 37/08

(52) U.S. Cl. CPC C07K 16/18; A61P 37/08; A61K 2039/507

(57) ABSTRACT

The present invention provides methods for treating an allergy by administering one or more antibodies that bind specifically to the allergen. In certain embodiments, the antibodies useful for treating an allergen, bind specifically to the cat allergen, Fel d1. According to certain embodiments of the invention, the antibodies are fully human antibodies that bind to Fel d1. The antibodies of the invention are useful for binding to the Fel d1 allergen in vivo, thus preventing binding of the Fel d1 allergen to pre-formed IgE on the surface of mast cells or basophils. In doing so, the antibodies act to prevent the release of histamine and other inflammatory mediators from mast cells and/or basophils, thus ameliorating the untoward response to the cat allergen in sensitized individuals.

### CROSS-REFERENCE TO RELATED APPLICATIONS

This application is a continuation of U.S. patent application Ser. No. 17/411,979, filed on Aug. 25, 2021, which claims priority to U.S. Provisional Patent Application Nos. 63/070,417, filed on Aug. 26, 2020, 63/153,243, filed on Feb. 24, 2021, and 63/212,532, filed on Jun. 18, 2021, the entire content of each of which is incorporated by reference herein.

### FIELD OF THE INVENTION

The present invention is related to monoclonal antibodies and antigen-binding fragments thereof that bind specifically to an allergen for use in treating patients who demonstrate a sensitivity to, or an allergic reaction against an allergen.

### SEQUENCE STATEMENT

The instant application contains a Sequence Listing, which has been submitted electronically in XML format and is hereby incorporated by reference in its entirety. Said XML file, created on Sep. 5, 2023, is named 40848_0106 USC1_SL.xml and is 419,137 bytes in size.

### STATEMENT OF RELATED ART

Cat allergens are among the most important indoor allergens and a common cause of Type 1 (IgE-mediated) allergic disease worldwide, affecting 10 to 15% of patients with allergic rhinoconjunctivitis and/or asthma. Felis catus (domestic cat) allergen 1 (Fel d1) in cat hair is produced by the skin and by salivary and lacrimal glands of the cat (Kleine-Tebbe, et al., 1993 Int Arch Allergy Immunol 100(3):256-62); Gronlund, et al., 2010 Int Arch Allergy Immunol 151(4):265-74). Dried saliva and dandruff are spread from the cat hair as small airborne particles into the surrounding environment that readily adhere to surfaces such as walls, carpets, and furniture. While the highest amount of Fel d1 allergen is found in households with cats and the concentration correlates with the number of cats kept in a home (Bollinger, et al., 1996 J Allergy Clin Immunol 97:907-14; Bollinger, et al., 1998 J Allergy Clin Immunol 101:124-5; Neisler, et al., 2016 Aerobiologia 32:571-580), this allergen can also be carried on clothes and shoes into homes and schools without cats and may persist in these areas for months to years (Karlsson, A., 2004 Allergy 59: 661-667). Therefore, it is difficult to avoid exposure to cat hair allergen in the environment.

Nasal and eye symptoms are the most frequently reported and most bothersome of all cat allergy symptoms. Rhinoconjunctivitis is treated with antihistamines and intranasal corticosteroids (INS), which are only moderately effective for nasal symptoms (Ciprandi, et al., 2011 Curr Med Res Opin 27(5) 1005-11). The best reported treatment effects for antihistamines and INS are 5 to 11% relative reduction of total nasal symptoms compared to placebo (Durham, et al., 2016 J. Allergy Clin. Immunol. 138 (4) 1081-1088). Intranasal corticosteroids are considered ineffective for allergic eye symptoms.

The association between cat allergy and asthma is significant. Approximately 30% of allergic asthmatics reportedly have a concomitant allergy to cats (Arbes, et al., 2007 J Allergy Clin Immunol 120(5) 1139-45). More than 50% of cat sensitized patients have a diagnosis of co-morbid asthma, ranging from intermittent mild to potentially life-threatening asthmatic exacerbations requiring treatment with short- and long acting bronchodilators, inhaled corticosteroids, and broader immune-targeting agents (Giavina-Bianchi, et al., 2016 J Asthma and Allergy 9:93-100). Patients with high concentrations of cat allergen-specific IgE are at higher risk for oculo-nasal and/or asthma symptoms, or both (Perzanowski, et al., 2016 J Allergy Clin Immunol 138(6):1582-1590; Olivieri, M., 2016 Allergy 71: 859-868).

Specific immunotherapy (SIT) is a disease-modifying, standard-of-care treatment option for patients with allergic rhinoconjunctivitis triggered by cat allergen when pharmacological therapies are insufficient (Zuberbier, et al., 2010 Allergy 65(12):1525-30; Walker, et al., 2011 Clin Exp Allergy 41(9):1177-2000). Allergen-specific polyclonal IgG4 titers increase during SIT and may inhibit effector cell activation by blocking binding of IgE-allergen complex to high-affinity IgE receptors on mast cell and basophil surfaces, thereby effectively preventing early phase allergic symptoms (Kundig, et al., 2010 Human Vaccines 6:8, 673-675; James, et al., 2011 J Allergy Clin Immunol 127: 509-16, e5). Clinical symptom improvement correlates with the ability of blocking IgG4s to compete with IgE for allergen binding. Although SIT can provide long-lasting protection from allergic disease, SIT carries a risk of local and systemic adverse reactions (especially in uncontrolled or severe asthma), is variably effective among different patients, and can take 3 to 5 years to induce permanent immune tolerance (Leung, et al., 2010 Pediatric Allergy: Principals and Practice; St. Louis: Mosby; Durham, et al., 2012 J. Allergy Clin Immunol 137(2):339-349.e10; Scadding, et al., 2017 JAMA 317(6):615-625). As such, there is a need in the art for a more rapid and reliable approach to treating allergies.

REGN1908 and REGN1909 are monoclonal antibodies (mAbs), which bind independently and non-competitively to the Fel d1 allergen and are being developed as a cocktail (REGN1908-1909) for the treatment of allergic disease triggered by exposure to cats or cat hair. REGN1908 and REGN1909 are described in WO2018/118713A1 and U.S. Pat. No. 9,079,948. Fel d1 is the major cat allergen that is recognized in more than 90% of cat-allergic patients (van Ree, et al., 1999 J Allergy Clin Immunol 104(6):1223-30) and accounts for 60-90% of the total allergenic activity in cat dander (Kleine-Tebbe, 1993). These fully human, high-affinity IgG4 monoclonal antibodies targeted to the major cat allergen are designed to block Fel d1 from binding to IgE, as well as or better than those naturally produced during SIT (Orengo, et al., 2018 (in press) Nature Communications).

### BRIEF SUMMARY OF THE INVENTION

In its broadest aspect, the invention provides methods of treating a patient who demonstrates a sensitivity to, or an allergic reaction against an allergen, or for preventing or ameliorating at least one symptom or complication associated with the allergen by administering one or more antibodies or antigen binding fragments thereof specific for the allergen (passive immunotherapy). The results described herein show that antibodies specific for an allergen, when administered to a patient having an allergy, can prevent an allergen-induced allergic response. As such, it is believed that a passive immunotherapy approach such as that described herein, can be used in place of standard immunotherapy (SIT), which generally takes years of desensitization to provide effective relief to the patient, whereas positive results can be achieved in a matter of weeks by administering allergen-specific antibodies or antigen binding fragments thereof.

In one aspect, the invention provides a method for reducing the severity, duration, or frequency of occurrence of one or more symptoms associated with an allergic response in a patient, wherein the allergic response is the result of exposure to an animal product, for example, a cat allergen, the method comprising administering one or more therapeutically effective doses of at least two allergen-specific monoclonal antibodies or antigen binding fragments thereof to the patient.

In certain embodiments of the methods according to the invention, the one or more symptoms are selected from the group consisting of early asthmatic response (EAR), acute bronchoconstriction, allergic rhinitis, conjunctivitis, chest tightness, shortness of breath, wheezing, coughing, nasal congestion, nasal itching, rhinorrhea, sneezing, and ocular symptoms.

In another aspect, the invention provides a method for increasing the time to early asthmatic response (EAR) upon exposure to an animal product, for example, a cat allergen, in a patient having an allergy to the cat allergen, the method comprising administering one or more therapeutically effective doses of at least two allergen-specific monoclonal antibodies or antigen binding fragments thereof to the patient.

In another aspect, the invention provides a method for increasing the time to early asthmatic response (EAR) upon exposure to a cat allergen in a patient having an allergy to the cat allergen, the method comprising administering one or more therapeutically effective doses of at least two allergen-specific monoclonal antibodies or antigen binding fragments thereof to the patient. In certain embodiments of the methods according to the invention, the cat allergen is Fel d1. In further embodiments, the incidence of EAR is reduced by about 20% to about 50% after administration of the one or more therapeutically effective doses of at least two allergen-specific monoclonal antibodies or antigen binding fragments thereof to the patient. In further embodiments, the incidence of EAR is reduced for at least 56 days after administration of the one or more therapeutically effective doses of at least two allergen-specific monoclonal antibodies or antigen binding fragments thereof to the patient. In still further embodiments, patient-reported chest tightness is reduced from baseline. In still further embodiments, patient-reported breathing difficulty is reduced from baseline. In still further embodiments, the patient has a reduced likelihood of needing rescue medication upon exposure to the allergen for up to at least about three months after administration of the one or more therapeutically effective doses of at least two allergen-specific monoclonal antibodies or antigen binding fragments thereof. The reduced likelihood is measured as a proportion of patients needing rescue medication. In further embodiments, the time to EAR is increased to greater than 4 hours.

In another embodiment, the instantaneous probability that the patient experiences an EAR during exposure to the animal product is reduced by at least 50%. In still another embodiment, the instantaneous probability that the patient experiences an EAR during exposure to the animal product is reduced by about 64%. “Instantaneous probability” of experiencing an EAR during exposure, as used herein, refers to the probability of experiencing an EAR at any particular point in time during exposure to the allergen.

In another aspect, the invention provides a method for increasing the quantity of an animal product tolerated by a patient having an allergy to the animal product, the method comprising administering one or more therapeutically effective doses of at least two allergen-specific monoclonal antibodies or antigen binding fragments thereof to the patient. In one embodiment, the quantity of the animal product tolerated by the patient is increased by at least about 300%.

In another aspect, the invention provides a method for improving lung function, and/or for reducing bronchoconstriction, upon exposure to a cat allergen in a patient having an allergy to the cat allergen, the method comprising administering one or more therapeutically effective doses of at least two allergen-specific monoclonal antibodies or antigen binding fragments thereof to the patient. In one embodiment, the improvement in lung function comprises an increase in the forced expiratory volume (FEV1) area under curve (AUC) of the patient. In another embodiment, the increase in FEV1 AUC is at least about 12%.

In one embodiment of the methods according to the invention, the patient has a cat allergy. In another embodiment, the patient has mild asthma. In still another embodiment, the patient has conjunctivitis. In another embodiment the patient has allergic rhinitis.

In one embodiment of the methods according to the invention, the animal product contains the allergen Fel d1.

In another embodiment of the methods according to the invention, the at least two allergen-specific monoclonal antibodies or antigen-binding fragments thereof bind specifically to Fel d1 protein. In another embodiment, the at least two allergen-specific monoclonal antibodies or antigen-binding fragments thereof that bind specifically to Fel d1 protein comprise the three heavy chain CDRs (HCDR1, HCDR2 and HCDR3) contained within the heavy chain variable region (HCVR) sequences of SEQ ID NOs: 18 and 306, respectively; and the three light chain CDRs (LCDR1, LCDR2 and LCDR3) contained within the light chain variable region (LCVR) sequences of SEQ ID NOs: 26 and 314, respectively. In still another embodiment, the at least two antibodies that bind specifically to Fel d1 protein comprise a HCVR having an amino acid sequence of SEQ ID NOs: 18 and 306, respectively. In still another embodiment, the at least two antibodies that bind specifically to Fel d1 protein comprise a LCVR having an amino acid sequence of SEQ ID NOs: 26 and 314, respectively. In still another embodiment, the at least two antibodies that bind specifically to Fel d1 protein comprise a HCVR/LCVR amino acid sequence pair of SEQ ID NOs: 18/26 and 306/314, respectively. In still another embodiment, the at least two antibodies that bind specifically to Fel d1 protein comprise: a HCDR1 domain having an amino acid sequence of SEQ ID NOs: 20 and 308, respectively; a HCDR2 domain having an amino acid sequence of SEQ ID NOs: 22 and 310, respectively; a HCDR3 domain having an amino acid sequence of SEQ ID NOs: 24 and 312, respectively; a LCDR1 domain having an amino acid sequence of SEQ ID NOs: 28 and 316, respectively; a LCDR2 domain having an amino acid sequence of AAS and KAS, respectively; and a LCDR3 domain having an amino acid sequence of SEQ ID NOs: 32 and 320, respectively.

In one embodiment of the methods according to the invention, the at least two allergen-specific antibodies are administered subcutaneously, intravenously, or intranasally to the patient. In one embodiment, the at least two allergen-specific monoclonal antibodies are administered to the patient therapeutically or prophylactically. In one embodiment, the at least two allergen-specific monoclonal antibodies are formulated individually or are co-formulated for administration to the patient.

In another embodiment of the methods according to the invention, the at least two allergen-specific antibodies are administered sequentially or concurrently to the subject. In a further embodiment, the at least two allergen-specific antibodies are administered concurrently to the subject.

In one embodiment of the methods according to the invention, the at least two allergen-specific antibodies are administered subcutaneously, each at a dose of about 5 mg to about 400 mg. In another embodiment, the at least two allergen-specific antibodies are administered subcutaneously, each at a dose of about 300 mg. In one embodiment, the total amount of the two allergen-specific monoclonal antibodies to be administered to the patient is 600 mg (300 mg of each antibody).

In one embodiment of the methods according to the invention, one therapeutically effective dose of at least two allergen-specific monoclonal antibodies or antigen binding fragments thereof is administered to the patient. In another embodiment of the methods according to the invention, one therapeutically effective dose of two allergen-specific monoclonal antibodies or antigen-binding fragments thereof is administered to the patient.

While it is believed that the therapeutic approach described herein may be effective using one or more antibodies or antigen binding fragments thereof to any allergen, the proof-of-concept studies described herein were conducted using two fully human monoclonal antibodies (mAbs) or antigen-binding fragments thereof that bind specifically to the cat allergen, Fel d1.

The antibodies of the invention can be full-length (for example, an IgG1 or IgG4 antibody) or may comprise only an antigen-binding portion (for example, a Fab, F(ab′)2 or scFv fragment), and may be modified to affect functionality, e.g., to eliminate residual effector functions (Reddy, et al., (2000), J. Immunol. 164:1925-1933).

In yet other embodiments, a patient is monitored before and after administration of the one or more allergen specific antibodies or antigen binding fragments thereof to determine efficacy of the treatment. In additional embodiments, a method comprises administering one or more therapeutically effective doses of one or more allergen-specific antibodies or antigen binding fragments thereof to the patient and measuring one or more of the time to EAR, FEV1 AUC, amount of allergen tolerated, nasal symptoms, and ocular symptoms in a patient. In embodiments, a measurement is conducted to determine a baseline prior to administration and then at various timepoints post administration in order to determine the efficacy of the therapy.

In any of the methods described herein, the allergen may be selected from an animal product, a food allergen, plant pollen, mold spores, house dust mites, cockroaches, perfume, detergents, household cleaners, latex, a drug product, or insect venom.

The animal product may be selected from the group consisting of animal fur, animal dander, wool, and mite excretions.

The animal product may be selected from the group consisting of cat dander, cat hair or an extract thereof, or to the Fel d1 protein.

In certain embodiments, the animal product may contain the allergen can f1, can f2, can f3, can f4, can f5 or can f6.

In one embodiment, the food allergen may be selected from the group consisting of eggs, meat, fruit, legumes, milk or other dairy products, seafood, sesame, soy, wheat, oat, barley, celery and celeriac, corn or maize and tree nuts. The legumes may be selected from the group consisting of peanuts, beans, peas and soybeans. The tree nuts may be selected from the group consisting of pecans, almonds, cashews, hazelnuts (filberts), walnuts, brazil nuts, macadamia nuts, chestnuts, pine nuts and pistachio nuts.

In one embodiment, the allergen may be selected from the group consisting of grass pollen, weed pollen, and tree pollen. The tree pollen may be selected from the group consisting of birch pollen, cedar pollen, oak pollen, alder pollen, hornbeam pollen, aesculus pollen, willow pollen, poplar pollen, plantanus pollen, tilia pollen, olea pollen, Ashe juniper pollen, and Alstonia scholaris pollen.

In one embodiment, the birch pollen contains the allergen Betv 1.

In one embodiment, the cedar pollen contains the allergen Cryj1 or Cryj2.

In one embodiment, the grass pollen is ryegrass or timothy-grass.

In one embodiment, the weed pollen is selected from the group consisting of ragweed, plantago, nettle, Artemisia vulgaris, Chenopodium album and sorrel.

In one embodiment, the insect venom is produced by bees, wasps or fire ants.

In certain embodiments, the methods further comprise administering an effective amount of a second therapeutic agent useful for diminishing an allergic reaction to an allergen, wherein the second therapeutic agent is selected from the group consisting of a bronchial dilator, an antihistamine, epinephrine, a decongestant, a corticosteroid, an IL-4R antagonist, an anti-IgE antibody, or one or more different antibodies to the allergen.

In one embodiment, when a patient is screened to select one or more allergen specific antibodies, the determining step is carried out by an in vitro method selected from the group consisting of an enzyme-linked immunosorbent assay (ELISA), a radioimmunoassay (RIA), an immunoradiometric assay (IRMA), a luminescence immunoassay (LIA), an immunoblot, FACS analysis, an IgE-facilitated allergen binding (FAB) assay, a basophil activation assay (e.g., a functional phosphoflow assay, or a basophil activation test), and an assay using an engineered cell line expressing FCcR1. In other embodiments, the determining step is carried out in vivo using an allergen-specific animal model such as a passive cutaneous anaphylaxis model (PCA). In embodiments, both an in vitro and in vivo assay can be utilized.

In certain embodiments, the method further comprises administering a palliative therapy useful for reducing the severity of the allergic reaction or for ameliorating at least one symptom associated with the allergic reaction.

In one embodiment, the treating results in a lessening in severity and/or duration of at least one symptom or complication associated with the allergic reaction against the allergen, wherein the one symptom or complication associated with the allergic reaction is selected from the group consisting of sneezing, rhinorrhea, nasal itching and nasal congestion.

In one embodiment, the treating results in a reduction in allergic rhinitis, allergic conjunctivitis, rhinoconjunctivitis, allergic asthma, asthma exacerbations, or an anaphylactic response following exposure of the patient to an allergen.

In any of the methods described herein, the method provides for administering one or more antibodies that bind specifically to the allergen, e.g., Fel d1 protein.

In one embodiment, the at least one isolated human antibody or antigen-binding fragment thereof that binds specifically to Fel d1 has an isotype selected from the group consisting of an IgG1, an IgG2 and an IgG4.

In one embodiment, the at least one isolated human antibody or antigen-binding fragment thereof binds specifically to Fel d1 with a K D equal to or less than 10⁻⁶ M. In one embodiment, the isolated human antibody or antigen-binding fragment thereof binds specifically to Fel d1 with a K D equal to or less than 1.8 nM.

In some embodiments, the antibodies or antigen binding fragments thereof specifically bind monomeric Fel d1 with a K D equal to or less than 1×10⁻⁸ M, 1×10⁻⁹ M, or 1×10⁻¹⁰ M. In some embodiments, the antibodies or antigen binding fragments thereof specifically bind dimeric Fel d1 with a K D equal to or less than 1×10⁻⁸ M, 1×10⁻⁹ M, 1×10⁻¹⁰ M, or 1×10⁻¹¹ M. In embodiments, the antibodies or antigen binding fragments thereof specifically bind dimeric Fel d1 with a T½ of at least 150 min, 160 min, 170 min, 180 min, 190 min, 200 min, 210 min, 220 min, 230 min, 240 min, or 250 min. In embodiments, the antibodies or antigen binding fragments thereof specifically bind monomeric Fel d1 with a T½ of at least 25 min, 30 min, 35 min, 40 min, 45 min, or 50 min.

In other embodiments, the antibodies or antigen binding fragments thereof block binding of the allergen to allergen specific IgE. In embodiments, one or more antibodies or antigen binding fragments thereof that block Fel d1-induced basophil activation are selected that have an IC50 of about 10 pM, 20 pM, 30 pM, 40 pM, 50 pM, 100 pM, 1 nM, or less. In an ELISA assay, one or more antibodies or antigen binding fragments thereof that block Fel d1 binding to polyclonal IgE are selected that have an IC50 of about 600 pM or less. One or more antibodies or antigen binding fragments thereof that block Fel d1 binding to patient IgE are selected that have an IC50 of about 500 pM or less.

In any of the methods described herein, the at least one isolated human antibody or antigen-binding fragment thereof that binds specifically to Fel d1 comprises the three heavy chain CDRs (HCDR1, HCDR2 and HCDR3) contained within any one of the heavy chain variable region (HCVR) sequences selected from the group consisting of SEQ ID NOs: 2, 18, 34, 50, 66, 82, 98, 114, 130, 146, 162, 178, 194, 210, 226, 242, 258, 274, 290, 306, 322, 338, 354, 370 and 460; and the three light chain CDRs (LCDR1, LCDR2 and LCDR3) contained within any one of the light chain variable region (LCVR) sequences selected from the group consisting of SEQ ID NOs: 10, 26, 42, 58, 74, 90, 106, 122, 138, 154, 170, 186, 202, 218, 234, 250, 266, 282, 298, 314, 330, 346, 362, 378 and 468. Methods and techniques for identifying CDRs within HCVR and LCVR amino acid sequences are well known in the art and can be used to identify CDRs within the specified HCVR and/or LCVR amino acid sequences disclosed herein. Exemplary conventions that can be used to identify the boundaries of CDRs include, e.g., the Kabat definition, the Chothia definition, and the AbM definition. In general terms, the Kabat definition is based on sequence variability, the Chothia definition is based on the location of the structural loop regions, and the AbM definition is a compromise between the Kabat and Chothia approaches. See, e.g., Kabat, “Sequences of Proteins of Immunological Interest,” National Institutes of Health, Bethesda, Md. (1991); Al-Lazikani, et al., (1997), J. Mol. Biol. 273:927-948; and Martin, et al., (1989), Proc. Natl. Acad. Sci. USA 86:9268-9272. Public databases are also available for identifying CDR sequences within an antibody.

In one embodiment, the at least one isolated human antibody or antigen-binding fragment thereof that binds specifically to Fel d1 comprises the three heavy chain CDRs (HCDR1, HCDR2 and HCDR3) contained within any one of the heavy chain variable region (HCVR) sequences selected from the group consisting of SEQ ID NOs: 18, 66, 130, 162, 242, 306, 322, 370 and 460; and the three light chain CDRs (LCDR1, LCDR2 and LCDR3) contained within any one of the light chain variable region (LCVR) sequences selected from the group consisting of SEQ ID NOs: 26, 74, 138, 170, 250, 314, 330, 378 and 468.

In one embodiment, the at least one isolated human antibody or antigen-binding fragment thereof that binds specifically to Fel d1 comprises a HCVR having an amino acid sequence selected from the group consisting of SEQ ID NOs: 2, 18, 34, 50, 66, 82, 98, 114, 130, 146, 162, 178, 194, 210, 226, 242, 258, 274, 290, 306, 322, 338, 354, 370 and 460.

In one embodiment, the at least one isolated human antibody or antigen-binding fragment thereof that binds specifically to Fel d1 comprises a HCVR having an amino acid sequence selected from the group consisting of SEQ ID NOs: 18, 66, 130, 162, 242, 306, 322, 370 and 460.

In one embodiment, the at least one isolated human antibody or antigen-binding fragment thereof that binds specifically to Fel d1 comprises a LCVR having an amino acid sequence selected from the group consisting of SEQ ID NOs: 10, 26, 42, 58, 74, 90, 106, 122, 138, 154, 170, 186, 202, 218, 234, 250, 266, 282, 298, 314, 330, 346, 362, 378 and 468.

In one embodiment, the at least one isolated human antibody or antigen-binding fragment thereof that binds specifically to Fel d1 comprises a LCVR having an amino acid sequence selected from the group consisting of SEQ ID NOs: 26, 74, 138, 170, 250, 314, 330, 378 and 468.

In one embodiment, the at least one isolated human antibody or antigen-binding fragment thereof that binds specifically to Fel d1 comprises: (a) a HCVR having an amino acid sequence selected from the group consisting of SEQ ID NOs: 2, 18, 34, 50, 66, 82, 98, 114, 130, 146, 162, 178, 194, 210, 226, 242, 258, 274, 290, 306, 322, 338, 354, 370 and 460; and (b) a LCVR having an amino acid sequence selected from the group consisting of SEQ ID NO: 10, 26, 42, 58, 74, 90, 106, 122, 138, 154, 170, 186, 202, 218, 234, 250, 266, 282, 298, 314, 330, 346, 362, 378 and 468.

In one embodiment, the at least one isolated human antibody or antigen-binding fragment thereof that binds specifically to Fel d1 comprises: (a) a HCVR having an amino acid sequence selected from the group consisting of SEQ ID NOs: 18, 66, 130, 162, 242, 306, 322, 370 and 460; and (b) a LCVR having an amino acid sequence selected from the group consisting of SEQ ID NO: 26, 74, 138, 170, 250, 314, 330, 378 and 468.

In one embodiment, the at least one isolated human antibody or antigen-binding fragment thereof that binds specifically to Fel d1 comprises:
(a) a HCDR1 domain having an amino acid sequence selected from the group consisting of SEQ ID NOs: 4, 20, 36, 52, 68, 84, 100, 116, 132, 148, 164, 180, 196, 212, 228, 244, 260, 276, 292, 308, 324, 340, 356, 372 and 462;
(b) a HCDR2 domain having an amino acid sequence selected from the group consisting of SEQ ID NOs: 6, 22, 38, 54, 70, 86, 102, 118, 134, 150, 166, 182, 198, 214, 230, 246, 262, 278, 294, 310, 326, 342, 358, 374 and 464;
(c) a HCDR3 domain having an amino acid sequence selected from the group consisting of SEQ ID NOs: 8, 24, 40, 56, 72, 88, 104, 120, 136, 152, 168, 184, 200, 216, 232,248, 264, 280, 296, 312, 328, 344, 360, 376 and 466;
(d) a LCDR1 domain having an amino acid sequence selected from the group consisting of SEQ ID NOs: 12, 28, 44, 60, 76, 92, 108, 124, 140, 156, 172, 188, 204, 220, 236, 252, 268, 284, 300, 316, 332, 348, 364, 380 and 470;
(e) a LCDR2 domain having an amino acid sequence selected from the group consisting of AAS, AAS, WAS, AAS, VVTS, KAS, DAS, KTS, DAS, RAS, AAS, GSS, YAS, YAS, AAS, YAS, YAS, AAS, YAS, KAS, GAS, TSS, AAS, SAS, and KAS; and
(f) a LCDR3 domain having an amino acid sequence selected from the group consisting of SEQ ID NOs: 16, 32, 48, 64, 80, 96, 112, 128, 144, 160, 176, 192, 208, 224, 240, 256, 272, 288, 304, 320, 336, 352, 368, 384 and 474.

In one embodiment, the at least one isolated human antibody or antigen-binding fragment thereof that binds specifically to Fel d1 comprises:
(a) a HCDR1 domain having an amino acid sequence selected from the group consisting of SEQ ID NOs: 20, 68, 132, 164, 244,308, 324, 372 and 462;
(b) a HCDR2 domain having an amino acid sequence selected from the group consisting of SEQ ID NOs: 22, 70, 134, 166, 246, 310, 326, 374 and 464;
(c) a HCDR3 domain having an amino acid sequence selected from the group consisting of SEQ ID NOs: 24, 72, 136, 168, 248, 312, 328, 376 and 466;
(d) a LCDR1 domain having an amino acid sequence selected from the group consisting of SEQ ID NOs: 28, 76, 140, 172, 252, 316, 332, 380 and 470;
(e) a LCDR2 domain having an amino acid sequence selected from the group consisting of AAS, VVTS, DAS, AAS, YAS, KAS, GAS, SAS, and KAS; and
(f) a LCDR3 domain having an amino acid sequence selected from the group consisting of SEQ ID NOs: 32, 80, 144, 176, 256, 320, 336, 384 and 474.

In one embodiment, the at least one isolated human antibody or antigen-binding fragment thereof that binds specifically to Fel d1 comprises a HCVR/LCVR amino acid sequence pair selected from the group consisting of SEQ ID NOs: 2/10, 18/26, 34/42, 50/58, 66/74, 82/90, 98/106, 114/122, 130/138, 146/154, 162/170, 178/186, 194/202, 210/218, 226/234, 242/250, 258/266, 274/282, 290/298, 306/314, 322/330, 338/346, 354/362, 370/378 and 460/468.

In one embodiment, the at least one isolated human antibody or antigen-binding fragment thereof that binds specifically to Fel d1 comprises a HCVR/LCVR amino acid sequence pair selected from the group consisting of SEQ ID NOs: 18/26, 66/74, 130/138, 162/170, 242/250, 306/314, 322/330, 370/378 and 460/468.

In one embodiment, the at least one isolated human antibody or antigen-binding fragment thereof that binds specifically to Fel d1 comprises the HCVR/LCVR amino acid sequence pair selected from the group consisting of SEQ ID NOs: 18/26 and 306/314.

In one embodiment, the at least one isolated human antibody or antigen-binding fragment thereof that binds specifically to Fel d1 interacts with at least one amino acid sequence selected from the group consisting of amino acid residues ranging from about position 15 to about position 24 of SEQ ID NO: 396; amino acid residues ranging from about position 85 to about position 103 of SEQ ID NO: 396; amino acid residues ranging from about position 85 to about position 104 of SEQ ID NO: 396; amino acid residues ranging from about position 113 to about position 116 of SEQ ID NO: 396; amino acid residues ranging from about position 113 to about position 127 of SEQ ID NO: 396; and amino acid residues ranging from about position 128 to 141 of SEQ ID NO: 396. In certain embodiments, the epitopes to which the anti-Fel d1 antibodies bind are identified using hydrogen deuterium exchange (HDX).

In one embodiment, the at least one isolated human antibody or antigen-binding fragment thereof that binds specifically to Fel d1 interacts with amino acid residues ranging from about position 15 to about position 24 of SEQ ID NO: 396.

In one embodiment, the at least one isolated human antibody or antigen-binding fragment thereof that binds specifically to Fel d1 interacts with amino acid residues ranging from about position 85 to about position 103 of SEQ ID NO: 396.

In one embodiment, the at least one isolated human antibody or antigen-binding fragment thereof that binds specifically to Fel d1 interacts with amino acid residues ranging from about position 85 to about position 104 of SEQ ID NO: 396.

In one embodiment, the at least one isolated human antibody or antigen-binding fragment thereof that binds specifically to Fel d1 interacts with amino acid residues ranging from about position 113 to about position 116 of SEQ ID NO: 396.

In one embodiment, the at least one isolated human antibody or antigen-binding fragment thereof that binds specifically to Fel d1 interacts with at least one amino acid sequence selected from the group consisting of SEQ ID NO: 402, 403, 404, 406 and 412.

In one embodiment, the at least one isolated human antibody or antigen-binding fragment thereof that binds specifically to Fel d1 interacts with SEQ ID NO: 402.

In one embodiment, the at least one isolated human antibody or antigen-binding fragment thereof that binds specifically to Fel d1 interacts with SEQ ID NO: 403.

In one embodiment, the at least one isolated human antibody or antigen-binding fragment thereof that binds specifically to Fel d1 interacts with SEQ ID NO: 404.

In one embodiment, the at least one isolated human antibody or antigen-binding fragment thereof that binds specifically to Fel d1 interacts with SEQ ID NO: 406.

In one embodiment, the at least one isolated human antibody or antigen-binding fragment thereof that binds specifically to Fel d1 interacts with SEQ ID NO: 412.

In one embodiment, the at least one isolated human antibody or antigen-binding fragment thereof that binds specifically to Fel d1 interacts with SEQ ID NO: 426.

In one embodiment, the at least one isolated human antibody or antigen binding fragment thereof that interacts with SEQ ID NOs: 402, 403, 404, 406 and/or 426, comprises the three HCDRs contained in the heavy chain variable region of SEQ ID NO: 18 and the three LCDRs contained in the light chain variable region of SEQ ID NO: 26.

In one embodiment, the at least one isolated human antibody or antigen binding fragment thereof that interacts with SEQ ID NOs: 402, 403, 404, 406 and/or 426, comprises a HCDR1 of SEQ ID NO: 20; a HCDR2 of SEQ ID NO: 22; a HCDR3 of SEQ ID NO: 24; a LCDR1 of SEQ ID NO: 28; a LCDR2 of AAS and a LCDR3 of SEQ ID NO: 32.

In one embodiment, the at least one isolated human antibody or antigen binding fragment thereof that interacts with SEQ ID NO: 412 comprises the three HCDRs contained in the heavy chain variable region of SEQ ID NO: 306 and the three LCDRs contained in the light chain variable region of SEQ ID NO: 314.

In one embodiment, the at least one isolated human antibody or antigen binding fragment thereof that interacts with SEQ ID NO: 412 comprises a HCDR1 of SEQ ID NO: 308; a HCDR2 of SEQ ID NO: 310; a HCDR3 of SEQ ID NO: 312; a LCDR1 of SEQ ID NO: 316; a LCDR2 of KAS and a LCDR3 of SEQ ID NO: 320.

In one embodiment, the at least one human antibody or antigen-binding fragment thereof that binds to chain 2 of Fel d1 interacts with amino acid residue numbers ranging from about residue 12 through residue 54 of SEQ ID NO: 396.

In one embodiment, the at least one human antibody or antigen-binding fragment thereof that binds to chain 2 of Fel d1 interacts with amino acid residue numbers ranging from about residue 21 through residue 47 of SEQ ID NO: 396.

In one embodiment, the at least one human antibody or antigen-binding fragment thereof that binds to chain 2 of Fel d1 interacts with at least one or more of the following amino acid residues in SEQ ID NO: 396: the N at position 21, the E at position 22, the L at position 23, the L at position 24, the D at position 26, the L at position 27, the T at position 30, the K at position 31, the E at position 36, the R at position 39, the K at position 43, the D at position 47.

In one embodiment, the at least one human antibody or antigen-binding fragment thereof that binds to chain 2 of Fel d1 binds to an epitope that comprises a plurality of the following amino acid residues from SEQ ID NO: 396: N21, E22, L23, L24, D26, L27, T30, K31, E36, R39, K43, D47.

In one embodiment, the at least one human antibody or antigen-binding fragment thereof that interacts with at least one or more of the following amino acid residues of SEQ ID NO: 396, including the N at position 21, the E at position 22, the L at position 23, the L at position 24, the D at position 26, the L at position 27, the T at position 30, the K at position 31, the E at position 36, the R at position 39, the K at position 43, the D at position 47, comprises the HCVR/LCVR amino acid sequence pair of SEQ ID NOs: 306/314.

In certain embodiments, the epitopes to which the anti-Fel d1 antibodies bind are identified using X-ray crystallographic analysis.

In one embodiment, the at least one human antibody or antigen binding fragment thereof that binds specifically to Fel d1 comprises the HCDR1, HCDR2 and HCDR3 amino acid sequences of SEQ ID NO: 20, 22 and 24, respectively and LCDR1, LCDR2 and LCDR3 amino acid sequences of SEQ ID NO: 28, AAS, and SEQ ID NO: 32, respectively.

In one embodiment, the at least one human antibody or antigen binding fragment thereof that binds specifically to Fel d1 comprises the HCDR1, HCDR2 and HCDR3 amino acid sequences of SEQ ID NO: 308, 310 and 312, respectively and LCDR1, LCDR2 and LCDR3 amino acid sequences of SEQ ID NO: 316, KAS, and SEQ ID NO: 320, respectively.

In one embodiment, a method according to the invention employs at least one fully human monoclonal antibody or antigen-binding fragment thereof that binds specifically to Fel d1, wherein the antibody or fragment thereof exhibits one or more of the following characteristics: (i) comprises a HCVR having an amino acid sequence selected from the group consisting of SEQ ID NO: 18, 66, 130, 162, 242, 306, 322, 370 and 460, or a substantially similar sequence thereof having at least 90%, at least 95%, at least 98% or at least 99% sequence identity; (ii) comprises a LCVR having an amino acid sequence selected from the group consisting of SEQ ID NO: 26, 74, 138, 170, 250, 314, 330, 378 and 468, or a substantially similar sequence thereof having at least 90%, at least 95%, at least 98% or at least 99% sequence identity; (iii) comprises a HCDR3 domain having an amino acid sequence selected from the group consisting of SEQ ID NO: 24, 72, 136, 168, 248, 312, 328, 376 and 466, or a substantially similar sequence thereof having at least 90%, at least 95%, at least 98% or at least 99% sequence identity; and a LCDR3 domain having an amino acid sequence selected from the group consisting of SEQ ID NO: 32, 80, 144, 176, 256, 320, 336, 384 and 474, or a substantially similar sequence thereof having at least 90%, at least 95%, at least 98% or at least 99% sequence identity; (iv) comprises a HCDR1 domain having an amino acid sequence selected from the group consisting of SEQ ID NO: 20, 68, 132, 164, 244, 308, 324, 372 and 462, or a substantially similar sequence thereof having at least 90%, at least 95%, at least 98% or at least 99% sequence identity; a HCDR2 domain having an amino acid sequence selected from the group consisting of SEQ ID NO: 22, 70, 134, 166, 246, 310, 326, 374 and 464, or a substantially similar sequence thereof having at least 90%, at least 95%, at least 98% or at least 99% sequence identity; a LCDR1 domain having an amino acid sequence selected from the group consisting of SEQ ID NO: 28, 76, 140, 172, 252, 316, 332, 380 and 470, or a substantially similar sequence thereof having at least 90%, at least 95%, at least 98% or at least 99% sequence identity; and a LCDR2 domain having an amino acid sequence selected from the group consisting of AAS, VVTS, DAS, AAS, YAS, KAS, GAS, SAS, and KAS, or a substantially similar sequence thereof having at least 90%, at least 95%, at least 98% or at least 99% sequence identity; (v) binds to Fel d1 with a K_{D} equal to or less than 10⁻⁶ M and preferably equal to or less than 10⁻⁹ M; (vi) demonstrates efficacy in at least one animal model of anaphylaxis or inflammation; or (vii) competes with a reference antibody for binding to Fel d1.

In one embodiment, a “reference antibody” may include, for example, antibodies having a combination of heavy chain and light chain amino acid sequence pairs selected from the group consisting of 18/26, 66/74, 130/138, 162/170, 242/250, 306/314, 322/330, 370/378 and 460/468.

In one embodiment, a method according to the invention employs at least one human antibody or antigen-binding fragment specific for Fel d1, comprising a HCVR encoded by nucleotide sequence segments derived from VH, DH and JH germline sequences, and a LCVR encoded by nucleotide sequence segments derived from VK and JK germline sequences.

The invention encompasses antibodies having a modified glycosylation pattern. In some applications, modification to remove undesirable glycosylation sites may be useful, or e.g., removal of a fucose moiety to increase antibody dependent cellular cytotoxicity (ADCC) function (see Shield, et al. (2002) JBC 277:26733). In other applications, modification of galactosylation can be made in order to modify complement dependent cytotoxicity (CDC).

In another embodiment, methods according to the invention employ at least one antibody or antigen-binding fragment thereof that specifically binds to Fel d1, wherein the isolated antibody or antigen-binding fragment thereof competes for specific binding to Fel d1 with an antibody or antigen-binding fragment comprising the complementarity determining regions (CDRs) of a heavy chain variable region (HCVR), wherein the HCVR has an amino acid sequence selected from the group consisting of SEQ ID NOs: 2, 18, 34, 50, 66, 82, 98, 114, 130, 146, 162, 178, 194, 210, 226, 242, 258, 274, 290, 306, 322, 338, 354, 370 and 460; and the CDRs of a light chain variable region (LCVR), wherein the LCVR has an amino acid sequence selected from the group consisting of SEQ ID NOs: 10, 26, 42, 58, 74, 90, 106, 122, 138, 154, 170, 186, 202, 218, 234, 250, 266, 282, 298, 314, 330, 346, 362, 378 and 468.

In another embodiment, methods according to the invention employ at least one isolated antibody or antigen-binding fragment thereof that competes for specific binding to Fel d1 with an antibody or antigen-binding fragment comprising the complementarity determining regions (CDRs) of a heavy chain variable region (HCVR), wherein the HCVR has an amino acid sequence selected from the group consisting of SEQ ID NOs: 18, 66, 130, 162, 242, 306, 322, 370 and 460; and the CDRs of a light chain variable region (LCVR), wherein the LCVR has an amino acid sequence selected from the group consisting of SEQ ID NOs: 26, 74, 138, 170, 250, 314, 330, 378 and 468.

In another embodiment, methods according to the invention employ at least one isolated antibody or antigen-binding fragment thereof that competes for specific binding to Fel d1 with an antibody or antigen-binding fragment comprising the heavy and light chain CDRs contained within heavy and light chain sequence pairs selected from the group consisting of SEQ ID NOs: 18/26, 66/74, 130/138, 162/170, 242/250, 306/314, 322/330, 370/378 and 460/468.

In another embodiment, methods according to the invention employ at least one isolated antibody or antigen-binding fragment thereof that binds the same epitope on Fel d1 as an antibody or antigen-binding fragment comprising the complementarity determining regions (CDRs) of a heavy chain variable region (HCVR), wherein the HCVR has an amino acid sequence selected from the group consisting of SEQ ID NOs: 2, 18, 34, 50, 66, 82, 98, 114, 130, 146, 162, 178, 194, 210, 226, 242, 258, 274, 290, 306, 322, 338, 354, 370 and 460; and the CDRs of a light chain variable region (LCVR), wherein the LCVR has an amino acid sequence selected from the group consisting of SEQ ID NOs: 10, 26, 42, 58, 74, 90, 106, 122, 138, 154, 170, 186, 202, 218, 234, 250, 266, 282, 298, 314, 330, 346, 362, 378 and 468.

In another embodiment, methods according to the invention employ at least one isolated antibody or antigen-binding fragment thereof that binds the same epitope on Fel d1 as an antibody or antigen-binding fragment comprising the complementarity determining regions (CDRs) of a heavy chain variable region (HCVR), wherein the HCVR has an amino acid sequence selected from the group consisting of SEQ ID NOs: 18, 66, 130, 162, 242, 306, 322, 370 and 460; and the CDRs of a light chain variable region (LCVR), wherein the LCVR has an amino acid sequence selected from the group consisting of SEQ ID NOs: 10, 26, 42, 58, 74, 90, 106, 122, 138, 154, 170, 186, 202, 218, 234, 250, 266, 282, 298, 314, 330, 346, 362, 378 and 468.

In another embodiment, methods according to the invention employ at least one isolated antibody or antigen-binding fragment thereof that binds the same epitope on Fel d1 as an antibody or antigen-binding fragment comprising the heavy and light chain CDRs contained within heavy and light chain sequence pairs selected from the group consisting of SEQ ID NOs: 18/26, 66/74, 130/138, 162/170, 242/250, 306/314, 322/330, 370/378 and 460/468.

In certain embodiments of methods according to the invention, “at least one” in the context of the antibody or antigen-binding fragment thereof refers to more than one antibody or antigen-binding fragments, for example, two, that specifically bind the allergen. In certain embodiments, when more than one antibody or antigen binding fragment thereof is utilized, the antibodies do not compete with one another for binding to the allergen. In other embodiments, the antibodies or antigen binding fragments thereof specifically bind to different epitopes. In certain embodiments, one or more antibodies or antigen binding fragments thereof specifically bind to different epitopes on Fel d1. In embodiments, a pharmaceutical composition comprises one antibody or antigen binding fragment thereof that binds to an epitope comprising amino acids 15 to 54 of SEQ ID NO:396, and a second antibody or antigen binding fragment thereof that binds to an epitope comprising amino acids 113 to 116 of SEQ ID NO:396.

In another aspect, the invention provides a pharmaceutical composition comprising a therapeutically effective amount of one or more isolated human antibodies or antigen-binding fragments thereof that specifically bind Fel d1, together with one or more pharmaceutically acceptable excipients. In another embodiment, the composition is for use in a method according to the invention.

In one embodiment, the pharmaceutical composition comprises a therapeutically effective amount of two isolated human antibodies or antigen-binding fragments thereof that specifically bind Fel d1 together with one or more pharmaceutically acceptable excipients.

In one embodiment, the pharmaceutical composition comprises:
(a) an isolated first fully human monoclonal antibody, or antigen-binding fragment thereof that specifically binds Fel d1, which comprises a HCVR having an amino acid sequence as set forth is SEQ ID NO: 18; and a LCVR having an amino acid sequence as set forth is SEQ ID NO: 26; and
(b) an isolated second fully human monoclonal antibody, or antigen-binding fragment thereof that specifically binds Fel d1, which comprises a HCVR having an amino acid sequence as set forth in SEQ ID NO: 306; and a LCVR having an amino acid sequence as set forth in SEQ ID NO: 314.

In one embodiment, the pharmaceutical composition further comprises a therapeutically effective amount of a second therapeutic agent. The second therapeutic agent may be a small molecule drug, a protein/polypeptide, an antibody, a nucleic acid molecule, such as an anti-sense molecule, or a siRNA. The second therapeutic agent may be synthetic or naturally derived.

The second therapeutic agent may be any agent that is advantageously combined with an antibody or fragment thereof of the invention, for example, a second antibody other than those described herein that is capable of blocking the binding of Fel d1 to IgE present on mast cells or basophils. A second therapeutic agent may also be any agent that is used as standard of care in treating an allergic response to any allergen. Such second therapeutic agent may be an antihistamine, epinephrine, a decongestant, a corticosteroid, or a peptide vaccine.

In certain embodiments, the second therapeutic agent may be an agent that helps to counteract or reduce any possible side effect(s) associated with the antibody or antigen-binding fragment of an antibody of the invention, if such side effect(s) should occur.

When multiple therapeutics are co-administered, dosages may be adjusted accordingly, as is recognized in the pertinent art.

In another aspect, the invention provides for use of a pharmaceutical composition as described herein in the manufacture of a medicament.

Other embodiments will become apparent from a review of the ensuing detailed description.

### BRIEF DESCRIPTION OF THE FIGURES

FIGS. 1A, 1B, and 1C shows study flow diagrams for Example 5. R=randomization; d=day; wk=week. (FIG. 1A) provides an overview of the study design. (FIG. 1B) provides a flow diagram for the EEU exposure visit. (FIG. 1C) provides a diagram of the allergen exposure during the EEU exposure visit. Cat-allergic patients with mild asthma (N=56; 18-65 years) underwent a baseline EEU exposure with cat allergen for up to 2 hours or until they experienced an Early Asthmatic Response (EAR), defined as a reduction in FEV1 ≥20% from baseline, whereupon they were treated with rescue medications. Those who experienced an EAR within 2 hours at the baseline EEU exposure with cat allergen were randomized to receive a single dose of REGN1908-1909 (n=29) or placebo (n=27) prior to cat-allergen exposures in an EEU for up to 4 hours at 1 week, 4 weeks, 8 weeks, and 12 weeks.

FIG. 2 shows a schedule of events for Example 5. ACT=asthma control test; EEU=environmental exposure unit; PBMC=peripheral blood mononuclear cell; R=randomization; V=visit.

FIG. 3 shows a summary of patient/subject demographics for Example 5.

FIG. 4 shows a summary of patient/subject baseline characteristics for Example 5. Patients' baseline characteristics were generally well balanced between the REGN1908-1909 and placebo treatment groups. FEV1=forced expiratory volume in 1 second; TNSS=total nasal symptom score; TOSS=total ocular symptom score; AUC=area under the curve.

FIGS. 5A and 5B show change in chest tightness (FIG. 5A) and breathing difficulty (FIG. 5B) during cat allergen exposure in an EEU. ^{a}P-values vs. placebo. AUC, area under the curve; EEU, environmental exposure unit; LS, least squares; SE, standard error.

FIGS. 6A and 6B shows that single dose REGN1908-1909 prevented cat allergen-induced declines in FEV1. (FIG. 6A) percent change from pre-challenge baseline FEV1 during all EEU challenges in REGN1908-1909-treated patients (LOCF (last observation carried forward) analysis). (FIG. 6B) percent change from pre-challenge baseline FEV1 during all EEU challenges in placebo-treated patients (LOCF (last observation carried forward) analysis).

FIG. 7 shows mean cat allergen exposure tolerated by subjects during EEU challenges comparing treatment with REGN1908-1909 versus placebo. Single dose REGN1908-1909 increased cumulative cat allergen quantity tolerated at 1 week and for up to 3 months. Cat allergen exposure tolerated=minute ventilation (L/min)*×allergen concentration (ng/m³)×time in EEU (minutes). *Where 1 L/min=0.001 m³/min.

FIG. 8 shows the mean percent change in the AUC of SPT with serial cat allergen titration from baseline to days 29, 85, and 113. AUC of the SPT was measured by the mean wheal diameter (mm), adjusted by the negative control mean wheal diameter, calculated over the log-transformed cat allergen concentrations (from highest to lowest). AUC, area under the curve; LS, least squares; SE, standard error; SPT, skin prick test.

FIG. 9 shows the proportion of patients receiving rescue medication at EEU exit.

### DETAILED DESCRIPTION

Before the present methods are described, it is to be understood that this invention is not limited to particular methods, and experimental conditions described, as such methods and conditions may vary. It is also to be understood that the terminology used herein is for the purpose of describing particular embodiments only, and is not intended to be limiting, since the scope of the present invention will be limited only by the appended claims. Unless defined otherwise, all technical and scientific terms used herein have the same meaning as commonly understood by one of ordinary skill in the art to which this invention belongs.

As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

Although any methods and materials similar or equivalent to those described herein can be used in the practice or testing of the present invention, preferred methods and materials are now described. All publications mentioned herein are incorporated herein by reference in their entirety.

### Definitions

The term “Fel d1” or “FELD1” or “Fel d1” or “Fel d1” or “fel d1”, as used herein, refers to at least one Fel d1 protein, either in natural/native form, or recombinantly produced. The Fel d1 protein comprises, or alternatively consists of, chain 1 (also referred to as chain A) of Fel d1 (SEQ ID NO: 392) and chain 2 (also referred to as chain B) of Fel d1 (SEQ ID NO: 393). The natural Fel d1 protein is an approximately 18 kDa heterodimeric glycoprotein composed of two chains derived from two independent genes (See Duffort, O. A., et al., (1991), Mol. Immunol. 28:301-309; Kristensen, A. K., et al., (1997), Biol. Chem. 378:899-908; Kaiser L., et al., (2003), J. Biol. Chem. 278(39):37730-37735). A recombinantly produced Fel d1 protein is also shown as SEQ ID NO: 396, wherein this sequence contains amino acid residues 18 through 109 of Fel d1 chain B from GenBank accession number NP 001041619.1 (without the signal sequence) fused in line with amino acid residues 19-88 of chain A of Fel d1 from GenBank accession number NP 001041618.1 (without the signal sequence and with a D27G mutation, which corresponds to the glycine at position 101 of SEQ ID NO: 396). Other recombinantly produced Fel d1 constructs of the invention are exemplified in SEQ ID NOs: 385, 394, 395 and 397

“Chain 1”, or “chain A” of Fel d1 is a polypeptide comprising, or alternatively consisting of, an amino acid sequence of SEQ ID NO: 392, or a homologous sequence thereof. The term homologous sequence of SEQ ID NO:392, as used herein, refers to a polypeptide that has an identity to SEQ ID NO:392 which is greater than 70%, preferably greater than 80%, more preferably greater than 90%, and even more preferably greater than 95%. The amino acid sequence of chain 1 of Fel d1 is also provided in GenBank as accession number P30438, or as accession number NP_001041618.1, which also include the signal peptide that is removed in the mature protein.

“Chain 2”, or “chain B” of Fel d1 is a polypeptide comprising, or alternatively consisting of, an amino acid sequence of SEQ ID NO: 393, or a homologous sequence thereof. The term homologous sequence of SEQ ID NO: 393, as used herein, refers to a polypeptide that has an identity to SEQ ID NO:393 which is greater than 70%, preferably greater than 80%, more preferably greater than 90%, and even more preferably greater than 95%. The amino acid sequence of chain 2 of Fel d1 is also provided in GenBank as accession number P30440, or as accession number NP_001041619.1, which include the signal peptide that is removed in the mature protein.

The term “Fel d1 fragment” as used herein, refers to a polypeptide comprising or alternatively consisting of, at least one antigenic site of Fel d1. In one embodiment, the term “Fel d1 fragment” as used herein, refers to a polypeptide comprising or alternatively consisting of at least two antigenic sites of Fel d1. In one embodiment, the antigenic sites are covalently linked. In one embodiment, the antigenic sites are linked by at least one peptide bond. In one embodiment, the two antigenic sites are linked by at least one peptide bond and a spacer between the antigenic sites. In one embodiment, the at least two antigenic sites derive from both chain 1 of Fel d1 and from chain 2 of Fel d1. In one embodiment, the at least two antigenic sites comprise amino acid sequences 23-92 of GenBank accession number P30438 and amino acid sequences 18-109 of GenBank accession number P30440. In one embodiment, the at least two antigenic sites derive from both chain 1 of Fel d1 and from chain 2 of Fel d1. In one embodiment, the at least two antigenic sites comprise amino acid sequences 19-88 of GenBank accession number NP_001041618.1 and amino acid sequences 18-109 of GenBank accession number NP_001041619.1. In one embodiment, the at least two antigenic sites comprise an amino acid sequence within any of SEQ ID NOs: 385, 394, 395, 396 or 397. In one embodiment, any of the Fel d1 fragments are capable of inducing the production of antibodies in vivo that specifically bind to naturally occurring Fel d1, or to recombinantly produced Fel d1.

The term “antibody”, as used herein, means any antigen-binding molecule or molecular complex comprising at least one complementarity determining region (CDR) that specifically binds to or interacts with a particular antigen (e.g., Fel d1). The term “antibody”, as used herein, is intended to refer to immunoglobulin molecules comprised of four polypeptide chains, two heavy (H) chains and two light (L) chains inter-connected by disulfide bonds (i.e., “full antibody molecules”), as well as multimers thereof (e.g., IgM) or antigen-binding fragments thereof. Each heavy chain is comprised of a heavy chain variable region (“HCVR” or “V_{H}”) and a heavy chain constant region (comprised of domains C_{H}1, C_{H}2 and C_{H}3). Each light chain is comprised of a light chain variable region (“LCVR or “V_{L}”) and a light chain constant region (C_{L}). The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In certain embodiments of the invention, the FRs of the antibody (or antigen binding fragment thereof) may be identical to the human germline sequences, or may be naturally or artificially modified. An amino acid consensus sequence may be defined based on a side-by-side analysis of two or more CDRs.

Substitution of one or more CDR residues or omission of one or more CDRs is also possible. Antibodies have been described in the scientific literature in which one or two CDRs can be dispensed with for binding. Padlan, et al., (1995 FASEB J. 9:133-139) analyzed the contact regions between antibodies and their antigens, based on published crystal structures, and concluded that only about one fifth to one third of CDR residues actually contact the antigen. Padlan also found many antibodies in which one or two CDRs had no amino acids in contact with an antigen (see also, Vajdos, et al., (2002), J Mol Biol 320:415-428).

CDR residues not contacting antigen can be identified based on previous studies (for example residues H60-H65 in CDRH2 are often not required), from regions of Kabat CDRs lying outside Chothia CDRs, by molecular modeling and/or empirically. If a CDR or residue(s) thereof is omitted, it is usually substituted with an amino acid occupying the corresponding position in another human antibody sequence or a consensus of such sequences. Positions for substitution within CDRs and amino acids to substitute can also be selected empirically. Empirical substitutions can be conservative or non-conservative substitutions.

The fully human monoclonal antibodies that specifically bind to Fel d1, as described herein, may comprise one or more amino acid substitutions, insertions and/or deletions in the framework and/or CDR regions of the heavy and light chain variable domains as compared to the corresponding germline sequences. Such mutations can be readily ascertained by comparing the amino acid sequences disclosed herein to germline sequences available from, for example, public antibody sequence databases. The present invention employs antibodies, and antigen-binding fragments thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments that comprise one or more individual germline mutations or combinations thereof. In certain embodiments, all of the framework and/or CDR residues within the V_{H} and/or V_{L} domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies used in the methods of the present invention may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, etc. Antibodies and antigen-binding fragments obtained in this general manner are encompassed within the present invention.

The present invention also employs fully human monoclonal antibodies comprising variants of any of the HCVR, LCVR, and/or CDR amino acid sequences disclosed herein having one or more conservative substitutions. For example, the present invention uses antibodies having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences disclosed herein.

The term “human antibody”, as used herein, is intended to include antibodies having variable and constant regions derived from human germline immunoglobulin sequences. The human mAbs of the invention may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include mAbs in which CDR sequences derived from the germline of another mammalian species (e.g., mouse), have been grafted onto human FR sequences.

As used herein, the expression “antigen-binding molecule” means a protein, polypeptide or molecular complex comprising or consisting of at least one complementarity determining region (CDR) that alone, or in combination with one or more additional CDRs and/or framework regions (FRs), specifically binds to a particular antigen. In certain embodiments, an antigen-binding molecule is an antibody or a fragment of an antibody, as those terms are defined elsewhere herein.

As used herein, the expression “bi-specific antigen-binding molecule” means a protein, polypeptide or molecular complex comprising at least a first antigen-binding domain and a second antigen-binding domain (i.e., two arms). Each antigen-binding domain within the bi-specific antigen-binding molecule comprises at least one CDR that alone, or in combination with one or more additional CDRs and/or FRs, specifically binds to a particular antigen. In the context of the present invention, the first antigen-binding domain specifically binds a first antigen on Fel d1 and the second antigen-binding domain specifically binds a second, distinct antigen on Fel d1.

The term “specifically binds,” or “binds specifically to”, or the like, means that an antibody or antigen-binding fragment thereof forms a complex with an antigen that is relatively stable under physiologic conditions. Specific binding can be characterized by an equilibrium dissociation constant of at least about 1×10⁻⁶ M or less (e.g., a smaller K D denotes a tighter binding). Methods for determining whether two molecules specifically bind are well known in the art and include, for example, equilibrium dialysis, surface plasmon resonance, and the like. As described herein, antibodies have been identified by surface plasmon resonance, e.g., BIACORE™, which bind specifically to Fel d1. Moreover, multi-specific antibodies that bind to Fel d1 and one or more additional antigens or a bi-specific that binds to two different regions of Fel d1 (for example, chain 1 and/or chain 2 of Fel d1) are nonetheless considered antibodies that “specifically bind”, as used herein.

The term “high affinity” antibody refers to those mAbs having a binding affinity to Fel d1, expressed as K_{D}, of at least 10⁻⁸ M; preferably 10⁻⁹ M; more preferably 10⁻¹⁹ M, even more preferably 10⁻¹¹ M, even more preferably 10⁻¹² M, as measured by surface plasmon resonance, e.g., BIACORE™ or solution-affinity ELISA.

By the term “slow off rate”, “Koff” or “kd” is meant an antibody that dissociates from Fel d1, with a rate constant of 1×10⁻³ s⁻¹ or less, preferably 1×10⁻⁴ s⁻¹ or less, as determined by surface plasmon resonance, e.g., BIACORE™.

The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. The terms “antigen-binding portion” of an antibody, or “antibody fragment”, as used herein, refers to one or more fragments of an antibody that retain the ability to bind to the allergen, e.g., Fel d1.

The specific embodiments, antibody or antibody fragments of the invention may be conjugated to a therapeutic moiety (“immunoconjugate”), such as a corticosteroid, a second anti-allergen antibody (e.g., Fel d1), or epinephrine, a vaccine, or any other therapeutic moiety useful for treating an allergic response to an allergen, e.g., Fel d1.

An “isolated antibody”, as used herein, is intended to refer to an antibody that is substantially free of other antibodies (Abs) having different antigenic specificities (e.g., an isolated antibody that specifically binds, e.g., Fel d1, or a fragment thereof, is substantially free of Abs that specifically bind antigens other than Fel d1).

A “blocking antibody” or a “neutralizing antibody”, as used herein (or an “antibody that neutralizes Fel d1 activity”), is intended to refer to an antibody, or an antigen binding portion thereof, whose binding to Fel d1 results in inhibition of at least one biological activity of Fel d1. For example, an antibody of the invention may aid in preventing the primary allergic response to Fel d1. Alternatively, an antibody of the invention may demonstrate the ability to prevent a secondary allergic response to Fel d1, or at least one symptom of an allergic response to Fel d1, including sneezing, coughing, an asthmatic condition, or an anaphylactic response caused by Fel d1. This inhibition of the biological activity of Fel d1 can be assessed by measuring one or more indicators of Fel d1 biological activity by one or more of several standard in vitro or in vivo assays (such as a passive cutaneous anaphylaxis assay, as described herein) or other in vivo assays known in the art (for example, other animal models to look at protection from challenge with Fel d1 following administration of one or more of the antibodies described herein).

The term “surface plasmon resonance”, as used herein, refers to an optical phenomenon that allows for the analysis of real-time biomolecular interactions by detection of alterations in protein concentrations within a biosensor matrix, for example using the BIACORE™ system (Pharmacia Biosensor AB, Uppsala, Sweden and Piscataway, N.J.).

The term “K_{D}”, as used herein, is intended to refer to the equilibrium dissociation constant of a particular antibody-antigen interaction.

The term “epitope” refers to an antigenic determinant that interacts with a specific antigen-binding site in the variable region of an antibody molecule known as a paratope. A single antigen may have more than one epitope. Thus, different antibodies may bind to different areas on an antigen and may have different biological effects. The term “epitope” also refers to a site on an antigen to which B and/or T cells respond. It also refers to a region of an antigen that is bound by an antibody. Epitopes may be either linear or conformational. A linear epitope is one produced by adjacent amino acid residues in a polypeptide chain. A conformational epitope is produced by spatially juxtaposed amino acids from different segments of the linear polypeptide chain. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics. Epitopes may also be defined as structural or functional. Functional epitopes are generally a subset of the structural epitopes and have those residues that directly contribute to the affinity of the interaction. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5 or 8-10 amino acids in a unique spatial conformation.

The term “substantial identity” or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 90%, and more preferably at least about 95%, 96%, 97%, 98% or 99% of the nucleotide bases, as measured by any well-known algorithm of sequence identity, such as FASTA, BLAST or GAP, as discussed below. A nucleic acid molecule having substantial identity to a reference nucleic acid molecule may, in certain instances, encode a polypeptide having the same or substantially similar amino acid sequence as the polypeptide encoded by the reference nucleic acid molecule.

As applied to polypeptides, the term “substantial similarity” or “substantially similar” means that two peptide sequences, when optimally aligned, such as by the programs GAP or BESTFIT using default gap weights, share at least 90% sequence identity, even more preferably at least 95%, 98% or 99% sequence identity. Preferably, residue positions, which are not identical, differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent or degree of similarity may be adjusted upwards to correct for the conservative nature of the substitution. Means for making this adjustment are well known to those of skill in the art. See, e.g., Pearson (1994) Methods Mol. Biol. 24: 307-331, which is herein incorporated by reference. Examples of groups of amino acids that have side chains with similar chemical properties include 1) aliphatic side chains: glycine, alanine, valine, leucine and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartate and glutamate, and 7) sulfur-containing side chains: cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet, et al., (1992) Science 256: 1443 45, herein incorporated by reference. A “moderately conservative” replacement is any change having a nonnegative value in the PAM250 log-likelihood matrix.

Sequence similarity for polypeptides is typically measured using sequence analysis software. Protein analysis software matches similar sequences using measures of similarity assigned to various substitutions, deletions and other modifications, including conservative amino acid substitutions. For instance, GCG software contains programs such as GAP and BESTFIT, which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA with default or recommended parameters; a program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence of the invention to a database containing a large number of sequences from different organisms is the computer program BLAST, especially BLASTP or TBLASTN, using default parameters. See, e.g., Altschul, et al. (1990) J. Mol. Biol. 215: 403 410 and (1997) Nucleic Acids Res. 25:3389 402, each of which is herein incorporated by reference.

In specific embodiments, the antibody or antibody fragment for use in a method of the invention may be mono-specific, bi-specific, or multi-specific. Multi-specific antibodies may be specific for different epitopes of one target polypeptide or may contain antigen-binding domains specific for epitopes of more than one target polypeptide. An exemplary bi-specific antibody format that can be used in the context of the present invention involves the use of a first immunoglobulin (Ig) C_{H}3 domain and a second Ig C_{H}3 domain, wherein the first and second Ig C_{H}3 domains differ from one another by at least one amino acid, and wherein at least one amino acid difference reduces binding of the bi-specific antibody to Protein A as compared to a bi-specific antibody lacking the amino acid difference. In one embodiment, the first Ig C_{H}3 domain binds Protein A and the second Ig C_{H}3 domain contains a mutation that reduces or abolishes Protein A binding such as an H95R modification (by IMGT exon numbering; H435R by EU numbering). The second C_{H}3 may further comprise an Y96F modification (by IMGT; Y436F by EU). Further modifications that may be found within the second C_{H}3 include: D16E, L18M, N44S, K52N, V57M, and V821 (by IMGT; D356E, L358M, N384S, K392N, V397M, and V4221 by EU) in the case of IgG1 mAbs; N44S, K52N, and V821 (IMGT; N384S, K392N, and V4221 by EU) in the case of IgG2 mAbs; and Q15R, N44S, K52N, V57M, R69K, E79Q, and V821 (by IMGT; Q355R, N384S, K392N, V397M, R409K, E419Q, and V4221 by EU) in the case of IgG4 mAbs. Variations on the bi-specific antibody format described above are contemplated within the scope of the present invention.

By the phrase “therapeutically effective amount” is meant an amount that produces the desired effect for which it is administered. The exact amount will depend on the purpose of the treatment, and it will be ascertainable by one skilled in the art using known techniques (see, for example, Lloyd (1999) The Art, Science and Technology of Pharmaceutical Compounding).

The antibodies of the invention may be used to “desensitize” a cat-sensitive individual. The term to “desensitize” is defined herein as to decrease the allergic-reactivity of a cat-sensitive individual to exposure to cats, cat dander or products thereof, e.g., Fel d1 (to a level less than that which the cat-sensitive individual would otherwise experience).

The “Total Nasal Symptom Score” (TNSS); with a possible score of 0-12, is based on assessment of 4 symptoms graded on a 0 (none) to 3 (severe) scale for congestion, itching and rhinorrhea, and 0 (none) to 3 (5 or more sneezes) for sneezing. Each of the 4 symptoms is evaluated using the following scale of 0=None, 1=Mild, 2=Moderate, or 3=Severe.

A “Visual Analog Scale” (VAS) is a quantitative measure largely validated in many diseases. These scales have been extensively used to assess the severity of rhinitis as well as the efficacy of therapeutic intervention. A VAS ranging from 0 (no nasal symptoms) to 100 cm (maximal nasal symptoms) was used to assess the severity of combined nasal symptoms.

The “Peak Nasal Inspiratory Flow” (PNIF) is an objective method, which uses a nasal spirometer for measuring nasal airflow (measured as I/min).

The “Titrated Skin Prick Test” (SPT) is a reliable method to diagnose IgE-mediated allergic disease in patients with rhinoconjunctivitis, asthma, urticaria, anaphylaxis, atopic eczema and suspected food and drug allergy. It provides evidence for sensitization and can help to confirm the diagnosis of a suspected type I allergy. SPT interpretation utilizes the presence and degree of cutaneous reactivity as a surrogate marker for sensitization within target organs, i.e., eyes, nose, lung, gut and skin. When relevant allergens are introduced into the skin, specific IgE bound to the surface receptors on mast cells are cross-linked, mast cells degranulate, and histamine and other mediators are released. This produces a wheal and flare response, which can be quantitated. SPT results correlate with those of nasal challenge which may also be used as a surrogate to test clinically relevant sensitization. (Bousquet J, et al., (1987), Clin Allergy, 17(6):529-536).

### General Description

Disclosed herein is the evaluation of the prophylactic efficacy of REGN1908-1909 (anti-Fel d1) administered as a single dose on day 1 in cat-allergic asthmatic patients not living with a cat in the prevention of a Controlled Cat Allergen Challenge-induced early asthmatic response (EAR) assessed by measures of lung function (FEV1) compared to placebo-treated patients on day 8. REGN1908-1909 is, in specific embodiments, designed to specifically bind and block the Fel d1 allergen, thus preventing it from binding and being triggered by the endogenous antibodies that cause allergies (i.e., IgE antibodies)

Additionally disclosed herein is the evaluation of the prophylactic efficacy of REGN1908-1909 administered as a single dose on day 1 in cat-allergic asthmatic patients not living with a cat, in the prevention of a Controlled Cat Allergen Challenge-induced:
Early asthmatic response (EAR) assessed by measures of lung function (FEV1) compared to placebo-treated patients on days 29, 57, and 85;
Allergic rhinitis symptoms assessed by total nasal symptom score (TNSS) compared to placebo patients on days 8, 29, 57, and 85;
Ocular symptoms assessed by total ocular symptom score (TOSS) compared to placebo patients on days 8, 29, 57, and 85.

Further disclosed herein is the evaluation of the prophylactic efficacy of REGN1908-1909 administered as a single dose on day 1 in cat-allergic asthmatic patients not living with a cat to increase the exposure to cat allergen, measured as a product of minute ventilation and time, required to induce EAR in a Controlled Cat Allergen Challenge (40 ng/m³ Fel d1 allergen×minute ventilation×time) as compared to placebo patients on days 8, 29, 57, and 85.

Still further disclosed herein is the evaluation of the safety and tolerability of REGN1908-1909 vs. placebo in patients with cat allergen-triggered asthma.

The study described herein aims to investigate the prophylactic effect of REGN1908-1909 to reduce bronchoconstriction in cat-allergic patients with mild asthma when exposed to cat allergen in a controlled environmental exposure unit (EEU). Environmental exposure units are enclosed spaces that control temperature, air flow, and humidity, and provide diffuse allergen exposure to simulate natural circumstances. Because EEUs provide allergen exposures that are standardized and reproducible, they have been developed for the investigation of mechanisms and treatment of allergies (Pfaar, et al., 2017 Allergy 72(7): 1035-1042). Globally to date, one EEU clinical trial site has been developed and validated to study asthmatic responses to cat allergen exposures (Alyatech, Strasbourg, France). The EEU is optimally designed to study patients with asthma upon allergen exposure with real-time electronic monitoring of FEV1 using hand-held spirometers (Medical International Research Spirobank II) and continuous, direct visualization of patients by clinic staff. Additionally, medications to treat asthma, allergy, and anaphylaxis are readily available.

### Fel d1 Cat Allergen

The domestic cat is a source of many indoor allergens and the severity of the symptoms in individuals who demonstrate a sensitivity to cat allergens ranges from a relatively mild rhinitis and conjunctivitis to a potentially life-threatening asthmatic condition (Lau, S., et al., (2000), Lancet 356:1392-1397). While patients who demonstrate such a sensitivity to cats appear to be responsive to different molecules found in cat dander and pelts, the major allergen appears to be Fel d1 (Felis domesticus allergen 1). It has been shown that greater than 80% of patients who are allergic to cats have IgE antibodies to this allergen (van Ree, R., et al., (1999), J. Allergy Clin. Immunol 104:1223-1230).

The Fel d1 protein is an approximately 18 kDa heterodimeric acidic glycoprotein that contains about 10-20% of N-linked carbohydrates. Each heterodimer comprises two polypeptide chains that are encoded by two separate genes (Duffort, O A, et al., (1991), Mol. Immunol. 28:301-309; Morgenstern, J P, et al., (1991), PNAS 88:9690-9694; Griffith, I. J., et al., (1992), Gene 113:263-268). Chain 1 comprises about 70 amino acid residues and chain 2 comprises about 90-92 amino acid residues. Three interchain disulfide bonds linking the two chains in natural Fel d1 have been proposed (Kristensen, A. K., et al., (1997), Biol. Chem. 378:899-908) and confirmed for recombinant Fel d1 in the crystal structure (Kaiser, L., et al., (2003), J. Biol. Chem. 278:37730-37735; Kaiser, L., et al., (2007), J. Mol. Biol. 370:714-727). Although each chain is sometimes individually referred to as “Fel d1”, both chains are needed for the full protein allergen.

Fel d1 is produced by sebaceous glands, squamous glands and squamous epithelial cells and is transferred to the pelt by licking and grooming (Bartholome, K., et al., (1985), J. Allergy Clin. Immunol. 76:503-506; Charpin, C., et al., (1991), J. Allergy Clin. Immunol. 88:77-82; Dabrowski, A. J., et al., (1990), J. Allergy Clin. Immunol. 86:462-465). It is also present in the salivary, perianal and lachrymal glands (Andersen, M. C., et al., (1985), J. Allergy Clin. Immunol. 76:563-569; van Milligen, F. J., et al., (1992), Int. Arch. Allergy Appl. Immunol. 92:375-378) and the principal reservoirs appear to be the skin and the fur (Mata, P., et al., (1992), Ann. Allergy 69(4):321-322).

The Fel d1 protein is of an unknown function to the animal but causes an IgG or IgE reaction in sensitive humans (either as an allergic or asthmatic response). Although other cat allergens are known, including Fel d2 (albumin) and Fel d3 (cystatin), 60% to 90% of the anti-cat IgE produced is directed against Fel d1 (Leitermann, K., et al., (1984), J Allergy Clin. Immunol. 74:147-153; Lowenstein, H., et al., (1985), Allergy 40:430-441; van Ree, R., et al., (1999), J. Allergy Clin. Immunol. 104:1223-1230; Ichikawa, K., et al., (2011), Clin. Exp. Allergy, 31:1279-1286).

Immunoglobulin E (IgE) is responsible for type 1 hypersensitivity, which manifests itself in allergic rhinitis, allergic conjunctivitis, hay fever, allergic asthma, bee venom allergy, and food allergies. IgE circulates in the blood and binds to high-affinity Fc receptors for IgE on basophils and mast cells. In most allergic responses, the allergens enter the body through inhalation, ingestion, or through the skin. The allergen then binds to preformed IgE already bound to the high affinity receptor on the surfaces of mast cells and basophils, resulting in cross-linking of several IgE molecules and triggering the release of histamine and other inflammatory mediators causing the various allergic symptoms.

The treatment for cat allergies includes desensitization therapy, which involves repeated injections with increasing dosages of either a crude cat dander extract, or short peptides derived from Fel d1. Using the crude extract of cat dander, Lilja, et. al., demonstrated that after three years of such treatment, patients allergic to cats still exhibited systemic symptoms (Lilja, Q., et al., (1989), J. Allergy Clin. Immunol. 83:37-44 and Hedlin, et al., (1991), J. Allergy Clin. Immunol. 87:955-964). Using short peptides derived from Fel d1 for desensitization resulted in a non-significant difference between the peptide group and the placebo control group (Oldfield, W. L., et al., (2002), Lancet, 360:47-53). Efficacy was only observed when large amounts (750 ug) of the short peptide were administered to patients (Norman, P. S., et al., (1996), Am. J. Respir. Crit. Care Med. 154:1623-1628). Furthermore, asthmatic reactions have been reported in patients given both crude extracts from cat dander, as well as in patients given short Fel d1 peptide treatment. Accordingly, there is a need in the field of cat allergy treatment for alternative strategies for treating patients sensitive to cat allergens, in particular Fel d1.

Antibodies have been proposed as a general treatment strategy for allergies, since they may be able to block the entry of allergenic molecules into the mucosal tissues, or may bind the allergen before it has the opportunity to bind to the IgE bound to the high affinity receptor on mast cells or basophils, thus preventing the release of histamine and other inflammatory mediators from these cells. U.S. Pat. No. 5,670,626 describes the use of monoclonal antibodies for the treatment of IgE-mediated allergic diseases such as allergic rhinitis, allergic asthma, and allergic conjunctivitis by blocking the binding of allergens to the mucosal tissue. U.S. Pat. No. 6,849,259 describes the use of allergen-specific antibodies to inhibit allergic inflammation in an in vivo mouse model of allergy. Milk-based and egg-based antibody systems have been described. For example, US20030003133A1 discloses using milk as a carrier for allergens for inducing oral tolerance to cat dander and other allergens. Compositions and methods for reducing an allergic response in an animal to an allergen in the environment through use of a molecule that inhibits the ability of the allergen to bind to mast cells was described in US2010/0143266. Other antibodies to Fel d1 were described by de Groot, et. al., (de Groot, et. al., (1988), J. Allergy Clin. Immunol. 82:778-786).

The fully human antibodies described herein demonstrate specific binding to Fel d1 and may be useful for treating patients suffering from cat allergies, in particular, in patients who demonstrate sensitivity to the Fel d1 allergen. The use of such antibodies may be an effective means of treating patients suffering from allergies to cat dander, or they may be used to prevent a heightened response to Fel d1 upon secondary exposure, or the accompanying symptoms associated with the allergy, or may be used to lessen the severity and/or the duration of the allergic response associated with a primary exposure to a cat harboring the Fel d1 allergen or with the recurrence of the symptoms upon secondary exposure. They may be used alone or as adjunct therapy with other therapeutic moieties or modalities known in the art for treating such allergies, such as, but not limited to, treatment with corticosteroids or epinephrine. They may be used in conjunction with a second or third different antibody specific for Fel d1. They may be used with allergen-specific immunotherapy (SIT).

In certain embodiments, the antibodies employed in the methods of the invention are obtained from mice immunized with a primary immunogen, such as natural Fel d1, which may be purchased commercially (See, for example, Indoor Biotech, #NA-FD1-2), or may be produced recombinantly. In certain embodiments, the immunogen may be either chain 1 of Fel d1, or chain 2 of Fel d1, or may be a combination of both chain 1 and chain 2 administered sequentially, or concurrently. The full-length amino acid sequence of chain 1 (also referred to as FELD1 A) is shown as SEQ ID NO: 392. Full-length amino acid sequences for chain 1 may also be found in GenBank accession numbers P30438 and NP_001041618.1. The full-length amino acid sequence of chain 2 (also referred to as FELD1 B) is shown as SEQ ID NO: 393. Full-length amino acid sequences for chain 2 may also be found in GenBank accession numbers PP30440 and NP_001041619.1.

In certain embodiments, the recombinantly produced Fel d1 immunogen may be made by direct fusion of the two chains of Fel d1, as described in Kaiser, et. al., to produce a fusion product that has a similar refolding pattern to that of natural Fel d1 (Kaiser, L., et al., (2003), J. Biol. Chem. 278(39):37730-37735). In certain embodiments, the immunogen may be a fusion protein such as that shown in the constructs of SEQ ID NOs: 385, 394, 395, 396 or 397, followed by immunization with a secondary immunogen, or with an immunogenically active fragment of the natural or recombinantly produced Fel d1.

The immunogen may be a biologically active and/or immunogenic fragment of natural or recombinantly produced Fel d1, or DNA encoding the active fragment thereof. The fragment may be derived from either the N-terminal or C-terminal of either chain 1 or chain 2, or from the N terminal or the C terminal of both chain 1 and chain 2. Fragments may be obtained from any site within chain 1 or chain 2 to be used as an immunogen for preparing antibodies to Fel d1.

In certain embodiments, the immunogen may be a fusion protein comprising any one or more of the following: i) amino acid residues 18-109 of chain 2 of Fel d1 (See GenBank accession number P30440 and also SEQ ID NO: 393) fused via the C terminus directly with the N terminus of amino acid residues 23-92 of chain 1 of Fel d1 (See GenBank accession number P30438 and also SEQ ID NO: 392); ii) amino acid residues 23-92 of chain 1 of Fel d1 (See GenBank accession number P30438 and also SEQ ID NO: 392) fused via the C terminus to the N terminus of amino acid residues 18-109 of chain 2 of Fel d1 (See GenBank accession number P30440 and also SEQ ID NO: 393); iii) amino acid residues 18-109 of chain 2 of Fel d1 (See GenBank accession number NP_001041619.1) fused via the C terminus directly with the N terminus of amino acid residues 19-88 of chain 1 of Fel d1 (See GenBank accession number NP_001041618.1), such as the construct shown in SEQ ID NO: 394 or 396; iv) amino acid residues 19-88 of chain 1 of Fel d1 (See GenBank accession number NP_001041618.1) fused via the C terminus to the N terminus of amino acid residues 18-109 of chain 2 of Fel d1 (See GenBank accession number NP_001041619.1). See also SEQ ID NO: 395. In certain embodiments, the fusion protein may have a tag at the C terminal end of the construct, such as a myc-myc-hexahistidine tag (See SEQ ID NOs: 385, 396 or 397 for such constructs.). In related embodiments, the fusion protein may have a mouse antibody Fc region coupled at the C terminal end of the construct (See SEQ ID NOs: 394 or 395 for such constructs.). In certain embodiments, chains 1 and 2 are coupled via a linker known to those skilled in the art, e.g., (G₄S)₃ (SEQ ID NO: 491) (See SEQ ID NOs: 395 and 397 for such a construct.).

In certain embodiments, antibodies that bind specifically to Fel d1 may be prepared using fragments of the above-noted regions, or peptides that extend beyond the designated regions by about 5 to about 20 amino acid residues from either, or both, the N or C terminal ends of the regions described herein. In certain embodiments, any combination of the above-noted regions or fragments thereof may be used in the preparation Fel d1 specific antibodies. In certain embodiments, any one or more of the above-noted regions of Fel d1, or fragments thereof may be used for preparing monospecific, bispecific, or multispecific antibodies.

### Antigen-Binding Fragments of Antibodies

Unless specifically indicated otherwise, the term “antibody,” as used herein, shall be understood to encompass antibody molecules comprising two immunoglobulin heavy chains and two immunoglobulin light chains (i.e., “full antibody molecules”) as well as antigen-binding fragments thereof. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. The terms “antigen-binding portion” of an antibody, or “antibody fragment”, as used herein, refers to one or more fragments of an antibody that retain the ability to specifically bind to either chain 1 and/or chain 2 of Fel d1. An antibody fragment may include a Fab fragment, a F(ab′) 2 fragment, a Fv fragment, a dAb fragment, a fragment containing a CDR, or an isolated CDR. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and (optionally) constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR, which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present invention include: (i) V_{H}-C_{H}1; (ii) V_{H}-C_{H}2; (iii) V_{H}-C_{H}3; (iv) V_{H}-C_{H}1-C_{H}2; (V) V_{H}-C_{H}1-C_{H}2-C_{H}3; (Vi) V_{H}-C_{H}2-C_{H}3; (Vii) V_{H}-C_{L}; (Viii) V_{L}-C_{H}1; (ix) V_{L}-C_{H}2; (X) V_{L}-C_{H}3; (xi) V_{L}-C_{H}1-C_{H}2; (XII) V_{L}-C_{H}1-C_{H}2-C_{H}3; (Xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids, which result in a flexible or semi-flexible linkage between adjacent variable and/or constant domains in a single polypeptide molecule. Moreover, an antigen-binding fragment of an antibody of the present invention may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

As with full antibody molecules, antigen-binding fragments may be mono-specific or multi-specific (e.g., bi-specific). A multi-specific antigen-binding fragment of an antibody will typically comprise at least two different variable domains, wherein each variable domain is capable of specifically binding to a separate antigen or to a different epitope on the same antigen. Any multi-specific antibody format, including the exemplary bi-specific antibody formats disclosed herein, may be adapted for use in the context of an antigen-binding fragment of an antibody of the present invention using routine techniques available in the art.

### Preparation of Human Antibodies

Methods for generating human antibodies in transgenic mice are known in the art. Any such known methods can be used in the context of the present invention to make human antibodies that specifically bind to Fel d1.

Using VELOCIMMUNE™ technology (see, for example, U.S. Pat. No. 6,596,541, Regeneron Pharmaceuticals, VELOCIMMUNE®) or any other known method for generating monoclonal antibodies, high affinity chimeric antibodies to Fel d1 are initially isolated having a human variable region and a mouse constant region. The VELOCIMMUNE® technology involves generation of a transgenic mouse having a genome comprising human heavy and light chain variable regions operably linked to endogenous mouse constant region loci such that the mouse produces an antibody comprising a human variable region and a mouse constant region in response to antigenic stimulation. The DNA encoding the variable regions of the heavy and light chains of the antibody are isolated and operably linked to DNA encoding the human heavy and light chain constant regions. The DNA is then expressed in a cell capable of expressing the fully human antibody.

Generally, a VELOCIMMUNE® mouse is challenged with the antigen of interest, and lymphatic cells (such as B-cells) are recovered from the mice that express antibodies. The lymphatic cells may be fused with a myeloma cell line to prepare immortal hybridoma cell lines, and such hybridoma cell lines are screened and selected to identify hybridoma cell lines that produce antibodies specific to the antigen of interest. DNA encoding the variable regions of the heavy chain and light chain may be isolated and linked to desirable isotypic constant regions of the heavy chain and light chain. Such an antibody protein may be produced in a cell, such as a CHO cell. Alternatively, DNA encoding the antigen-specific chimeric antibodies or the variable domains of the light and heavy chains may be isolated directly from antigen-specific lymphocytes.

Initially, high affinity chimeric antibodies are isolated having a human variable region and a mouse constant region. The antibodies are characterized and selected for desirable characteristics, including affinity, selectivity, epitope, etc. The mouse constant regions are replaced with a desired human constant region to generate the fully human antibody of the invention, for example wild-type or modified IgG1 or IgG4. While the constant region selected may vary according to specific use, high affinity antigen-binding and target specificity characteristics reside in the variable region.

In general, the antibodies used in the methods of the instant invention possess very high affinities, typically possessing K_{D} of from about 10⁻¹² through about 10⁻⁹ M, when measured by binding to antigen either immobilized on solid phase or in solution phase. The mouse constant regions are replaced with desired human constant regions to generate the fully human antibodies of the invention. While the constant region selected may vary according to specific use, high affinity antigen-binding and target specificity characteristics reside in the variable region. In some embodiments, the antibodies or antigen binding fragments thereof specifically bind monomeric Fel d1 with a K_{D} equal to or less than 1×10⁻⁸, 1×10⁻⁹, or 1×10⁻¹°. In some embodiments, the antibodies or antigen binding fragments thereof specifically bind dimeric Fel d1 with a K_{D} equal to or less than 1×10⁻⁸, 1×10⁻⁹, 1×10⁻¹⁰, or 1×10⁻¹¹. In embodiments, the antibodies or antigen binding fragments thereof specifically bind dimeric Fel d1 with a T_{1/2} of at least 150 min, 160 min, 170 min, 180 min, 190 min, 200 min, 210 min, 220 min, 230 min, 240 min, or 250 min. In embodiments, the antibodies or antigen binding fragments thereof specifically bind monomeric Fel d1 with a T_{1/2} of at least 25 min, 30 min, 35 min, 40 min, 45 min, or 50 min.

### Bioequivalents

The anti-Fel d1 antibodies and antibody fragments employed in the methods of the present invention encompass proteins having amino acid sequences that vary from those of the described antibodies, but that retain the ability to bind Fel d1. Such variant antibodies and antibody fragments comprise one or more additions, deletions, or substitutions of amino acids when compared to parent sequence, but exhibit biological activity that is essentially equivalent to that of the described antibodies. Likewise, the antibody-encoding DNA sequences described herein encompass sequences that comprise one or more additions, deletions, or substitutions of nucleotides when compared to the disclosed sequence, but that encode an antibody or antibody fragment that is essentially bioequivalent to an antibody or antibody fragment of the invention.

Two antigen-binding proteins, or antibodies, are considered bioequivalent if, for example, they are pharmaceutical equivalents or pharmaceutical alternatives whose rate and extent of absorption do not show a significant difference when administered at the same molar dose under similar experimental conditions, either single does or multiple dose. Some antibodies will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied.

In one embodiment, two antigen-binding proteins are bioequivalent, if there are no clinically meaningful differences in their safety, purity, and potency.

In one embodiment, two antigen-binding proteins are bioequivalent, if a patient can be switched one or more times between the reference product and the biological product without an expected increase in the risk of adverse effects, including a clinically significant change in immunogenicity, or diminished effectiveness, as compared to continued therapy without such switching.

In one embodiment, two antigen-binding proteins are bioequivalent if they both act by a common mechanism or mechanisms of action for the condition or conditions of use, to the extent that such mechanisms are known.

Bioequivalence may be demonstrated by in vivo and/or in vitro methods. Bioequivalence measures include, e.g., (a) an in vivo test in humans or other mammals, in which the concentration of the antibody or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the antibody (or its target) is measured as a function of time; and (d) in a well-controlled clinical trial that establishes safety, efficacy, or bioavailability or bioequivalence of an antibody.

Bioequivalent variants of the antibodies described herein may be constructed by, for example, making various substitutions of residues or sequences or deleting terminal or internal residues or sequences not needed for biological activity. For example, cysteine residues not essential for biological activity can be deleted or replaced with other amino acids to prevent formation of unnecessary or incorrect intramolecular disulfide bridges upon renaturation. In other contexts, bioequivalent antibodies may include antibody variants comprising amino acid changes, which modify the glycosylation characteristics of the antibodies, e.g., mutations that eliminate or remove glycosylation.

### Biological Characteristics of the Antibodies

In general, the antibodies employed in the methods of the present invention may function by binding to/interacting with either chain 1 or to chain 2 of Fel d1, or to both chain 1 and chain 2 of Fel d1 or to a fragment of either chain 1 or chain 2.

In certain embodiments, the antibodies may bind to an epitope located in at least the C-terminal region of either chain 1 or chain 2 of Fel d1. In one embodiment, the antibodies may bind to an epitope within the N-terminal region of either chain 1 or chain 2 of Fel d1.

In certain embodiments, the antibodies may function by blocking or inhibiting the binding of IgE to mast cells or basophils in a patient sensitive to the Fel d1 allergen.

In certain embodiments, the antibodies may function by binding to any other region or fragment of the full length chain 1 or chain 2 of the natural Fel d1 protein, the amino acid sequence of which is shown in SEQ ID NO: 392 (chain 1) and SEQ ID NO: 393 (chain 2).

In certain embodiments, the antibodies may be bi-specific antibodies. The bi-specific antibodies of the invention may bind one epitope in chain 1 and may also bind one epitope in chain 2. In certain embodiments, the bi-specific antibodies of the invention may bind two different epitopes in chain 1. In certain embodiments, the bi-specific antibodies of the invention may bind two different epitopes in chain 2. In certain embodiments, the bi-specific antibodies of the invention may bind to two different sites within the same helix on either one of chain 1 or chain 2, or may bind to the same helix on both chain 1 and chain 2. The structure of Fel d1 is described in greater detail in Kaiser, et. al. (Kaiser, L., et. al. (2003), J. Biol. Chem. 278 (39):37730-37735), whereby the authors note that Fel d1 consists of eight helices, H1-H4 and H5-H8, which correspond to chains 2 and 1, respectively, in natural Fel d1.

In one embodiment, the invention provides a method using at least one fully human monoclonal antibody or antigen-binding fragment thereof or combination of antibodies that binds to chain 1 and/or chain 2 of Fel d1, wherein each antibody or fragment thereof exhibits one or more of the following characteristics: (i) comprises a HCVR having an amino acid sequence selected from the group consisting of SEQ ID NO: 2, 18, 34, 50, 66, 82, 98, 114, 130, 146, 162, 178, 194, 210, 226, 242, 258, 274, 290, 306, 322, 338, 354 and 370, or a substantially similar sequence thereof having at least 90%, at least 95%, at least 98% or at least 99% sequence identity; (ii) comprises a LCVR having an amino acid sequence selected from the group consisting of SEQ ID NO: 10, 26, 42, 58, 74, 90, 106, 122, 138, 154, 170, 186, 202, 218, 234, 250, 266, 282, 298, 314, 330, 346, 362 and 378, or a substantially similar sequence thereof having at least 90%, at least 95%, at least 98% or at least 99% sequence identity; (iii) comprises a HCDR3 domain having an amino acid sequence selected from the group consisting of SEQ ID NO: 8, 24, 40, 56, 72, 88, 104, 120, 136, 152, 168, 184, 200, 216, 232,248, 264, 280, 296, 312, 328, 344, 360 and 376, or a substantially similar sequence thereof having at least 90%, at least 95%, at least 98% or at least 99% sequence identity; and a LCDR3 domain having an amino acid sequence selected from the group consisting of SEQ ID NO: 16, 32, 48, 64, 80, 96, 112, 128, 144, 160, 176, 192, 208, 224, 240, 256, 272, 288, 304, 320, 336, 352, 368 and 384, or a substantially similar sequence thereof having at least 90%, at least 95%, at least 98% or at least 99% sequence identity; (iv) comprises a HCDR1 domain having an amino acid sequence selected from the group consisting of SEQ ID NO: 4, 20, 36, 52, 68, 84, 100, 116, 132, 148, 164, 180, 196, 212, 228, 244, 260, 276, 292, 308, 324, 340, 356 and 372, or a substantially similar sequence thereof having at least 90%, at least 95%, at least 98% or at least 99% sequence identity; a HCDR2 domain having an amino acid sequence selected from the group consisting of SEQ ID NO: 6, 22, 38, 54, 70, 86, 102, 118, 134, 150, 166, 182, 198, 214, 230, 246, 262, 278, 294, 310, 326, 342, 358 and 374, or a substantially similar sequence thereof having at least 90%, at least 95%, at least 98% or at least 99% sequence identity; a LCDR1 domain having an amino acid sequence selected from the group consisting of SEQ ID NO: 12, 28, 44, 60, 76, 92, 108, 124, 140, 156, 172, 188, 204, 220, 236, 252, 268, 284, 300, 316, 332, 348, 364 and 380, or a substantially similar sequence thereof having at least 90%, at least 95%, at least 98% or at least 99% sequence identity; and a LCDR2 domain having an amino acid sequence selected from the group consisting of AAS, AAS, WAS, AAS, VVTS, KAS, DAS, KTS, DAS, RAS, AAS, GSS, YAS, YAS, AAS, YAS, YAS, AAS, YAS, KAS, GAS, TSS, AAS, and SAS, or a substantially similar sequence thereof having at least 90%, at least 95%, at least 98% or at least 99% sequence identity; (v) binds to chain 1 and/or chain 2 of Fel d1 with a K D equal to or less than 10⁻⁹; (vi) does not cross-react with, or bind to, uteroglobin; or (vii) blocks dye extravasation in vivo in a passive cutaneous anaphylaxis (PCA) mouse model using Fel d1 specific mouse IgE.

Non-limiting, exemplary in vitro assays for measuring binding activity are known in the art. The binding affinities and kinetic constants of human anti-Fel d1 antibodies can be determined by surface plasmon resonance, with the measurements conducted on a T200 Biacore instrument. Certain Fel d1 antibodies described herein, when used alone, or in combination, are able to bind to and neutralize at least one biological effect of Fel d1, as determined by in vitro or in vivo assays. The ability of the antibodies to bind to and neutralize the activity of Fel d1 may be measured using any standard method known to those skilled in the art, including binding assays, or neutralization of activity (e.g., protection from anaphylaxis) assays, as described herein.

In embodiments, one or more antibodies or antigen binding fragments thereof that inhibit Fel d1 induced basophil activation are selected that have an IC₅₀ of about 10 pM, 20 pM, 30 pM, 40 pM, 50 pM, 100 pM, 1 nM, or less. In an ELISA assay, one or more antibodies or antigen binding fragments thereof that inhibit Fel d1 binding to polyclonal IgE are selected that have an IC₅₀ of about 600 pM or less. One or more antibodies or antigen binding fragments thereof that inhibit Fel d1 binding to patient IgE are selected that have an IC₅₀ of about 500 pM or less.

The Fel d1 proteins or peptides may be modified to include addition or substitution of certain residues for tagging or for purposes of conjugation to carrier molecules, such as, KLH. For example, a cysteine may be added at either the N terminal or C terminal end of a peptide, or a linker sequence may be added to prepare the peptide for conjugation to, for example, KLH for immunization. The antibodies specific for Fel d1 may contain no additional labels or moieties, or they may contain an N-terminal or C-terminal label or moiety. In one embodiment, the label or moiety is biotin. In a binding assay, the location of a label (if any) may determine the orientation of the peptide relative to the surface upon which the peptide is bound. For example, if a surface is coated with avidin, a peptide containing an N-terminal biotin will be oriented such that the C-terminal portion of the peptide will be distal to the surface.

### Epitope Mapping and Related Technologies

The term “epitope,” as used herein, refers to an antigenic determinant that interacts with a specific antigen-binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstance, an epitope may include moieties of saccharides, phosphoryl groups, or sulfonyl groups on the antigen.

The present invention employs anti-Fel d1 antibodies that interact with one or more amino acids found within one or more regions of chain 1 or chain 2 of the Fel d1 molecule including, e.g., chain 1 (chain A) as shown in SEQ ID NO: 392, or chain 2 (chain B) as shown in SEQ ID NO: 393, or within comparable regions of a recombinantly produced Fel d1 protein, as shown in any one of SEQ ID NOs: 385, 394, 395, 396 or 397. The epitope to which the antibodies bind may consist of a single contiguous sequence of 3 or more (e.g., 3, 4, 5, 6, 7, 8, 9, 10, 11, 12, 13, 14, 15, 16, 17, 18, 19, 20 or more) amino acids located within any of the aforementioned regions or segments of the Fel d1 molecule (e.g., a linear epitope in either chain 1 or chain 2, or in a region that spans both chain 1 and chain 2). Alternatively, the epitope may consist of a plurality of non-contiguous amino acids (or amino acid sequences) located within either or both of the aforementioned regions or segments of the Fel d1 molecule (e.g., a conformational epitope).

Various techniques known to persons of ordinary skill in the art can be used to determine whether an antibody “interacts with one or more amino acids” within a polypeptide or protein. Exemplary techniques include, for example, routine cross-blocking assays, such as that described in Antibodies, Harlow and Lane (Cold Spring Harbor Press, Cold Spring Harb., NY). Other methods include alanine scanning mutational analysis, peptide blot analysis (Reineke, (2004) Methods Mol Biol 248:443-63), peptide cleavage analysis, crystallographic studies and NMR analysis. In addition, methods such as epitope excision, epitope extraction and chemical modification of antigens can be employed (Tomer, (2000) Protein Science 9: 487-496). Another method that can be used to identify the amino acids within a polypeptide with which an antibody interacts is hydrogen/deuterium exchange detected by mass spectrometry. In general terms, the hydrogen/deuterium exchange method involves deuterium-labeling the protein of interest, followed by binding the antibody to the deuterium-labeled protein. Next, the protein/antibody complex is transferred to water and exchangeable protons within amino acids that are protected by the antibody complex undergo deuterium-to-hydrogen back-exchange at a slower rate than exchangeable protons within amino acids that are not part of the interface. As a result, amino acids that form part of the protein/antibody interface may retain deuterium and therefore exhibit relatively higher mass compared to amino acids not included in the interface. After dissociation of the antibody, the target protein is subjected to protease cleavage and mass spectrometry analysis, thereby revealing the deuterium-labeled residues that correspond to the specific amino acids with which the antibody interacts. See, e.g., Ehring, (1999) Analytical Biochemistry 267(2):252-259; Engen and Smith, (2001) Anal. Chem. 73:256A-265A. X-ray crystallography of the antigen/antibody complex may also be used for epitope mapping purposes.

Modification-Assisted Profiling (MAP), also known as Antigen Structure-based Antibody Profiling (ASAP) is a method that categorizes large numbers of monoclonal antibodies (mAbs) directed against the same antigen according to the similarities of the binding profile of each antibody to chemically or enzymatically modified antigen surfaces (US 2004/0101920, herein specifically incorporated by reference in its entirety). Each category may reflect a unique epitope either distinctly different from or partially overlapping with epitope represented by another category. This technology allows rapid filtering of genetically identical antibodies, such that characterization can be focused on genetically distinct antibodies. When applied to hybridoma screening, MAP may facilitate identification of rare hybridoma clones that produce mAbs having the desired characteristics. MAP may be used to sort the antibodies of the invention into groups of antibodies binding different epitopes.

In certain embodiments, the epitope(s) for the antibodies used in the methods of the invention may be identified by either hydrogen deuterium exchange (HDX) or by X-ray crystallographic analysis.

The present invention also uses anti-Fel d1 antibodies that bind to the same epitope, or a portion of the epitope, as any of the specific exemplary antibodies described herein in Table 1, or an antibody having the CDR sequences of any of the exemplary antibodies described in Table 1. Likewise, the present invention also uses anti-Fel d1 antibodies that compete for binding to Fel d1 or a Fel d1 fragment with any of the specific exemplary antibodies described herein in Table 1, or an antibody having the CDR sequences of any of the exemplary antibodies described in Table 1.

One can easily determine whether an antibody binds to the same epitope as, or competes for binding with, a reference anti-Fel d1 antibody by using routine methods known in the art. For example, to determine if a test antibody binds to the same epitope as a reference anti-Fel d1 antibody of the invention, the reference antibody is allowed to bind to a Fel d1 protein or peptide under saturating conditions. Next, the ability of a test antibody to bind to the Fel d1 molecule is assessed. If the test antibody is able to bind to Fel d1 following saturation binding with the reference anti-Fel d1 antibody, it can be concluded that the test antibody binds to a different epitope than the reference anti-Fel d1 antibody. On the other hand, if the test antibody is not able to bind to the Fel d1 molecule following saturation binding with the reference anti-Fel d1 antibody, then the test antibody may bind to the same epitope as the epitope bound by the reference anti-Fel d1 antibody of the invention.

To determine if an antibody competes for binding with a reference anti-Fel d1 antibody, the above-described binding methodology is performed in two orientations: In a first orientation, the reference antibody is allowed to bind to a Fel d1 molecule under saturating conditions followed by assessment of binding of the test antibody to the Fel d1 molecule. In a second orientation, the test antibody is allowed to bind to a Fel d1 molecule under saturating conditions followed by assessment of binding of the reference antibody to the Fel d1 molecule. If, in both orientations, only the first (saturating) antibody is capable of binding to the Fel d1 molecule, then it is concluded that the test antibody and the reference antibody compete for binding to Fel d1. As will be appreciated by a person of ordinary skill in the art, an antibody that competes for binding with a reference antibody may not necessarily bind to the identical epitope as the reference antibody, but may sterically block binding of the reference antibody by binding an overlapping or adjacent epitope.

Two antibodies bind to the same or overlapping epitope if each competitively inhibits (blocks) binding of the other to the antigen. That is, a 1-, 5-, 10-, 20- or 100-fold excess of one antibody inhibits binding of the other by at least 50% but preferably 75%, 90% or even 99% as measured in a competitive binding assay (see, e.g., Junghans, et al., Cancer Res. 1990 50:1495-1502). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Additional routine experimentation (e.g., peptide mutation and binding analyses) can then be carried out to confirm whether the observed lack of binding of the test antibody is in fact due to binding to the same epitope as the reference antibody or if steric blocking (or another phenomenon) is responsible for the lack of observed binding. Experiments of this sort can be performed using ELISA, RIA, surface plasmon resonance, flow cytometry or any other quantitative or qualitative antibody-binding assay available in the art.

### Immunoconjugates

The invention encompasses methods using at least one human anti-Fel d1 monoclonal antibody conjugated to a therapeutic moiety (“immunoconjugate”), such as an agent that is capable of reducing the severity of an allergic response to the Fel d1 allergen present in cat dander or on cats, or in an area of the environment where cats may reside, or to ameliorate at least one symptom associated with exposure to cats, cat dander or to the Fel d1 allergen, including rhinitis, conjunctivitis, or breathing difficulties, or the severity thereof. Such an agent may be a corticosteroid, a second different antibody to Fel d1, or a vaccine. The type of therapeutic moiety that may be conjugated to the Fel d1 antibody will take into account the condition to be treated and the desired therapeutic effect to be achieved. Alternatively, if the desired therapeutic effect is to treat the sequelae or symptoms associated with exposure to the Fel d1 allergen, or any other condition resulting from such exposure, such as, but not limited to, rhinitis or conjunctivitis, it may be advantageous to conjugate an agent appropriate to treat the sequelae or symptoms of the condition, or to alleviate any side effects of the antibodies used herein. Examples of suitable agents for forming immunoconjugates are known in the art, see for example, WO 05/103081.

### Multi-Specific Antibodies

The antibodies employed in the methods of the present invention may be mono-specific, bi-specific, or multi-specific. Multi-specific antibodies may be specific for different epitopes of one target polypeptide or may contain antigen-binding domains specific for more than one target polypeptide. See, e.g., Tutt, et al., 1991, J. Immunol. 147:60-69; Kufer, et al., 2004, Trends Biotechnol. 22:238-244. The antibodies can be linked to or co-expressed with another functional molecule, e.g., another peptide or protein. For example, an antibody or fragment thereof can be functionally linked (e.g., by chemical coupling, genetic fusion, noncovalent association or otherwise) to one or more other molecular entities, such as another antibody or antibody fragment to produce a bi-specific or a multi-specific antibody with a second binding specificity. For example, the present invention may use bi-specific antibodies wherein one arm of an immunoglobulin may be specific for chain 1 of Fel d1, or a fragment thereof, and the other arm of the immunoglobulin may be specific for chain 2 of Fel d1, or a second therapeutic target, or may be conjugated to a therapeutic moiety.

### Antigen-Binding Fragments

An antigen-binding fragment of an antibody will typically comprise at least one variable domain. The variable domain may be of any size or amino acid composition and will generally comprise at least one CDR, which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of a bi-specific antigen-binding molecule may contain at least one variable domain covalently linked to at least one constant domain. Non-limiting, exemplary configurations of variable and constant domains that may be found within an antigen-binding domain of a bi-specific antigen-binding molecule may include: (i) V_{H}-C_{H}1; (ii) V_{H}-C_{H}2; (iii) V_{H}-C_{H}3; (iv) V_{H}-C_{H}1-C_{H}2; (V) V_{H}-C_{H}1-C_{H}2-C_{H}3; (Vi) V_{H}-C_{H}2-C_{H}3; (Vii) V_{H}-C_{L}; (Viii) V_{L}-C_{H}1; (ix) V_{L}-C_{H}2; (x) V_{L}-C_{H}3; (Xi) V_{L}-C_{H}1-C_{H}2; (XII) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids, which result in a flexible or semi-flexible linkage between adjacent variable and/or constant domains in a single polypeptide molecule. Moreover, an antigen-binding domain of a bi-specific antigen-binding molecule may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

The first antigen-binding domain and the second antigen-binding domain may be directly or indirectly connected to one another to form a bi-specific antigen-binding molecule. Alternatively, the first antigen-binding domain and the second antigen-binding domain may each be connected to a separate multimerizing domain. The association of one multimerizing domain with another multimerizing domain facilitates the association between the two antigen-binding domains, thereby forming a bispecific antigen-binding molecule. As used herein, a “multimerizing domain” is any macromolecule, protein, polypeptide, peptide, or amino acid that has the ability to associate with a second multimerizing domain of the same or similar structure or constitution. For example, a multimerizing domain may be a polypeptide comprising an immunoglobulin C_{H}3 domain. A non-limiting example of a multimerizing component is an Fc portion of an immunoglobulin, e.g., an Fc domain of an IgG selected from the isotypes IgG1, IgG2, IgG3, and IgG4, as well as any allotype within each isotype group. In certain embodiments, the multimerizing domain may be an Fc fragment or an amino acid sequence of 1 to about 200 amino acids in length containing at least one cysteine residues. In other embodiments, the multimerizing domain may be a cysteine residue, or a short cysteine-containing peptide. Other multimerizing domains include peptides or polypeptides comprising or consisting of a leucine zipper, a helix-loop motif, or a coiled-coil motif.

Any bi-specific antibody format or technology may be used to make bi-specific antigen-binding molecules. For example, an antibody or fragment thereof having a first antigen binding specificity can be functionally linked (e.g., by chemical coupling, genetic fusion, noncovalent association or otherwise) to one or more other molecular entities, such as another antibody or antibody fragment having a second antigen-binding specificity to produce a bi-specific antigen-binding molecule.

An exemplary bi-specific antibody format that can be used in the context of the present invention involves the use of a first immunoglobulin (Ig) CHs domain and a second Ig CHs domain, wherein the first and second Ig CHs domains differ from one another by at least one amino acid, and wherein at least one amino acid difference reduces binding of the bi-specific antibody to Protein A as compared to a bi-specific antibody lacking the amino acid difference. In one embodiment, the first Ig C_{H3} domain binds Protein A and the second Ig C_{H3} domain contains a mutation that reduces or abolishes Protein A binding such as an H95R modification (by IMGT exon numbering; H435R by EU numbering). The second C_{H3} may further comprise a Y96F modification (by IMGT; Y436F by EU). Further modifications that may be found within the second C_{H3} include: D16E, L18M, N44S, K52N, V57M, and V821 (by IMGT; D356E, L358M, N384S, K392N, V397M, and V4221 by EU) in the case of IgG1 antibodies; N44S, K52N, and V821 (IMGT; N384S, K392N, and V4221 by EU) in the case of IgG2 antibodies; and Q15R, N44S, K52N, V57M, R69K, E79Q, and V821 (by IMGT; Q355R, N384S, K392N, V397M, R409K, E419Q, and V4221 by EU) in the case of IgG4 antibodies. Variations on the bi-specific antibody format described above are contemplated within the scope of the present invention.

Other exemplary bi-specific formats that can be used in the context of the present invention include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED)body, leucine zipper, Duobody, IgG1/IgG2, dual acting Fab (DAF)-IgG, and Mabe bispecific formats (see, e.g., Klein, et al., 2012, mAbs 4:6, 1-11, and references cited therein, for a review of the foregoing formats). Bi-specific antibodies can also be constructed using peptide/nucleic acid conjugation, e.g., wherein unnatural amino acids with orthogonal chemical reactivity are used to generate site-specific antibody-oligonucleotide conjugates which then self-assemble into multimeric complexes with defined composition, valency and geometry. (See, e.g., Kazane, et al., J. Am. Chem. Soc. [Epub: Dec. 4, 2012]).

Selection of Allergen-Specific Antibodies

Any of the allergen-specific antibodies or antigen binding fragments thereof described herein can be evaluated for the ability to bind to and/or inhibit one or more actions of allergen-specific IgE, especially allergen-specific IgE obtained from a patient. Methods for treating comprising (a) collecting a sample of tissue or an extract thereof, or a biological fluid, or a blood sample from the patient; (b) extracting an allergen-specific IgE, or cells containing or bound to allergen-specific IgE, from one or more of the patient samples of (a); (c) mixing the IgE, or the cells containing or bound to IgE, from the patient sample with the allergen and with one or more antibodies or antigen binding fragments thereof specific for the allergen; (d) determining if the addition of one or more antibodies or antigen binding fragments thereof specific for the allergen blocks the binding of the allergen specific IgE from step (b) to the allergen, and if the results from step (d) demonstrate effective blocking of the binding of the allergen specific IgE to the allergen with the allergen specific antibodies of step (c), administering to the patient a pharmaceutical composition comprising a therapeutically effective amount of one or more of the allergen specific antibodies or antigen binding fragments from step (c). are described in WO2018/118713A1.

### Therapeutic Administration and Formulations

The invention provides therapeutic compositions comprising the anti-Fel d1 antibodies or antigen-binding fragments thereof employed in the methods disclosed herein. The phrases “therapeutic composition” and “pharmaceutical composition” are used interchangeably herein. The administration of therapeutic compositions in accordance with the invention will be administered via a suitable route including, but not limited to, intravenously, subcutaneously, intramuscularly, intranasally, with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell, et al., “Compendium of excipients for parenteral formulations” PDA (1998) J Pharm Sci Technol 52:238-311.

The dose of each antibody may vary depending upon the age and the size of a subject to be administered, target disease, conditions, route of administration, and the like. In certain embodiments, each antibody or antigen-binding fragment can be administered as an initial dose of at least about 0.1 mg to about 800 mg, about 1 to about 600 mg, about 5 to about 300 mg, or about 10 to about 200 mg, to about 100 mg, or to about 50 mg. In certain embodiments, the initial dose may be followed by administration of a second or a plurality of subsequent doses of the antibody or antigen-binding fragment thereof in an amount that can be approximately the same or less than that of the initial dose, wherein the subsequent doses are separated by at least 1 day to 3 days; at least one week, at least 2 weeks; at least 3 weeks; at least 4 weeks; at least 5 weeks; at least 6 weeks; at least 7 weeks; at least 8 weeks; at least 9 weeks; at least 10 weeks; at least 12 weeks; or at least 14 weeks.

Various delivery systems are known and can be used to administer the pharmaceutical composition of the invention, e.g., encapsulation in liposomes, microparticles, microcapsules, recombinant cells capable of expressing the mutant viruses, receptor mediated endocytosis (see, e.g., Wu, et al., (1987) J. Biol. Chem. 262:4429-4432). Methods of introduction include, but are not limited to, intradermal, transdermal, intramuscular, intraperitoneal, intravenous, subcutaneous, intranasal, epidural and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

The pharmaceutical composition can be also delivered in a vesicle, in particular a liposome (see, for example, Langer, (1990) Science 249:1527-1533).

In certain situations, the pharmaceutical composition can be delivered in a controlled release system. In one embodiment, a pump may be used. In another embodiment, polymeric materials can be used. In yet another embodiment, a controlled release system can be placed in proximity of the composition's target, thus requiring only a fraction of the systemic dose.

The injectable preparations may include dosage forms for intravenous, subcutaneous, intracutaneous and intramuscular injections, drip infusions, etc. These injectable preparations may be prepared by methods publicly known. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

A pharmaceutical composition of the present invention can be delivered subcutaneously or intravenously with a standard needle and syringe. In addition, with respect to subcutaneous delivery, a pen delivery device readily has applications in delivering a pharmaceutical composition of the present invention. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition of the present invention. Examples include, but certainly are not limited to AUTOPEN™ (Owen Mumford, Inc., Woodstock, UK), DISETRONIC™ pen (Disetronic Medical Systems, Burghdorf, Switzerland), HUMALOG MIX 75/25™ pen, HUMALOG™ pen, HUMALIN 70/30™ pen (Eli Lilly and Co., Indianapolis, IN), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™, OPTIPEN PRO™, OPTIPEN STARLET™, and OPTICLIK™ (sanofi-aventis, Frankfurt, Germany), to name only a few. Examples of disposable pen delivery devices having applications in subcutaneous delivery of a pharmaceutical composition of the present invention include, but certainly are not limited to the SOLOSTAR™ pen (sanofi-aventis), the FLEXPEN™ (Novo Nordisk), and the KWIKPEN™ (Eli Lilly), the SURECLICK™ Autoinjector (Amgen, Thousands Oaks, CA), the PENLET™ (Haselmeier, Stuttgart, Germany), the EPI PEN (Dey, L.P.) and the HUMIRA™ Pen (Abbott Labs, Abbott Park, Ill.), to name only a few.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. The amount of the aforesaid antibody contained is generally about 5 to about 500 mg per dosage form in a unit dose; especially in the form of injection, it is preferred that the aforesaid at least one antibody is contained in about 5 to about 100 mg and in about 10 to about 250 mg for the other dosage forms.

### Therapeutic Uses of the Antibodies

Due to their interaction with Fel d1, the antibodies described herein are useful for treating the primary response following exposure of an individual to a cat, cat dander or to an environment containing the Fel d1 protein, or at least one symptom associated with the allergic response, such as itchy eyes, conjunctivitis, rhinitis, wheezing, breathing difficulties, or for preventing a secondary response to the Fel d1 allergen, including a more serious anaphylactic response, or for lessening the severity, duration, and/or frequency of symptoms following reexposure to the cat allergen. It is herein envisioned that the antibodies may be used prophylactically.

In one aspect, the antibodies described herein are useful for reducing the severity, duration, or frequency of occurrence of one or more symptoms associated with an allergic response in a patient, wherein the allergic response is the result of exposure to an animal product.

In another aspect, the antibodies described herein are useful for increasing the time to early asthmatic response (EAR) upon exposure to an animal product in a patient having an allergy to the animal product

In another aspect, the antibodies described herein are useful for increasing the quantity of an animal product tolerated by a patient having an allergy to the animal product.

In another aspect, the antibodies described herein are useful for improving lung function upon exposure to an animal product in a patient having an allergy to the animal product. Putative patients may have a cat allergy and/or mild asthma.

In embodiments, measurements are conducted prior to therapy, and post administration of the one or more allergen specific monoclonal antibodies or antigen binding fragments thereof to the patient upon exposure to the allergen. In embodiments, measurements are conducted at one or more time points post administration such as at one week post administration, at one month post administration, at two months post administration, at three months post administration, at 6 months post administration, at 12 months post administration, and/or at 24 months post administration. If the measurements show that post administration of the allergen-specific monoclonal antibodies or antigen binding fragments thereof to the patient the severity, duration, or frequency of occurrence of one or more symptoms associated with an allergic response in a patient is decreased as compared to the measurements prior to therapy, treatment is continued or may be discontinued until symptoms return to pre administration levels. If the measurements show that post administration of the allergen-specific monoclonal antibodies or antigen binding fragments thereof to the patient the severity, duration, or frequency of occurrence of one or more symptoms associated with an allergic response in a patient is not decreased as compared to the measurements prior to therapy, treatment may be discontinued, the dosage of the allergen specific antibody or antigen binding fragment thereof may be changed, or a different antibody or antigen binding fragment or combinations thereof may be utilized.

In one embodiment, the one or more symptoms may be selected from the group consisting of nasal congestion, nasal itching, rhinorrhea and sneezing. In one embodiment, the patient reported outcome score is selected from the group consisting of a Total Nasal Symptom Score (TNSS), a Visual Analog Scale (VAS) nasal symptoms score and an improvement in peak nasal inspiratory flow (PNIF). In one embodiment, the method is associated with a reduction in wheal diameter in an allergen skin test. In one embodiment, the allergen skin test is a titrated skin prick test (SPT), wherein there is a significant reduction in wheal diameter in a patient treated with the allergen-specific monoclonal antibodies compared to the wheal diameter observed prior to treatment of the patient with the allergen-specific monoclonal antibodies.

In embodiments, the treatment is monitored by a determination of one or more efficacy endpoints. An example of an efficacy endpoint is to determine the delay/prevention of an allergen challenge-induced early asthmatic response (EAR), and/or to assess improvement in lung function by measures of FEV1 compared to placebo-treated patients on day 8. Other efficacy endpoints include allergic rhinitis symptoms assessed by total nasal symptom score (TNSS) compared to placebo patients on days 8, 29, 57, and 85, and ocular symptoms assessed by total ocular symptom score (TOSS) compared to placebo patients on days 8, 29, 57, and 85.

Another example of an efficacy endpoint is to determine if one can increase the exposure to cat allergen, measured as a product of minute ventilation and time, required to induce EAR in a Controlled Cat Allergen Challenge (40 ng/m³ Fel d1 allergen×minute ventilation×time) as compared to placebo patients on days 8, 29, 57, and 85.

The evaluation of the safety and tolerability of REGN1908-1909 vs. placebo in patients with cat allergen-triggered asthma is another efficacy endpoint.

### Combination Therapies

Combination therapies may include at least one anti-Fel d1 antibody described herein and any additional therapeutic agent that may be advantageously combined with the same.

For example, a second therapeutic agent may be employed to aid in reducing the allergic symptoms following exposure to a cat, cat dander, cat hair or an extract thereof, or Fel d1, or being exposed to an environment in which a cat resides, such as a corticosteroid. The antibodies may also be used in conjunction with other therapies, such as a vaccine specific for the Fel d1 allergen. The additional therapeutically active component(s) may be administered prior to, concurrent with, or after the administration of the at least one anti-Fel d1 antibody used herein. For purposes of the present disclosure, such administration regimens are considered the administration of at least one anti-Fel d1 antibody “in combination with” a second therapeutically active component.

### Administration Regimens

According to certain embodiments of the present invention, multiple doses of one or more anti-Fel d1 antibodies (an antibody combination) or a bi-specific antigen-binding molecule may be administered to a subject over a defined time course. The methods according to this aspect of the invention may comprise sequentially administering to a subject multiple doses of an antibody, antibody combination, or a bi-specific antigen-binding molecule of the invention. As used herein, “sequentially administering” means that each dose of an antibody, antibody combination, or a bi-specific antigen-binding molecule is administered to the subject at a different point in time, e.g., on different days separated by a predetermined interval (e.g., hours, days, weeks or months). The present invention includes methods, which may comprise sequentially administering to the patient a single initial dose of an antibody, antibody combination, or a bi-specific antigen-binding molecule, followed by one or more secondary doses of the antibody, and optionally followed by one or more tertiary doses of the antibody.

The terms “initial dose,” “secondary doses,” and “tertiary doses,” refer to the temporal sequence of administration of an antibody, antibody combination, or a bi-specific antigen-binding molecule described herein. Thus, the “initial dose” is the dose administered at the beginning of the treatment regimen (also referred to as the “baseline dose”); the “secondary doses” are the doses that are administered after the initial dose; and the “tertiary doses” are the doses that are administered after the secondary doses. The initial, secondary, and tertiary doses may all contain the same amount of an antibody, antibody combination, or a bi-specific antigen-binding molecule, but generally may differ from one another in terms of frequency of administration. In certain embodiments, however, the amount of an antibody, antibody combination, or a bi-specific antigen-binding molecule contained in the initial, secondary and/or tertiary doses varies from one another (e.g., adjusted up or down as appropriate) during the course of treatment. In certain embodiments, two or more (e.g., 2, 3, 4, or 5) doses are administered at the beginning of the treatment regimen as “loading doses” followed by subsequent doses that are administered on a less frequent basis (e.g., “maintenance doses”).

In one exemplary embodiment of the present invention, each secondary and/or tertiary dose is administered 1 to 26 (e.g., 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, 15, 15½, 16, 16½, 17, 17½, 18, 18½, 19, 19½, 20, 20½, 21, 21½, 22, 22½, 23, 23½, 24, 24½, 25, 25½, 26, 26½, or more) weeks after the immediately preceding dose. The phrase “the immediately preceding dose,” as used herein, means, in a sequence of multiple administrations, the dose of an antibody, antibody combination, or a bi-specific antigen-binding molecule, which is administered to a patient prior to the administration of the very next dose in the sequence with no intervening doses.

The methods according to this aspect of the invention may comprise administering to a patient any number of secondary and/or tertiary doses of an antibody, antibody combination, or a bi-specific antigen-binding molecule that specifically binds Fel d1. For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In embodiments involving multiple secondary doses, each secondary dose may be administered at the same frequency as the other secondary doses. For example, each secondary dose may be administered to the patient 1 to 2 weeks after the immediately preceding dose. Similarly, in embodiments involving multiple tertiary doses, each tertiary dose may be administered at the same frequency as the other tertiary doses. For example, each tertiary dose may be administered to the patient 2 to 4 weeks after the immediately preceding dose. Alternatively, the frequency at which the secondary and/or tertiary doses are administered to a patient can vary over the course of the treatment regimen. The frequency of administration may also be adjusted during the course of treatment by a physician depending on the needs of the individual patient following clinical examination.

In embodiments, one or more doses of an allergen specific antibody or antigen binding fragment thereof is administered depending on whether the severity, duration, and/or frequency of occurrence of one or more symptoms associated with an allergic response in a patient post administration are decreased or increased as compared to the severity, duration, and/or frequency of occurrence of one or more symptoms associated with an allergic response in a patient prior to administration. In certain embodiments, if the patient exhibits at least a 30%, at least 40%, or at least 50% decrease in the severity, duration, and/or frequency of occurrence of one or more symptoms associated with an allergic response in a patient at a measured time point post administration as compared to a measurement prior administration (i.e., baseline score), no additional doses of the allergen specific antibodies may be necessary. In other embodiments, if the patient exhibits a reduction in the area under the curve (AUC) for TNSS of at least 30%, at least 40%, at least 50% or at least 60%, at a measured time point post administration as compared to the AUC for TNSS prior to administration (i.e., baseline score), no further doses may be necessary.

In embodiments, an allergen specific antibody or antigen binding fragment thereof is administered once a month, once every two months, once every three months, once every 6 months, or once very year. In embodiments, administration frequency is determined by whether the patient exhibits at least a 30%, at least 40%, or at least 50% decrease in the severity, duration, and/or frequency of occurrence of one or more symptoms associated with an allergic response in a patient at a measured time point post administration as compared to a measurement prior to administration (i.e., baseline score). If the severity, duration, and/or frequency of occurrence of one or more symptoms associated with an allergic response is decreased more than 50%, the patient may no longer need administration of the allergen specific antibody or antigen binding fragment thereof. If the symptoms are not reduced or recur after a reduction, administration of the allergen specific antibody or antigen binding fragment thereof may resume until the desired level of symptom reduction is obtained.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1. Generation of Human Antibodies to Fel d1

The following generation of human antibodies to Fel d1 was disclosed in WO2018/118713A1. An immunogen comprising any one of the following can be used to generate antibodies to Fel d1. In certain embodiments, the antibodies employed in the compositions and methods according to the invention are obtained from mice immunized with a primary immunogen, such as full length natural Fel d1 (nFel d1), which may be purchased commercially (e.g., from Indoor Biotechnologies, #LTN-FD1-1), or isolated from cat hair or dander by multi-step column chromatography (See, for example, Chapman M D, et al., (1988), J. Immunol. 140:812-818), or which may be produced recombinantly (See GenBank accession numbers P30438, or NP_001041618.1 for the full length amino acid sequence of chain 1 of Fel d1 (also referred to as chain A or FELD1 A; also see SEQ ID NO: 392) and GenBank accession number P30440, or NP_001041619.1 for the full length amino acid sequence of chain 2 of Fel d1 (also referred to as chain B or FELD B; also see SEQ ID NO: 393), or fragments of either chain 1 or chain 2, or fragments from both chain 1 and chain 2 of the Fel d1 protein, followed by immunization with a secondary immunogen, or with an immunogenically active fragment of the natural protein. Animals may be immunized with either chain 1 protein alone or chain 2 protein alone, or with both chain 1 and chain 2 proteins, administered sequentially, or concurrently. Various constructs may be prepared using portions of chain 1 and chain 2 along with various linking or spacer strategies known to those skilled in the art. These constructs may be used alone, or in various combinations to elicit antibody responses in vivo. For example, recombinant Fel d1 constructs, such as those exemplified in SEQ ID NOs: 385, 394, 395, 396 or 397, or fragments thereof, may be used as immunogens.

In certain embodiments, the antibodies employed in the compositions and methods according to the invention are obtained from mice immunized with a primary immunogen, such as a biologically active and/or immunogenic fragment of natural Fel d1, or DNA encoding the active fragment thereof. The fragment may be derived from the N-terminal or C-terminal domain of either chain 1 and/or chain 2 of Fel d1.

In certain embodiments, the recombinantly produced Fel d1 immunogen may be made by direct fusion of the two chains of Fel d1, as described in Kaiser, et. al., to produce a fusion product that has a similar refolding pattern to that of natural Fel d1 (Kaiser, L., et al., (2003), J. Biol. Chem. 278(39):37730-37735). In certain embodiments, the immunogen may be a fusion protein such as that shown in the constructs of SEQ ID NOs: 385, 394, 395, 396 or 397, followed by immunization with a secondary immunogen, or with an immunogenically active fragment of the natural or recombinantly produced Fel d1.

In certain embodiments, the recombinant Fel d1 protein constructs used in the studies described herein are comprised of either i) Fel d1 B chain (chain 2) and Fel d1 A chain (chain 1) linked as a continuous, in-line fusion (with Fel d1 B chain at the N-terminus) or ii) a continuous, in-line fusion with Fel d1 A chain at the N-terminus followed by a flexible linker [(Gly4Ser)₃ (SEQ ID NO: 491)] followed by Fel d1 B. These constructs may also include a C-terminal tag (myc-myc-His6 or mouse IgG2a Fc region), as indicated below. The proteins were expressed in Chinese hamster ovary (CHO) cells. An exogenous signal sequence used to promote expression in CHO cells is not included in the sequence listings.

In certain embodiments, the immunogen may be a fusion protein comprising any one or more of the following: i) amino acid residues 18-109 of chain 2 of Fel d1 (See GenBank accession number P30440 and also SEQ ID NO: 393) fused via the C terminus directly with the N terminus of amino acid residues 23-92 of chain 1 of Fel d1 (See GenBank accession number P30438 and also SEQ ID NO: 392); ii) amino acid residues 23-92 of chain 1 of Fel d1 (See GenBank accession number P30438 and also SEQ ID NO: 392) fused via the C terminus to the N terminus of amino acid residues 18-109 of chain 2 of Fel d1 (See GenBank accession number P30440 and also SEQ ID NO: 393); iii) amino acid residues 18-109 of chain 2 of Fel d1 (See GenBank accession number NP_001041619.1) fused via the C terminus directly with the N terminus of amino acid residues 19-88 of chain 1 of Fel d1 (See GenBank accession number NP_001041618.), such as the construct shown in SEQ ID NO: 394 or 396; iv) amino acid residues 19-88 of chain 1 of Fel d1 (See GenBank accession number NP_001041618.1) fused via the C terminus to the N terminus of amino acid residues 18-109 of chain 2 of Fel d1 (See GenBank accession number NP_001041619.1). See also SEQ ID NO: 395). In certain embodiments, the fusion protein may have a tag at the C terminal end of the construct, such as a myc-myc-hexahistidine tag (See SEQ ID NOs: 385, 396 or 397 for such constructs.). In related embodiments, the fusion protein may have a mouse Fc coupled at the C terminal end of the construct (See SEQ ID NOs: 394 or 395 for such constructs.). In certain embodiments, chains 1 and 2 are coupled via a linker known to those skilled in the art, e.g., (G₄S)₃ (SEQ ID NO: 491) (See SEQ ID NOs: 395 and 397 for such a construct.).

In certain embodiments, antibodies that bind specifically to Fel d1 may be prepared using fragments of the above-noted regions, or peptides that extend beyond the designated regions by about 5 to about 20 amino acid residues from either, or both, the N or C terminal ends of the regions described herein. In certain embodiments, any combination of the above-noted regions or fragments thereof may be used in the preparation of Fel d1 specific antibodies. In certain embodiments, any one or more of the above-noted regions of Fel d1, or fragments thereof may be used for preparing monospecific, bispecific, or multispecific antibodies.

The full-length proteins, or fragments thereof, that were used as immunogens, as noted above, were administered directly, with an adjuvant to stimulate the immune response, to a VELOCIMMUNE® mouse comprising DNA encoding human Immunoglobulin heavy and kappa light chain variable regions. The antibody immune response was monitored by a Fel d1-specific immunoassay. When a desired immune response was achieved splenocytes were harvested and fused with mouse myeloma cells to preserve their viability and form hybridoma cell lines. The hybridoma cell lines were screened and selected to identify cell lines that produce Fel d1 specific antibodies. Using this technique, and the various immunogens described above, several anti-Fel d1, chimeric antibodies (i.e., antibodies possessing human variable domains and mouse constant domains) were obtained; certain exemplary antibodies generated in this manner were designated as H1M1230N, H1M1234N, H1M1241N, H2M1233N, H2M1236N, H2M1237N, and H2M1242N.

Anti-Fel d1 antibodies were also isolated directly from antigen-positive B cells without fusion to myeloma cells, as described in U.S. 2007/0280945A1, herein specifically incorporated by reference in its entirety. Using this method, several fully human anti-Fel d1 antibodies (i.e., antibodies possessing human variable domains and human constant domains) were obtained; exemplary antibodies generated in this manner were designated as follows: H4H2574P, H4H2590S, H4H2592B, H4H2594S, H4H2597P, H4H2606B, H4H2607B, H4H2608B, H4H2636P, H4H2645P, H4H2793P, H4H2797P and H4H2864P.

### Example 2. Heavy and Light Chain Variable Region Amino Acid Sequences

Table 1 sets forth the heavy and light chain variable region amino acid sequence pairs of selected antibodies specific for Fel d1 and their corresponding antibody identifiers. Antibodies are typically referred to herein according to the following nomenclature: Fc prefix (e.g., “H4H”, “H1M, “H2M”), followed by a numerical identifier (e.g., “1232” as shown in Table 1), followed by a “P” or “N” suffix. Thus, according to this nomenclature, an antibody may be referred to as, e.g. “H1M1232N”. The H4H, H1 M, and H2M prefixes on the antibody designations used herein indicate the particular Fc region of the antibody. For example, an “H2M” antibody has a mouse IgG2 Fc, whereas an “H4H” antibody has a human IgG4 Fc. As will be appreciated by a person of ordinary skill in the art, an H1M or H2M antibody can be converted to an H4H antibody, and vice versa, but in any event, the variable domains (including the CDRs), which are indicated by the numerical identifiers shown in Table 1, will remain the same. Antibodies having the same numerical antibody designation, but differing by a letter suffix of N, B, S or P refer to antibodies having heavy and light chains with identical CDR sequences but with sequence variations in regions that fall outside of the CDR sequences (i.e., in the framework regions). Thus, N, B, S and P variants of a particular antibody have identical CDR sequences within their heavy and light chain variable regions but differ from one another within their framework regions.

TABLE 1

| | AMINO ACID SEQ ID NOs: | | | | | | | |
|---|---|---|---|---|---|---|---|---|
| Antibody | | HCDR | HCDR | HCDR | | LCDR | LCDR | LCDR |
| Designation | HCVR | 1 | 2 | 3 | LCVR | 1 | 2 | 3 |
| H1M1230N | 2 | 4 | 6 | 8 | 10 | 12 | AAS | 16 |
| H4H1232N | 18 | 20 | 22 | 24 | 26 | 28 | AAS | 32 |
| H1M1234N | 34 | 36 | 38 | 40 | 42 | 44 | WAS | 48 |
| H1M1241N | 50 | 52 | 54 | 56 | 58 | 60 | AAS | 64 |
| H4H1300N | 66 | 68 | 70 | 72 | 74 | 76 | WTS | 80 |
| H2M1233N | 82 | 84 | 86 | 88 | 90 | 92 | KAS | 96 |
| H2M1236N | 98 | 100 | 102 | 104 | 106 | 108 | DAS | 112 |
| H2M1237N | 114 | 116 | 118 | 120 | 122 | 124 | KTS | 128 |
| H4H1238N | 130 | 132 | 134 | 136 | 138 | 140 | DAS | 144 |
| H2M1242N | 146 | 148 | 150 | 152 | 154 | 156 | RAS | 160 |
| H4H1616N | 162 | 164 | 166 | 168 | 170 | 172 | AAS | 176 |
| H4H2574P | 178 | 180 | 182 | 184 | 186 | 188 | GSS | 192 |
| H4H2590S | 194 | 196 | 198 | 200 | 202 | 204 | YAS | 208 |
| H4H2592B | 210 | 212 | 214 | 216 | 218 | 220 | YAS | 224 |
| H4H2594S | 226 | 228 | 230 | 232 | 234 | 236 | AAS | 240 |
| H4H2597P | 242 | 244 | 246 | 248 | 250 | 252 | YAS | 256 |
| H4H2606B | 258 | 260 | 262 | 264 | 266 | 268 | YAS | 272 |
| H4H2607B | 274 | 276 | 278 | 280 | 282 | 284 | AAS | 288 |
| H4H2608B | 290 | 292 | 294 | 296 | 298 | 300 | YAS | 304 |
| H4H2636P | 306 | 308 | 310 | 312 | 314 | 316 | KAS | 320 |
| H4H2645P | 322 | 324 | 326 | 328 | 330 | 332 | GAS | 336 |
| H4H2793P | 338 | 340 | 342 | 344 | 346 | 348 | TSS | 352 |
| H4H2797P | 354 | 356 | 358 | 360 | 362 | 364 | AAS | 368 |
| H4H2864P | 370 | 372 | 374 | 376 | 378 | 380 | SAS | 384 |
| H4H2574B | 428 | 430 | 432 | 434 | 436 | 438 | WAS | 442 |
| H4H2597B | 444 | 446 | 448 | 450 | 452 | 454 | YAS | 458 |
| H4H2636B | 460 | 462 | 464 | 466 | 468 | 470 | KAS | 474 |
| H4H2645B | 476 | 478 | 480 | 482 | 484 | 486 | GAS | 490 |

Bi-specific antibodies comprising heavy and light chain binding domains from pairs of certain of the anti-Fel d1 antibodies described above were constructed using standard methodologies, as disclosed in WO2018/118713A1.

### Example 3: Generation of REGN1908 (H4H1232N) and REGN1909 (H4H2636P)

REGN1908 and REGN1909 were produced as described in Example 1, above. REGN1908 is also referred to as H4H1232N and comprises a heavy chain variable region (HCVR) amino acid sequence of SEQ D NO: 18 and a light chain variable region (LCVR) amino acid sequence of SEQ ID NO: 26. REGN1908 also has the following heavy and light chain complementarity determining region (HCDRs and LCDRs, respectively) amino acid sequences: HCDR1, 2 and 3: SEQ ID NOs: 20, 22 and 24; LCDR1, 2 and 3: SEQ ID NOs: 28, AAS, and SEQ ID NO: 32. REGN1909 is also referred to as H4H2636P and comprises a heavy chain variable region (HCVR) amino acid sequence of SEQ D NO:306 and a light chain variable region (LCVR) amino acid sequence of SEQ ID NO: 314. REGN1909 also has the following heavy and light chain complementarity determining region (HCDRs and LCDRs, respectively) amino acid sequences: HCDR1, 2 and 3: SEQ ID NOs: 308, 310 and 312; LCDR1, 2 and 3: SEQ ID NOs: 316, KAS, and SEQ ID NO: 320. Both natural Fel d1 (nFel d1; obtained from Indoor Biotech) and recombinant Fel d1 (rFel d1) were used in in vitro assays. Recombinant Fel d1 was produced following the design of Kaiser, et al., (Kaiser, L., et al., (2003), The Journal of Biological Chemistry 278 (39): 37730-35) who showed that single-chain fusions were structurally and functionally equivalent to the natural Fel d1 heterodimer. The Regeneron-produced recombinant proteins include amino acids 18-109 of Fel d1 Chain 2 (NP_001041619.1) at the N-terminus fused directly in-line to amino acids 23-92 of Fel d1 Chain 1 (NP_001041618.1) with a D27G mutation and a C-term Myc-Myc-6×His tag. The proteins were expressed in Chinese Hamster Ovary (CHO) cells and made with either a monomeric (myc-myc-hexahistidine) or a dimeric (mouse IgG2a Fc) C-terminal tag (rFel d1-mmH and rFel d1-mFc, respectively). The dimeric Fc fusion Fc_{ε}R1α was generated to support development of the ELISA-based competition assay.

### Example 4: REGN1908-1909 Significantly Reduces Clinical Symptoms in Response to Nasal Allergen Challenge in Cat-Allergic Human Subjects: A Single Dose Study of the Efficacy, Safety, Tolerability and Pharmacokinetics of REGN1908-1909 in Allergic Rhinitis Subjects Challenged Intranasally with Allergen Extract

### Clinical Study Design:

A multicenter phase 1b, randomized, double-blind, placebo-controlled, single subcutaneous dose, proof-of-mechanism study was conducted in 6 study centers in Europe and Asia-Pacific, as described in WO2018/118713A1, incorporated herein by reference. Subjects eligible for randomization were randomized 1:1 on day 1 to receive a single SC dose of REGN1908-1909 (600 mg total, 1:1 antibody ratio; n=37) or placebo (n=36).

### Patient Population:

Eligible subjects were 18-55 years of age with cat-induced allergic rhinitis and cat sensitization confirmed at screening. To confirm cat-sensitization, subjects underwent screening at two visits, day −28 and day −14 (+/−2 days). At screening visit 1, subjects were screened for allergen-specific IgE, underwent a skin prick test with cat hair extract (cat-SPT, Aquagen©, ALK-Abello) and other allergen extracts, and were tested for lung function (FEV1). Subjects were eligible for screening visit 2 based on IgE titers specific for Fel d1 and cat hair extract ≥0.35 kAU/l each, cat-SPT mean wheal diameter of ≥3 mm compared to a negative control SPT, and normal lung function. Subjects were excluded if they had prior history of SIT or vaccination with cat allergen, or anti-IgE therapy; SIT to other allergens within 3 months prior to screening; or were living with or chronically exposed to a cat.

At screening visit 2, a single nasal allergen challenge (NAC) was performed using increasing doses of cat hair extracts (100-33,000 SQ-U/ml). Briefly, cat hair extract was applied intranasally every 10 min for 1 hour, or until a total nasal symptom score (TNSS) ≥7 was reached. TNSS (measured on a 0-12 scale) is a composite patient symptom assessment of congestion, itching, and rhinorrhea (each graded on 0-3 scale, 3 being severe), and sneezing (3 being >5 sneezes). Cat-sensitized subjects were eligible for enrollment based on having a TNSS≤2 prior to the screening NAC (time 0), and peak TNSS≥7 within one hour of NAC initiation.

Eligible subjects were randomized to receive study drug or placebo on study day 1 (14 days after screening visit 2). On study days 8, 29, 57, and 85, subjects underwent NAC using the same allergen titration required for each individual subject to reach TNSS≥7 at their 2^{nd} screening visit, not to exceed the maximum dose established in the screening visit, regardless of whether TNSS≥7 was reached. At each study visit, TNSS, as well as VAS nasal symptoms score (0 to 100 scale) and peak nasal inspiratory flow (PNIF, measured in nasal patency, l/min) were measured, were measured pre-NAC, then at 10, 30, and 60 min during the first hour, and once per hour for 8 hours to measure the late phase response (LPR). Serum samples were collected at each study visit, and a repeat cat-SPT was performed on study days 29 and 85.

### Results

### REGN1908-1909 Blocked the Early Phase Allergic Response to Nasal Allergen Challenge in Cat-Allergic Patients.

A Nasal Allergen Challenge (NAC) with cat hair extract was selected for measurement of allergy symptoms. Intranasal instillation of allergen causes local allergic symptoms, such as nasal congestion, itching, sneezing and rhinorrhea, which are associated with IgE-mediated mast cell and basophil degranulation and peak within the EPR (Durham, M. D., et al., (2016), Journal of Allergy and Clinical Immunology 138 (4). Elsevier Inc.: 1-12; Scadding, G., et al., (2015), Clinical and Experimental Allergy: Journal of the British Society for Allergy and Clinical Immunology 45 (3): 613-23). Therefore, the primary efficacy analysis was change in Total Nasal Symptom Score (TNSS) area under the curve (AUC) over the first hour after a NAC [TNSS AUC(0-1h)] from pretreatment to day 8 challenge.

The change in clinical symptoms [TNSS AUC(0-1h)] from baseline to day 8 NAC was significantly reduced in patients receiving a single SC dose of REGN1908-1909 (600 mg) compared to placebo. REGN1908-1909 treatment led to sustained and clinically meaningful improvement in symptoms (commonly defined as >20% reduction in total symptom score compared to placebo):

REGN1908-1909 treatment also reduced the hypersensitivity response to cat hair extract delivered to the skin in a titrated skin prick test (cat-SPT).

Thus, a single SC dose of REGN1908-1909 in cat-allergic subjects resulted in a clinically meaningful and sustained reduction in total nasal symptoms by blocking the early allergic response to nasal challenge with cat allergen.

### Example 5. A Phase 2, Randomized, Double-Blind, Placebo-Controlled, Parallel-Group Study in Cat-Allergic Patients with Asthma to Evaluate the Efficacy of a Single Dose of REGN1908-1909 to Reduce Bronchoconstriction Upon Cat Allergen Challenge

A phase 2 clinical study (NCT03838731, incorporated herein in its entirety) was carried out to investigate the prophylactic effect of REGN1908-1909 (anti-Fel d1) to reduce acute bronchoconstriction in cat-allergic patients with mild asthma when exposed to cat allergen in a controlled environmental exposure unit (EEU). Environmental exposure units are enclosed spaces that control temperature, air flow, and humidity, and provide spatial distribution and temporal stability of allergen concentration that simulates natural circumstances.

This 2-arm, placebo-controlled, double-blind, single-dose, randomized, parallel-group proof-of-concept (POC) study was planned to enroll approximately 60 cat-allergic patients with allergic rhinitis (AR), not currently living with a cat who have a history of mild asthma defined by the Global Initiative for Asthma stage 1 (GINA stage 1) as per the 2008 guidance as follows: asthma symptoms are controlled with short-acting beta agonists as needed, asthma symptoms are rare, there is no night awakening due to asthma, no exacerbations in the last year, and normal FEV1 (FEV180% predicted value). Patients were randomized (1:1 ratio) to receive a single dose of REGN1908-1909 or placebo subcutaneously. The primary endpoint of the study was to evaluate the time to acute bronchoconstriction after allergen exposure (referred to as the early asthmatic response or EAR).

The instant phase 2, randomized, double-blind, parallel-group, single-dose study in approximately 60 cat-allergic patients with mild asthma (GINA stage 1) with rhinitis, with or without conjunctivitis to cat hair, who are not living with cats, was carried out in order to evaluate the efficacy of a prophylactic, single 600 mg subcutaneous (SC) dose of REGN1908-1909 to prevent acute, allergic, lower respiratory symptoms during exposure to cat allergen as measured by spirometry. This single-site study incorporated clinical monitoring appropriate for conducting a study measuring a mild-to-moderate reduction in FEV1 in the asthmatic population.

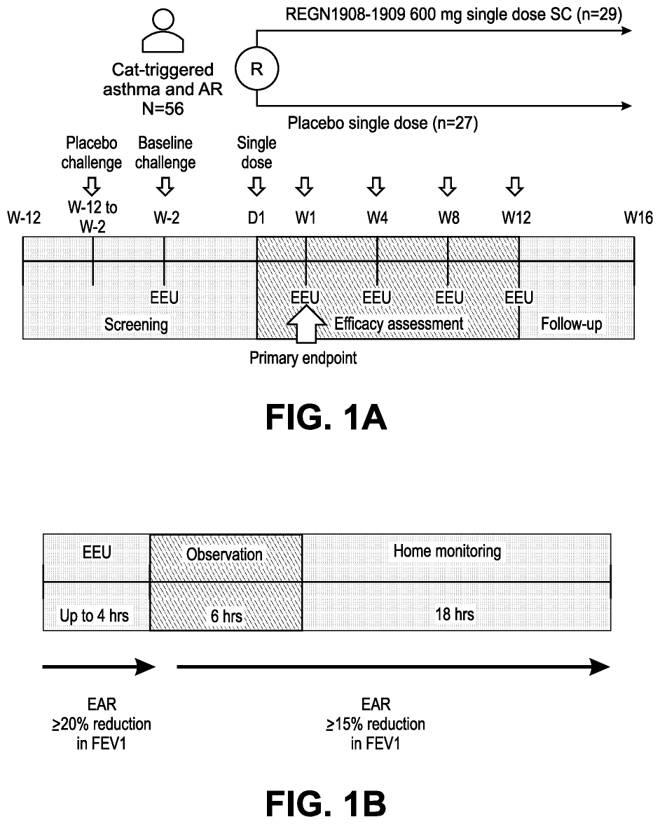

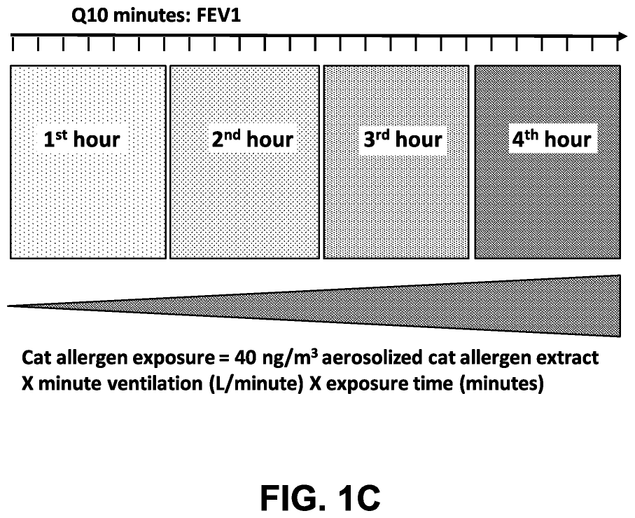

The study consisted of up to a 12-week screening period followed by 1:1 randomization and treatment of eligible patients with double-blind, single SC dose of study drug at day 1 followed by a 12-week assessment period and then a 4-week safety follow-up period. Patients underwent a saline challenge (placebo challenge) during screening day −85 to −14, where they were exposed to nebulized normal saline for 2 hours in the EEU, while FEV1 was monitored every 10 minutes. Patients then underwent an allergen challenge (involving exposure to cat allergen in the EEU) during screening (baseline challenge) for up to 2 hours, and then allergen challenges on study drug (challenge) at days 8 (week 1), 29 (week 4), 57 (week 8), and 85 (week 12) for up to 4 hours (FIG. 1). Although the amount of cat allergen that is nebulized into the EEU is continuously 40 ng/m³ Fel d1 (approximately), the individual exposure increases over time in association with minute ventilation (the volume of air that can be inhaled or exhaled for 1 minute). Therefore, minute ventilation was measured for; each patient at baseline using spirometry, so that the cat allergen airborne exposure, as a product of the individual's minute ventilation and time, could be assessed at each Cat Allergen Challenge on days 8, 29, 57, and 85. A final safety follow-up visit at week 16, 4 weeks after the last Cat Allergen Challenge, will be performed to assess for asthma symptoms after undergoing repeated Cat Allergen Challenges.

### Patient Population

The patients were adults 18-65 years inclusive of males and females with cat-induced asthma (GINA 1) and AR with or without conjunctivitis symptoms with cat sensitization confirmed at screening.

Patients had to meet the following criteria to be eligible for inclusion in the study described herein: i) generally healthy men and women between the ages of 18 and 65 inclusive at the time of screening; ii) documented or patient reported history (for at least 2 years) of symptomatic cat hair-triggered asthma with rhinitis with or without conjunctivitis as defined by all of the following criteria: aa) positive skin prick test (SPT) with cat hair extract (mean wheal diameter at least 5 mm greater than a negative control) at screening; bb) positive allergen-specific IgE (sIgE) tests for cat hair and Fel d1 (>0.35 kAU/I at screening); cc) history of asthma GINA 1; dd) screening FEV1 ≥70 predicted after withholding long-acting β2-agonists for >36 hours and short-acting β2-agonists for >6 hours; and ee) demonstrated ≥20 fall in FEV1 within 2 hours during Cat Allergen Challenge in EEU and ability to withstand exposure for at least 10 minutes during screening; iii) willing and able to comply with clinic visits and study-related procedures; iv) provide informed consent signed by study patient or legally acceptable representative; v) patients covered by health social identification number; vi) able to understand and complete study-related questionnaires; vii) no cat exposure at home for the past year and must continue having no exposure at home during the study; cat exposure outside of the home shall be avoided for at least 1 week prior to any Cat Allergen Challenge and during the defined follow-up period; and vii) less than 10 pack-years of smoking history.

Patients meeting any of the following criteria were excluded from the study described herein: i) patients who experience a ≥10 fall in FEV1 at 3 consecutive spirometry measurements during the placebo challenge; ii) positive human immunodeficiency virus (HIV) test; iii) positive hepatitis test (HBsAg and hepatitis C antibody); iv) history of significant multiple and/or severe allergies (including latex gloves) or has had an anaphylactic reaction or significant intolerability to prescription or nonprescription drugs or food; v) participation in a prior REGN1908-1909 clinical trial; vi) history of severe anaphylactic or severe asthmatic reactions to cat exposure; vii) active lung disease other than asthma; viii) treatment with an investigational drug within 2 months or within 5 half-lives (if known), whichever is longer, prior to screening; ix) persistent chronic or recurring acute infection requiring treatment with antibiotics, antivirals, or antifungals, or any untreated respiratory infections within 4 weeks prior to screening; x) serum creatinine, creatinine phosphokinase (CPK), alkaline phosphatase, hepatic enzymes (aspartate aminotransferase [AST] and alanine aminotransferase [ALT]), total bilirubin (unless the Investigator has evidence that increased indirect bilirubin corresponds to a Gilbert's-type syndrome) that exceed 1.5× the upper limit of normal (1.5×ULN), or any laboratory findings showing evidence of organ dysfunction or any clinically significant deviation from the normal range, as decided by the Investigator at the screening visit; xi) any medical illness, which in the judgment of the investigator could preclude participation in the study or in whom treatment with epinephrine or beta-2 agonists or systemic corticosteroids would pose an increased risk (e.g., history of cardiovascular disease, hypertension, diabetes, etc.); xii) patients taking any prohibited treatment (within time period before screening or EEU visit), including topical or systemic first generation H1 antihistamines (5 days), topical or systemic second generation H1 blockers (7 days), systemic anti-H2 (8 days), Astemizole (6 weeks), cromoglycates, leukotriene modifiers (7 days), systemic steroid treatment (8 weeks prior to screening and during the study, but allowed if clinically indicated for a LAR treatment after exposure to cat allergen in the EEU), topical steroids (48 hours), short-acting β2 agonists (8 hours), long-acting β2 agonists (e.g., salmeterol) (36 hours), ultra-long-acting β2 agonists (e.g., indacaterol, vilanterol, olodaterol) (48 hours), anticholinergics e.g., Ipratropium (Atrovent 40 pg) (12 hours), long-acting anti-muscarinic agents (7 days), methylxanthynes (e.g., oral theophylline) (24 hours), intramuscular corticosteroids (3 months prior to screening and during the study), systemic or topical calcineurin inhibitors (14 days prior to screening and during the study), tricyclic antidepressants/antipsychotics (14 days), topical decongestants (72 hours), and/or caffeine-containing drinks or products (8 hours of EEU visits only), as well as history of SIT with cat allergen or vaccines against cat allergy within 5 years of screening, SIT with any allergen within 6 months of screening, beta-blockers and ACE inhibitors during the study period, immunomodulatory therapy, anti-IgE, or other biological, agent-based antagonist therapy in the 6 months prior to baseline (e.g., cyclosporine) or during the study, change in prescription medications within 4 weeks before screening, aspirin and any nonsteroidal anti-inflammatory drug, and/or active treatment for respiratory infections (antiviral, antifungals, or antibiotics) within 4 weeks prior to screening or EEU; xiii) use of systemic corticosteroids within 8 weeks prior to screening visit 1; xiv) use of anti-IgE or other biological therapy within 6 months prior to screening visit 1; xv) history of SIT with cat allergen or vaccines against cat allergy within 5 years of screening visit 1; xvi) SIT with any allergen within 6 months prior to screening visit 1; xvii) significant rhinitis, or sinusitis, due to daily contact with other allergens causing symptoms that are expected to coincide with the baseline or the final cat allergen exposure unit assessments as assessed by the investigator, before each exposure; xviii) patients who anticipate major changes in allergen exposure in their home or work environments that are expected to coincide with the baseline or the final cat allergen exposure assessments as assessed by the investigator; xix) hospitalization for any reason within 30 days prior to screening visit 1; xx) history of life-threatening asthma, defined as an asthma episode that required intubation and/or was associated with hypercapnia, respiratory arrest, and/or hypoxic seizures; xxi) treatment of asthma requiring systemic (oral or parenteral) corticosteroid treatment more than twice within 12 months or once within 3 months prior to screening or has been hospitalized or has attended the ER/Urgent Care facility for asthma more than twice in prior 12 months before screening; xxii) history of hypersensitivity to corticosteroids or antihistamines, or drug treatment excipient; xxiii) known sensitivity to doxycycline, tetracyclines, or to any of the components of the investigational product formulation; xxiv) positive serum human chorionic gonadotropin pregnancy test at the screening visit or urine pregnancy test at the baseline visit; xxv) pregnant or breastfeeding women; xxvi) women of childbearing potential who are unwilling to practice highly effective contraception prior to the initial dose/start of the first treatment, during the study, and for at least 6 months after the last dose of study drug; xxvii) sexually active men who are unwilling to use certain medically acceptable birth control during the study drug treatment period and for 6 months after the last dose of study drug; xxviii) inability to understand and act upon the information provided (what to do in an emergency situation, patient has difficulty understanding/communicating, etc.); xxix) patient under legal custody, guardianship, or curatorship; and xxx) screening asthma control test (ACT) <20 at any screening visits.

### Patient Screening and Eligibility

Patients were assessed for eligibility during a 3-part screening period. During screening visit 1, patients underwent eligibility criteria evaluation, collection of medical history, physical examination, vital signs, cat hair extract SPT, standard regional SPT (Dermatophagoides pteronyssinus, Dermatophagoides farinae, dog, aspergillus, Alternaria, birch pollen, 3 grasses pollen, ash pollen), electrocardiogram (ECG), and spirometry. Blood samples were collected for anti-cat hair and Fel d1-specific anti-IgE along with other common allergen determination and serum laboratory testing.

Eligible patients underwent a screening placebo challenge in the EEU for up to 2 hours. Patients who experienced a ≥10 fall in FEV1 at 3 consecutive spirometry measurements during this placebo challenge were excluded (e.g., over 3 separate consecutive spirometry readings during the 2 hours EEU patients must demonstrate 0% fall in FEV1). Baseline skin prick testing with serial allergen titration with cat allergen was performed at this visit in patients who successfully completed the placebo challenge without experiencing a ≥10 fall in FEV1 at 3 consecutive spirometry measurements.

If patients were eligible after the placebo challenge in the EEU, they underwent a Controlled Cat Allergen Challenge for a maximum of 2 hours. Patients had to demonstrate ≥20 fall in FEV1 during exposure and ability to withstand exposure for at least 10 minutes, or they were excluded. Patients were removed from the EEU, once they demonstrated ≥20 fall in FEV1, and the time to this reduction in minutes was recorded. The cat EEU visit 3 during screening had to be at least 3 weeks from the cat EEU visit 6 (visits must be spaced apart by at least 3 weeks to avoid allergen priming).

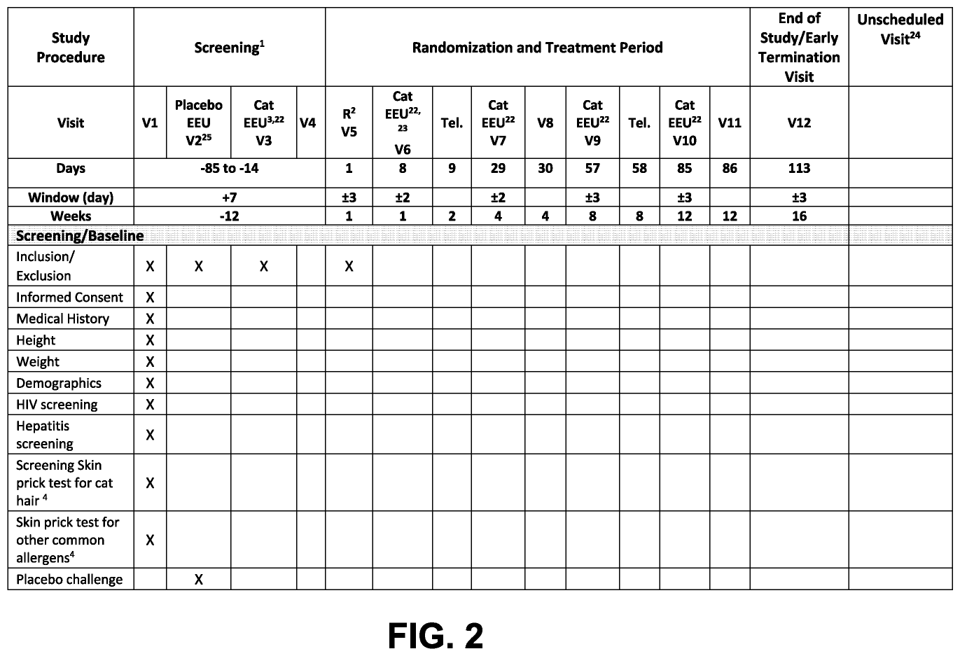

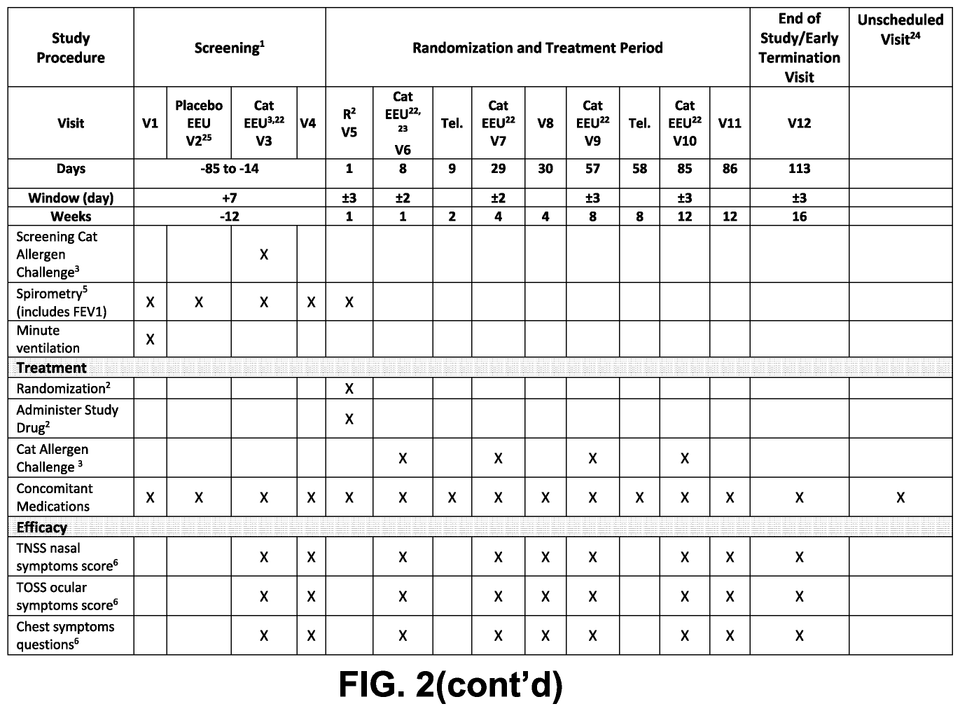

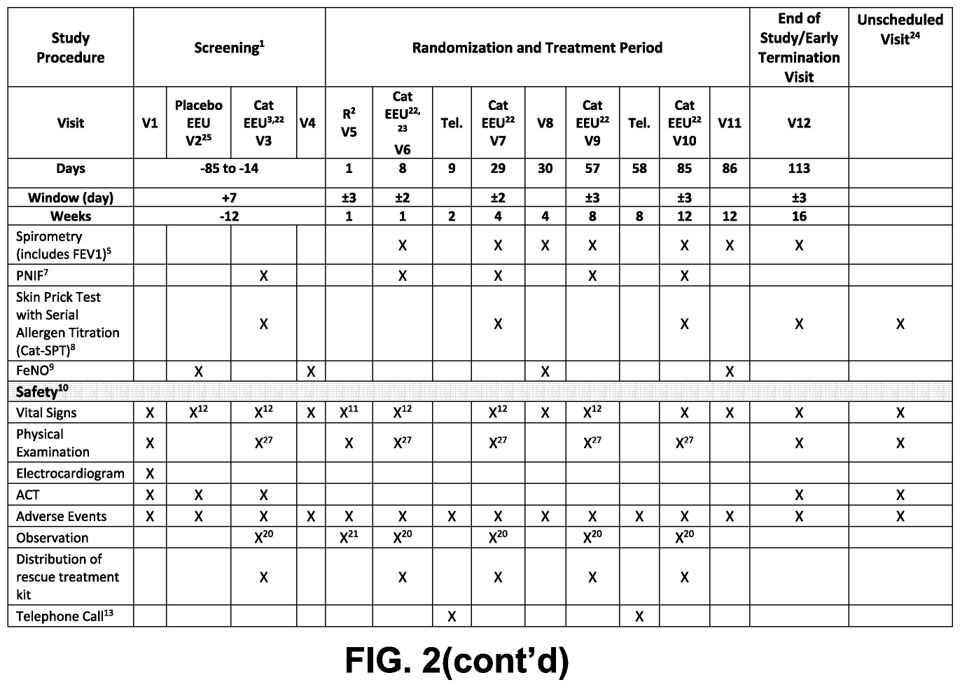

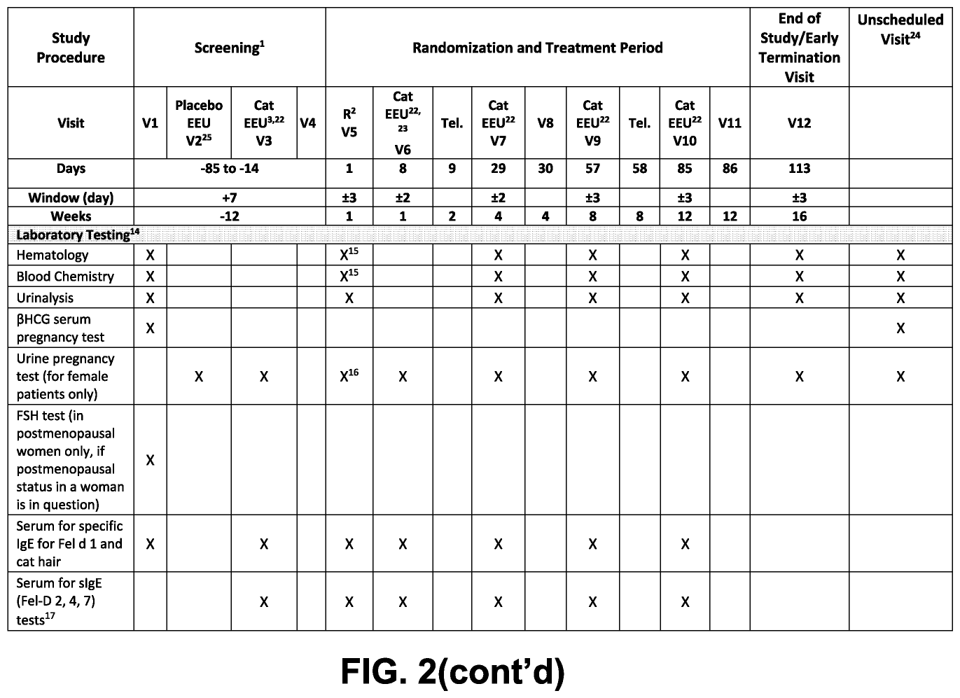

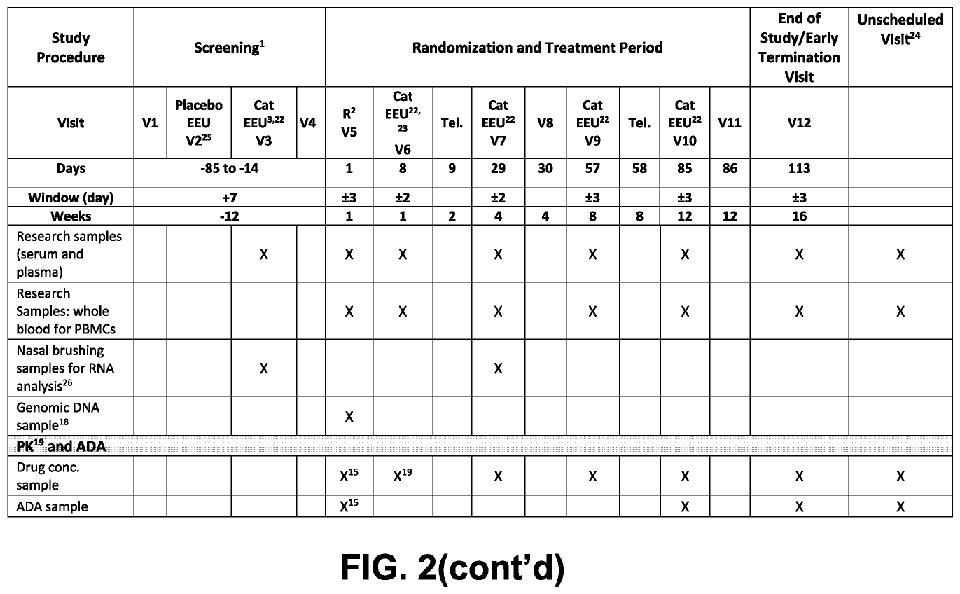

A full schedule of events is shown in FIG. 2. Footnotes:
1. All screening-related procedure should be performed between day −85 to −14
2. Randomization/study drug administration (visit 5) must occur within 14 to 28 days of visit 3, so that the Cat Allergen Challenge in the EEU at visit 3 and visit 7 are separated by at least 21 days, and that randomization occurs within 28 days of visit 3.
3. Controlled Cat Allergen Challenge is performed as described in the study manual.
4. Screening Skin prick test for cat hair and skin prick testing for other common allergens is performed as described in the study manual. If screening skin prick test for cat hair is negative at screening visit 1 (mean wheal diameter less than at least 5 mm greater than a negative control), then other screening visit 1 procedures do not need to be performed as the patient will have failed screening based upon cat hair skin prick testing (e.g., perform skin prick testing prior to other allergen skin prick testing, blood draw, spirometry, etc.).
5. At screening visit 1, a spirometry session will be completed to check for FEV1 inclusion criteria. At visit 2, spirometry will be performed at baseline prior to the placebo EEU challenge, and then every 10 minutes during the placebo challenge. On the Cat Allergen Challenge visits (3, 6, 7, 9 & 10) spirometry will be performed at baseline prior to entry into the EEU, every 10 minutes during the EEU exposure, every 30 minutes in the observation room for 6 hours after leaving the EEU, and then every hour for 18 hours after EEU exposure, except during sleeping. At visit 5, on the day of randomization a spirometry session will be performed twice: once prior to receiving the study drug and once prior to leaving the clinical trial unit. Spirometry may also be performed additional times as needed.
6. TNSS, TOSS, and chest symptom questions are assessed prior to Cat Allergen Challenge, approximately every 20 minutes during the challenge in the exposure unit, every 1 hour for 6 hours post-challenge while patients are being observed in the observation room, and then every 2 hours up to 18 hours after EEU exposure, while they are home, except for the time that they are sleeping. Further details are provided in the study manual.
7. PNIF is assessed prior to Cat Allergen Challenge, approximately at the time of the EAR, and approximately 6 hours post-challenge while patients are being observed in the observation room. Further details are provided in the study manual.
8. Titrated SPT with Serial Allergen Titration with cat allergen will be performed at screening visit 3, days 29, 85, and 113. On days when an allergen challenge is performed, the test will be performed prior to allergen challenge.
9. FeNO is assessed during screening and then 24 hours after EEU exposure at day 30 and day 86.
10. All safety assessments performed at screening have to be normal and checked against the inclusion/exclusion criteria prior to REGN1908-1909 administration on day 1 (baseline).
11. On day 1, vital signs are taken prior to PK draw, prior to REGN1908-1909 administration, and at 2 hours (±10 min) after completion of the injection.
12. Vital signs are taken prior to entry into the EEU, at exit from the EEU, and prior to leaving the clinical trial unit, and any additional times as needed.
13. Telephone call to collect AEs and concomitant medications, including any medications used from the rescue treatment kit, up to 24 hours after the EEU exposure.
14. Total blood draw at any visit will never exceed 60 mL. Blood volumes are never to exceed 350 mL in 12 weeks.
15. Samples are collected prior to administration of REGN1908-1909.
16. On day 1, urine pregnancy test is completed prior to administration of REGN1908-1909 in women of childbearing potential. Post-menopausal women do not need pregnancy testing.
17. Total IgE and allergen-specific serum IgE levels (Fel d 2, Fel d 4, Fel d 7).
18. Genomic analysis is mandatory for all patients enrolling in the study. One DNA sample is to be collected on day 1/randomization, but if this sample collection was omitted at baseline, it can be collected at any subsequent visit. DNA samples may be stored for up to 10 years for future research use.
19. PK samples are drawn at any time in the outpatient visit day from day 8 through day 113 (end of study) visit.
20. After exiting the EEU, patients are monitored outside the EEU in an observation room for approximately 6 hours.
21. Patients will be observed for 6 hours in the observation room after receiving a single dose of REGN1908-1909 or placebo.
22. Blood draws should be performed before the EEU exposure
23. Cat EEU visit 3 and visit 6 must be spaced at least 3 weeks apart
24. Assessments and procedures at the unscheduled visit(s) are to be performed at the discretion of the principle investigator.
25. Screening visits 1 and 2 may be combined into one visit if the subject has a historical, positive cat SPT or cat IgE that was completed in the last 12 months.
26. Nasal brushing samples will be collected from the patients before and after Cat Allergen Challenge in the EEU on the day of the screening challenge (Visit 3) and day 29 (Visit 7) Before the challenge in the EEU, a baseline nasal brushing will be performed in 1 nare and the samples will be processed. Six hours from the start of the EEU challenge, a nasal brushing will be performed in the contralateral nare.
27. Physical exam will be performed prior to entering the EEU and approximately 6 hrs after the exit or before leaving the observation room, whichever is later.

### Randomized Single-Dose Treatment/Follow-Up Period

Approximately 60 adult patients who met the eligibility criteria were to be randomized in a 1:1 ratio into 1 of 2 treatment regimens (n=30 in each group):
REGN1908-1909 600 mg subcutaneous (SC) dose at day 1; and
Matching placebo SC dose at day 1.

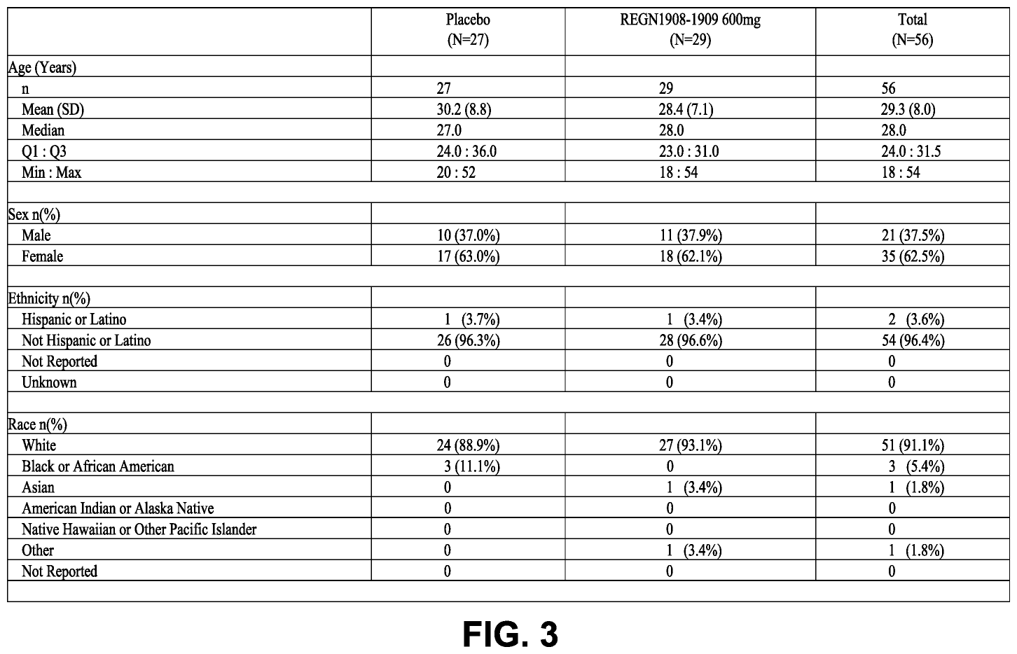

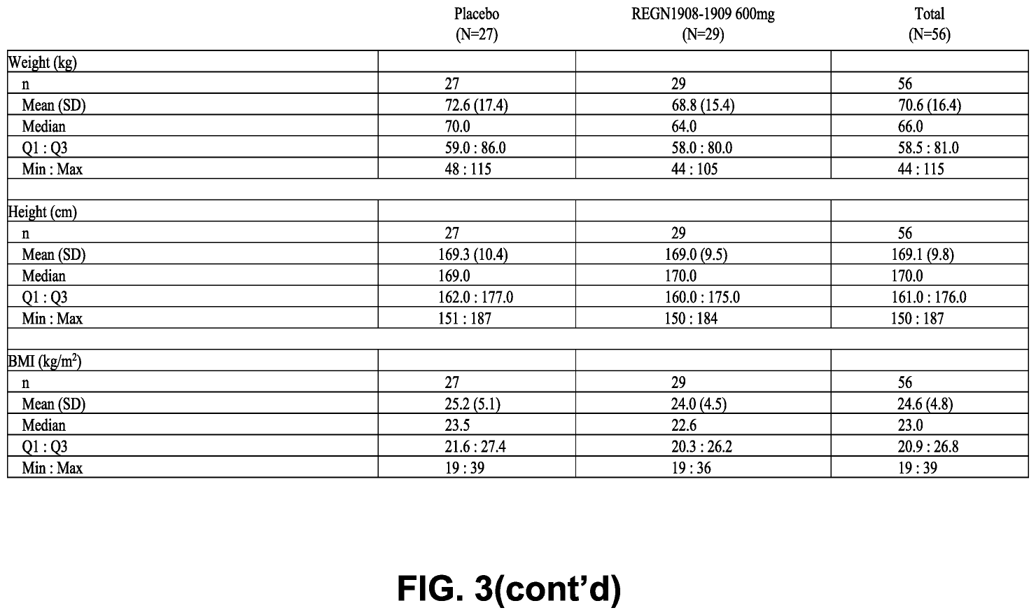

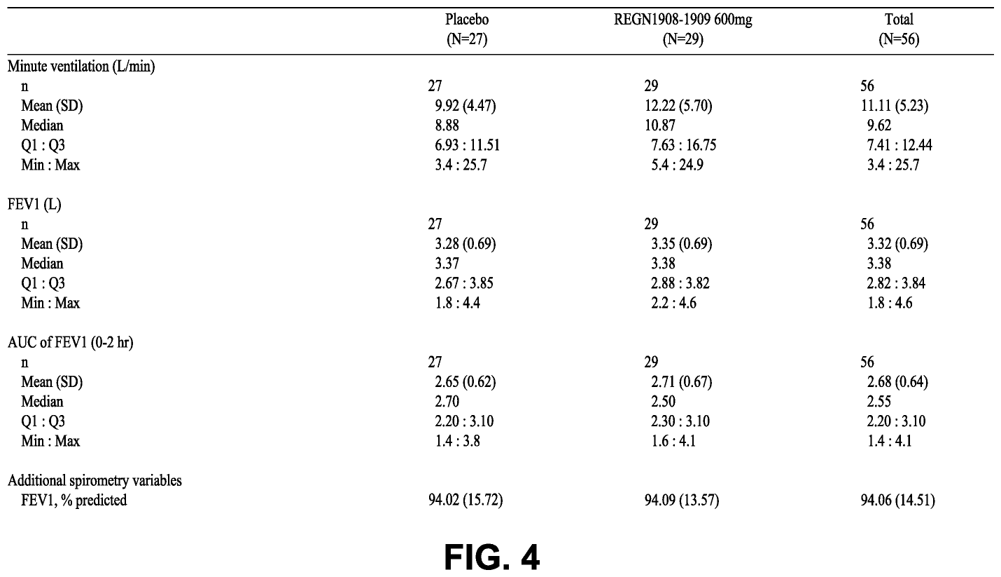

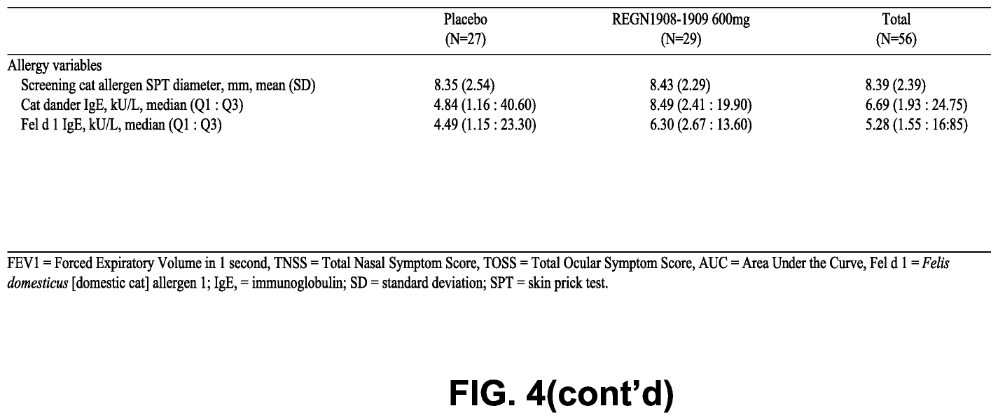

In fact, a total of 56 patients (cat-allergic, with mild asthma) were randomized, 27 to placebo and 29 to REGN1908-1909. All patients randomized received their intended study treatments. A total of 54 (96.4%) patients completed the study (26 receiving placebo and 28 receiving REGN1908-1909). Baseline demographics are summarized in FIG. 3, and baseline characteristics are summarized in FIG. 4.

Pre-lyophilized REGN1908 and REGN1909 were each formulated in a buffered, aqueous solution at pH 5.8. REGN1908 and REGN1909 drug product were supplied separately as lyophilized cakes in 20 mL glass vials.

For SC administration, these drug products were each reconstituted with 2.3 mL of sterile water for injection, yielding a final concentration of 100 mg/mL REGN1908 or 100 mg/mL REGN1909 in histidine, polysorbate 80, and sucrose. A total volume of 2.0 mL of the reconstituted liquid could be withdrawn from the glass vial. The reconstituted liquids were mixed as a 1:1 cocktail. The resulting mixture had a concentration of 100 mg/mL total of REGN1908 1909 (50 mg/mL REGN1908 and 50 mg/mL REGN1909). A 2.0 mL injection of the cocktail provided a total dose of 200 mg REGN1908-1909.

Placebo was supplied in vials that matched, but did not contain the protein. Colored, transparent “blinding labels” were placed on drug product syringes to blind staff that administered drug products.

For the instant single-dose study, study drug was administered SC on day 1, by the investigator, or other qualified study personnel. Patients were randomized in a 1:1 ratio to receive 600 mg REGN1908-1909 or placebo.

Three 2.0 mL SC injections were administered in the abdomen in different quadrants.

All patients were assessed at scheduled visits for safety, laboratory, and clinical assessments for 16 weeks after the single SC dose of study drug treatment. The duration of the 16-week follow-up period (12-week assessment period and then a 4-week safety follow up) was based on the time expected for drug levels to be insignificant after the single 600 mg SC dose of REGN1908-1909.

### Controlled Cat Allergen Study

After randomization, patients underwent serial Controlled Cat Allergen Challenges (via cat allergen exposure in the environmental exposure unit (EEU)) for a maximum of 4 hours at days 8, 29, 57, and 85 for evaluation of study drug efficacy. Before entry into the cat allergen exposure unit, prohibited medications were withheld, and patients received a physical examination; chest, nasal, and ocular symptoms, as well as pulmonary function, were evaluated and recorded. The challenge did not proceed, if patients had a TNSS greater than 4, an asthma control test (ACT) score <20 at any of the screening visits, or FEV1 of less than 70% of predicted value. Patients could leave the exposure unit at any time and wore a disposable suit to protect them from allergen inside the EEU and to avoid contaminating the EEU with other allergens brought in from their clothing, which could interfere with the cat allergen studied.

The EEU was operated under conditions to maintain stringent control of temperature, relative humidity, ventilation rate, particle number, particle size, and concentration of airborne cat hair extract. Standardized allergen extracts were administered through a nebulizer (SinapTec®) to ensure uniform particle count and size in the EEU. Ten particle counters (APEX R05) positioned in the EEU provided continuous monitoring to confirm uniformity of patient allergen exposure during the allergen challenge. Airborne Fel d1 concentrations were sampled at 5 locations in the EEU using 25 mm round fiber filters (Millipore Corp, Bedford, MA) and a multiplex proteomics platform to measure airborne concentrations of cat allergens. Fel d1 levels was measured with a Fel d1-specific ELISA (King, et al., 2013 J Immunological Methods 387: 89-95). Fel d1 levels will be measured with a Fel d1-specific ELISA performed by Indoor Biotechnologies.

As depicted in the study flow diagram of FIG. 1B, at baseline, patients were exposed in the EEU for up to 2 hours, until they demonstrated an early phase response (20% fall in FEV1), and then patients were observed outside of the EEU for 6 hours to observe if/when a late phase response would occur (15% fall in FEV1). At subsequent challenge visits (days 8, 29, 57, and 85), patients were exposed in the EEU for up to 4 hours, until they demonstrated an early phase response (20% fall in FEV1), or until they departed due to clinically significant allergic and/or asthma symptoms, and then patients were observed outside of the EEU for 6 hours to observe a late phase response (15% fall in FEV1). Although the amount of Fel d1 aerosolized into the EEU was maintained at 40 ng/m³, the individual exposure actually increased over time in association with minute ventilation (the volume of air that can be inhaled or exhaled during 1 minute) (FIG. 1C).

### Assessments

### Spirometry/FEV1

Study staff-supervised spirometry was performed in all patients at visits shown in FIG. 2 with a standard measurement of FEV1. FEV1 was measured by means of spirometry at 10 minute intervals during placebo and cat allergen exposures in the EEU, every 30 minutes during the 6 hour observation period after leaving the EEU, and hourly using at-home spirometry after leaving the clinical trial unit up to 18 hours after exposure, excluding hours when the patient is sleeping. Spirometry could also be performed additional times as needed. For example, additional spirometry was performed when prompted by asthma symptoms and/or at the discretion of the investigator. In the case where a patient had multiple spirometry efforts from the same time point, the study staff selected an effort for use in analysis based on the quality of the efforts as assessed by a flow volume loop.

Spirometry was measured on the day of randomization prior to receiving the study drug and was measured after 6 hours prior to leaving the clinic. At the end of the 6-hour monitoring period, patients underwent a physical exam and vital signs, including spirometry, and were discharged home, if there were no abnormal findings, and if FEV1 was ≥90 of baseline. Otherwise, if FEV1 was <90% of the baseline value, patients continued to be monitored at the clinic until they met criteria for discharge.

Spirometry measurements included FVC (L), FEV1 (L), FEV1/FVC (%), PEF (L/s), FEF 25-75 (L/s). Minute ventilation (L/min) was also be measured using spirometry at screening 1 time while the patient was at rest, and this value was used to calculate allergen exposure throughout the study (minute ventilation×Fel d1 40 ng/m³×time).

### Asthma Control Test

The asthma control test (ACT), which uses a 5-point Likert scale, was used prior to the screening cat allergen EEU to determine whether the patient's asthma was well controlled, and again at the final safety follow-up study visit. Individual respiratory symptoms of chest tightness/shortness of breath/trouble breathing, wheezing, and coughing are evaluated on a 4-point Likert scale ranging from 0 (none) to 3 (severe).

### Patient-Reported Allergic Symptoms

Patient-reported outcomes (PROs) are essential to evaluate symptoms, impact of symptoms on activities of daily living, and treatment response. Patient-reported allergic symptoms (nasal, ocular, and chest symptoms) were recorded by patients using a 4-point Likert scale. Total nasal symptom score (TNSS) is from 0 to 12 and is based on assessment of 4 nasal symptoms graded on a Likert scale ranging from 0 (none) to 3 (severe) for congestion, itching, and rhinorrhea, and from 0 (none) to 3 (5 or more sneezes) for sneezing. Total ocular symptom score is from 0 to 12 and is based on a 4-point Likert scale ranging from 0 (none) to 3 (severe) for itching/burning, redness, swelling/puffiness, and tearing/watery eyes. Individual respiratory symptoms of chest tightness/shortness of breath/trouble breathing, wheezing, and coughing are evaluated on a 4-point Likert scale ranging from 0 (none) to 3 (severe).

### Fractional Exhaled Nitric Oxide (FeNO)

Measurement of FeNO, a marker of airway inflammation, was analyzed from exhaled breath condensates obtained at baseline and 24 hours after the Controlled Cat Allergen Challenges during screening, on day 30 and day 87. Fractional exhaled nitric oxide (measured in parts per billion, which is equivalent to nanoliters per liter) was assessed at each time point according to FIG. 2. FeNO is sampled by the nitric oxide analyzer, and the resultant nitric oxide profile versus time or exhaled volume, together with other exhalation variables (e.g., airway flow rate and/or pressure), is captured and displayed.

### Peak Nasal Inspiratory Flow (PNIF)

Peak nasal inspiratory flow (PNIF) (measured in nasal patency, L/min) was assessed at each time point according to FIG. 2. PNIF was measured and recorded approximately at the time of the EAR during the Cat Allergen Challenge in the EEU and then approximately 6 hours after the EAR during the observational period.

### Screening Skin Prick Test (SPT)

Standard SPT with cat hair extract and other common allergens was only performed at screening visit 1 to assess sensitization status.

SPT with Serial Allergen Titration with Cat Hair Extract

Skin Prick Test with Serial Allergen Titration with cat hair extract (Cat-SPT) was performed at screening visit 3 and days 29 and 85 and end of study to confirm pharmacodynamic effects of REGN1908-1909 on wheal size response (mediated by mast cell degranulation). Serial dilutions of cat hair extract were placed in duplicate, using skin prick tests on the patient's forearm, and mean wheal diameters were measured 15 minutes after placement.

### Asthma Control Test

The asthma control test (ACT) is comprised of patient-reported asthma symptoms using a 5 point Likert scale comprised of 5 elements rated 1-5, with higher scores representing better asthma control. ACT was performed at screening and at the end of the study at time points according to FIG. 2.

### Serum Antibodies

Serum allergen-specific IgE levels (cat hair, Fel d1) were measured at screening visit 1 and as described in FIG. 2. Serum allergen-specific IgE levels (Fel d 2, Fel d 4, Fel d 7 and other common allergens) were measured at baseline and days of EEU challenge to assess sensitization status and to evaluate the relationship between response to REGN1908-1909 and poly/mon-sensitization. Additional subclasses of serum antibodies (e.g., IgG4) against Fel d1, d 2, d 4, d 7 could also be measured.

### Nasal Brushing Samples

Nasal brushing samples were collected from the patients before and after Cat Allergen Challenge in the EEU on the day of the screening challenge (Visit 3) and day 29 (Visit 7). Before the challenge in the EEU, a baseline nasal brushing was performed in 1 nare, and the samples were processed. Six hours from the start of the EEU challenge, a nasal brushing was performed in the contralateral nare, and the sample was processed. Nasal brushing was performed by a clinician experienced in nasal procedures under direct visualization, by inserting a soft, sterile cytobrush into the nare alongside the inferior nasal turbinate of 1 nostril approximately 0.5 cm above the floor of the nose and 1.5 cm into the nasal cavity and rotating the brush 180 degrees once to the lateral aspect of the nostril. RNA was extracted from nasal brushing samples and used to perform RNA sequencing to determine changes in type 2 inflammation in the nasal mucosa.

Interference Assay with REGN1908-1909

Serum samples from patients before drug exposure were tested in an in vitro competition assay to assess whether REGN1908-1909 could inhibit the binding of endogenous anti-fel-d1 IgE to the allergen.

### Efficacy Analyses

### Primary Efficacy Analysis

The primary endpoint in the study was the time to EAR upon Controlled Cat Allergen Challenge in an EEU on day 8. The time to EAR was defined as the time to a ≥20% reduction in FEV1 (forced expiratory volume over one second) from baseline, or when the patient voluntarily departed the EEU due to clinically significant allergic and/or asthma symptoms and/or were treated with rescue medications. The time to EAR for each treatment was examined using Kaplan Meier estimates with patients being censored at 4 hours, if they did not experience an EAR and remained in the EEU for 4 hours. If a patient left the EEU before experiencing an EAR and for reasons unrelated to their clinical symptoms, they were censored at the time of EEU departure. The median time to EAR for each treatment group and the corresponding 95% confidence intervals were presented for each Controlled Cat Allergen Challenge. A formal comparison of time to EAR in the treatment groups at day 8 were performed using a Cox proportional hazards model, adjusting for allergen exposure and time to EAR in the baseline Cat Allergen Challenge. The ratio of Fel d1 IgE to Cat Hair IgE at baseline was also explored as potential covariate in the model. In patients who received EEU-related medications (including medications given on-site and rescue medications taken at-home), the impact of the carryover of medications between challenges was explored. A one-sided statistical test of the hazard ratio for placebo compared to drug was performed.

### Secondary Efficacy Analysis

The secondary endpoints included one or more of:
time to EAR upon Controlled Cat Allergen Challenge in an EEU on days 29, 57, and 85;
change and percent change in AUC of the FEV1 during a Controlled Cat Allergen Challenge over the exposure interval (maximum of 4 hours) from the baseline Controlled Cat Allergen Challenge to challenges on days 8, 29, 57, and 85;
change in AUC of patient-assessed nasal symptoms induced by a Controlled Cat Allergen Challenge over the exposure interval (maximum of 4 hours) from the baseline Controlled Cat Allergen Challenge to challenges on days 8, 29, 57, and 85;
change in AUC of patient-assessed ocular symptoms induced by a Controlled Cat Allergen Challenge over the exposure interval (maximum of 4 hours) from the baseline Controlled Cat Allergen Challenge to challenges on days 8, 29, 57, and 85;
change and percent change in cat allergen quantity as experienced by patients during exposure (measured by Fel d1 allergen concentration (ng/m³)×minute ventilation×time in hours) from the baseline Controlled Cat Allergen Challenge to challenges on days 8, 29, 57, and 85; and/or
incidence rates of treatment-emergent adverse events (TEAEs) and serious TEAEs through end of study.

The time to EAR for Cat Allergen Challenges on days 29, 57, and 85 was evaluated in a similar way to the primary analysis. The time course of asthma response comprising EAR and LAR was visualized and summarized.

The AUC of percentage change from baseline FEV1 during the Controlled Cat Allergen Challenge was analyzed by mixed-effect model repeated measures (MMRM) with the treatment, time, treatment-by-time interaction, and day of challenge as factors and baseline FEV1 as a continuous covariate. An unstructured covariance structure was utilized; if the model did not converge, an autoregressive structure was employed. Between-group estimates and nominal p values were reported for Controlled Cat Allergen Challenges on days 8, 29, 57, and 85. The AUC was a time-adjusted-based duration of exposure to account for patients exiting the EEU at different times. Missing data from Controlled Cat Allergen Challenges on days 8, 29, 57, or 85 were accounted for via MMRM. The AUC of percent change in TNSS and TOSS and change and percent change in cat allergen exposure from the baseline Cat Allergen Challenge were analyzed in a similar manner.

The change and percent change in cat allergen quantity (tolerated exposure) from the baseline Cat Allergen Challenge, was also analyzed using a similar MMRM model with the treatment, visit, and treatment by-visit interaction as factors and the cat allergen quantity tolerated in the baseline Controlled Cat Allergen Challenge as a covariate.

### Results

### Primary Endpoint

A single dose of REGN1908-1909 significantly increased the time to early asthmatic response (EAR) from 51 minutes (baseline) to >4 hours (maximum exposure) on days 8 (HR=0.36; P<0.0083), 29 (0.24; P<0.0001), 85 (0.27; P=0.0003) and to 232 mins on 57 (0.45; P=0.0222). A single dose of REGN1908-1909 also decreased the rate of EARs during cat allergen challenges within 8 days of treatment and for 3 months post dose. Thus, the primary endpoint was met. The median time to EAR could not be estimated in the REGN1908-1909 arm at most post-baseline visits, since a majority of patients were able to tolerate the full 4 hours of cat allergen exposure without experiencing an EAR (censored at 4 hours); therefore, the restricted mean survival times, which imputes censoring events as the maximum allowed time in EEU, are also provided as a post-hoc analysis, and are presented in the table as mean time in EEU in minutes.

TABLE 2
Time to Early Asthmatic Responds (EAR) During EEU Challenge

| | Median Time to EAR in Minutes | | Hazard Ratio | Mean Time in EEU | |
|---|---|---|---|---|---|
| | (95% CI) | | [p-value] | in Minutes | |
| | | REGN1908-1909 | REGN1908- | | REGN1908- |
| Study | Placebo | 600 mg | 1909 vs. | Placebo | 1909 600 mg |
| Day | (N = 27) | (N = 29) | placebo | (N = 27) | (N = 29) |
| Baseline | 51 (40.7, | 51 (31.3, 70.6) | | 56 | 52 |
| | 61.1) | | | | |
| Day 8 | 51 (33.9, | NR (130.9, NR) | 0.36 [0.0083] | 89 | 171 |
| (Primary) | 70.7) | | | | |
| Day 29 | 41 (31.4, | NR (131.2, NR) | 0.24 [<0.0001] | 71 | 176 |
| | 61.1) | | | | |
| Day 57 | 56 (40.5, | 232 (81.1, NR) | 0.45 [0.0222] | 92 | 171 |
| | 80.6) | | | | |
| Day 85 | 41 (31.0, | NR (100.0, NR) | 0.27 [0.0003] | 75 | 167 |
| | 60.9) | | | | |

NR = Not Reached (greater than 50% of patients had time to EAR censored at 4 hours)
CI = confidence interval

While all patients experienced an EAR within 2 hours during the baseline cat allergen challenge, the majority of patients receiving REGN1908-1909 did not experience an EAR within 4 hours during the cat allergen challenge 8 days after dosing. The instantaneous probability of experiencing an EAR during cat allergen exposure was reduced by 64% relative to placebo. A single dose of REGN1908-1909 significantly reduced the rate at which EARs occurred during cat allergen challenges 8, 29, 57, and 85 days after dosing relative to placebo.

Compared with placebo, REGN1908-1909 significantly increased the median time to EAR and decreased the rate of EAR within the 4 hours of the controlled cat allergen challenge, 7 days after treatment and for 3 months post-dose.

Furthermore, the single dose of REGN1908-1909 significantly reduced the incidence of EARs compared with placebo on days 8, 29, 57, and 85 (all P<0.05 except for day 57), as reflected in Table 3, below.

TABLE 3
Incidence of Early Asthmatic Responds (EAR) vs. Placebo

| | Patients experiencing an EAR | | |
|---|---|---|---|
| | | REGN1908-1909 | |
| Study | Placebo | 600 mg | P-value vs. |
| Day | (N = 27) | (N = 29) | placebo |
| Baseline | 27/27 (100%) | 29/29 (100%) | |
| Day 8 | 22/27 (81.5%) | 14/29 (48.3%) | 0.0128 |
| (Primary) | | | |
| Day 29 | 22/25 (88.0%) | 13/29 (44.8%) | 0.0014 |
| Day 57ª | 20/26 (76.9%) | 15/27 (55.6%) | 0.1480 |
| Day 85 | 21/24 (87.5%)) | 13/26 (50.0%) | 0.0063 |

ªEAR in REGN1908-1909 similar at all timepoints tested; EAR in placebo lower at day 57; EAR, early asthmatic response.

### Secondary Endpoints: Change in Patient-Reported Chest Symptoms

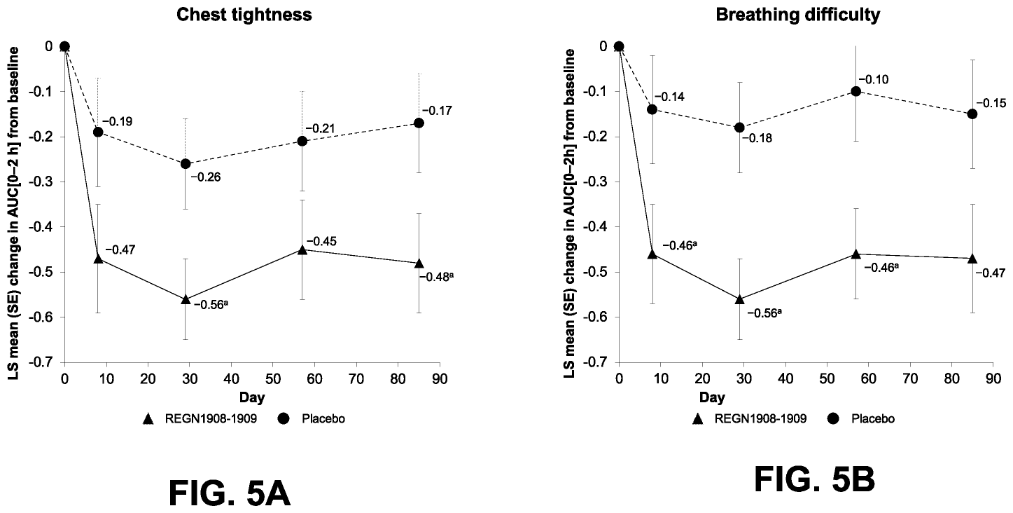

Patient-reported chest tightness was significantly reduced from baseline for subjects receiving single-dose REGN1908-1909 compared with placebo on Days 29 and 85 (FIG. 5A).

The change from baseline in patient-assessed breathing difficulty was significantly reduced in patients receiving REGN1908-1909 compared with placebo on Days 8, 29, and 57 (FIG. 5B).

### Secondary Endpoints: Change in FEV1 AUC (0-2 hr)

As compared with placebo, REGN1908-1909 significantly improved the AUC (0-2 hr) of acute FEV1 changes after cat allergen challenge in an EEU for 85 days after a single dose. In fact, as compared to baseline, REGN1908-1909 improved FEV1 AUC (0-2 hours) by day 8 (+15.2%) versus placebo (+1.6%, P<0.001). Thus, a secondary endpoint was met. The mean percent change from baseline in AUC (0-2 hr) of FEV1 is reflected in Table 4, below.

TABLE 4
Percent Change from Baseline in the AUC (0-2 hr)
of FEV1 with LOCF During EEU Challenges

| | | | Difference | p-value |
|---|---|---|---|---|
| | | REGN1908- | (Placebo vs. | (Placebo vs. |
| | | 1909 | REGN1908- | REGN1908- |
| Study Day | Placebo | 600 mg | 1909) | 1909) |
| Baseline | 2.65 | 2.71 | — | — |
| Mean AUC | | | | |
| of FEV1 | | | | |
| Day 8 | +1.6% | +15.2% | 13.6% | <0.001 |
| Day 29 | +0.5% | +16.7% | 16.2% | 0.002 |
| Day 57 | +1.8% | +14.1% | 12.3% | 0.016 |
| Day 85 | +0.2% | +12.7% | 12.5% | 0.008 |

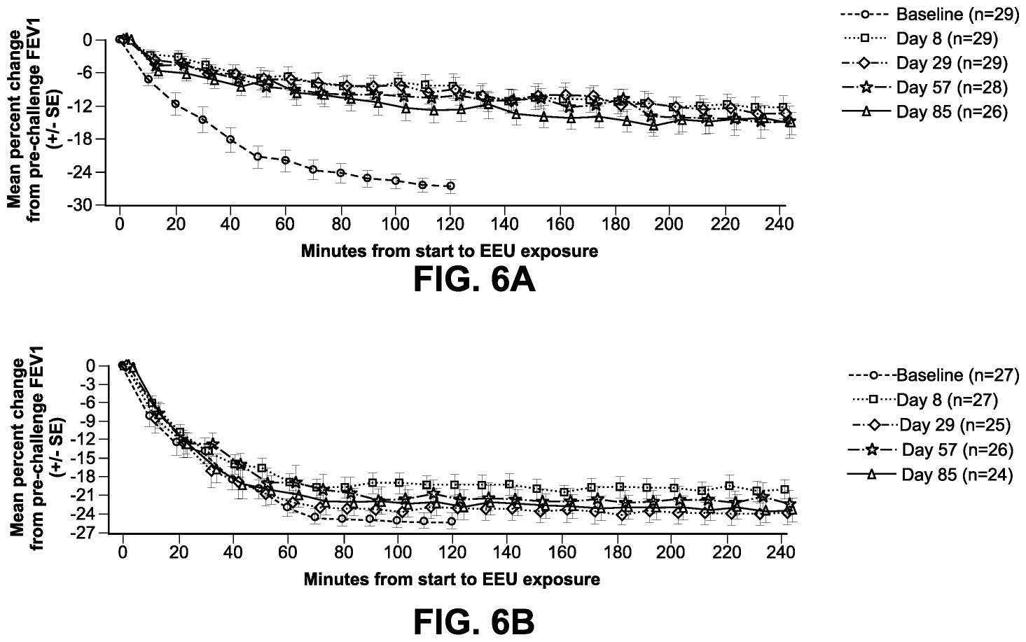

The AUC of FEV1 and percent change in FEV1 over the baseline cat allergen challenge were similar between placebo and REGN1908-1909 (FIGS. 6A and 6B). REGN 1908-1909 significantly prevented cat allergen-induced reductions in FEV1 over 0 to 2 hours during the controlled cat allergen challenges in an EEU, 7 days after treatment and for 3 months post dose. REGN1908-1909 significantly improved lung function during cat allergen exposure, as shown by an increase in the AUC of FEV1 during a cat allergen challenge as compared to placebo 8, 29, 57, and 85 days after a single dose. Indeed, REGN1908-1909 significantly prevented over about 70% of the lung function decline normally seen in such patients upon cat allergen exposure. Consistent results were seen whether looking at the percent change from baseline in the AUC of FEV1 or the change from baseline (data not shown).

### Secondary Endpoints: Change in Amount of Cat Allergen Tolerated

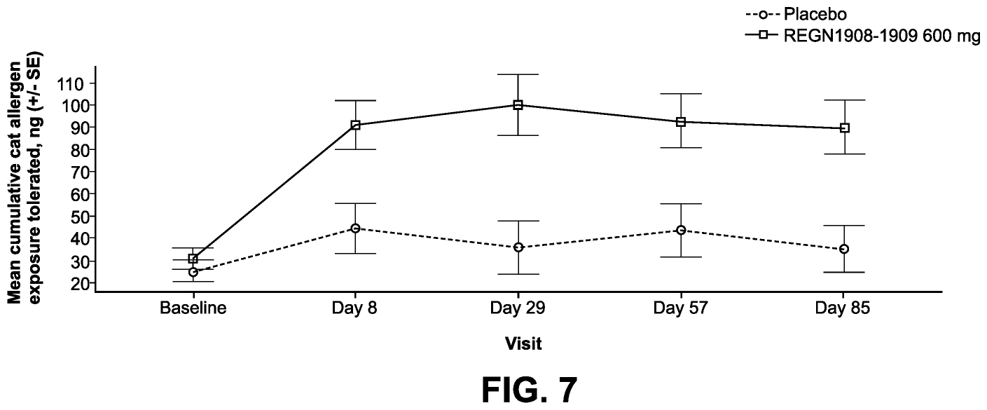

In addition to the time each patient tolerated in the EEU, when considering each individual patient's minute ventilation and the Fel d1 allergen concentration in the EEU that the patient was exposed to on a given day, there also was a significant increase from the baseline challenge in the cat allergen quantity tolerated by patients receiving REGN1908-1909 relative to placebo. Thus, another secondary endpoint was met. The mean change from baseline in cat allergen quantity tolerated by patients in nanograms (ng) (Fel d1 EEU allergen levels (ng/m³)×minute ventilation×time in EEU, where 1 L/min=0.001 m³/min) during cat allergen challenges is reflected in Table 5, below, and FIG. 7. When considering each individual patient's minute ventilation and the Fel d1 allergen concentration in the EEU, there was a significant increase from the baseline challenge in the cumulative cat allergen quantity tolerated by patients receiving REGN1908-1909 relative to placebo, 7 days after treatment and for 3 months post dose. By day 8, patients administered a single dose of REGN1908-1909 showed a 3-fold higher allergen quantity tolerated relative to baseline (60 nanograms (ng) REGN1908-1909 vs. 20 ng placebo, p=0.003).

TABLE 5

| | | | Difference | p-value |
|---|---|---|---|---|
| | | REGN1908- | (Placebo vs. | (Placebo vs. |
| | | 1909 | REGN1908- | REGN1908- |
| Study Day | Placebo | 600 mg | 1909) | 1909) |
| Baseline | 25.54 | 30.89 | — | — |
| Cumulative | | | | |
| Mean Allergen | | | | |
| Exposure | | | | |
| Tolerated (ng) | | | | |
| Day 8 | +19.55 | +59.55 | 39.50 | 0.003 |
| Day 29 | +14.14 | +68.21 | 54.7 | <0.001 |
| Day 57 | +21.94 | +55.38 | 33.44 | 0.023 |
| Day 85 | +12.93 | +54.03 | 41.10 | 0.003 |

The mean percent change from baseline in cat allergen quantity tolerated (Fel d1 EEU allergen levels×minute ventilation×time in EEU, where 1 L/min=0.001 m³/min) by patients during cat allergen challenges is reflected in Table 6, below.

TABLE 6

| | | | Difference | p-value |
|---|---|---|---|---|
| | | REGN1908- | Placebo vs. | (Placebo vs. |
| | | 1909 | REGN1908- | REGN1908- |
| Study Day | Placebo | 600 mg | 1909) | 1909) |
| Baseline Mean | 425.7 | 514.9 | — | — |
| Allergen | | | | |
| Quantity | | | | |
| Tolerated | | | | |
| Day 8 | +119.9% | +325.3% | 205.4% | 0.049 |
| Day 29 | +93.6% | +338.2% | 244.6% | 0.017 |
| Day 57 | +118.1% | +301.1% | 183.0% | 0.064 |
| Day 85 | +76.8% | +317.8% | 241.0% | 0.039 |

### Secondary Endpoints: Change in Skin Test Reactivity to Cat Allergen

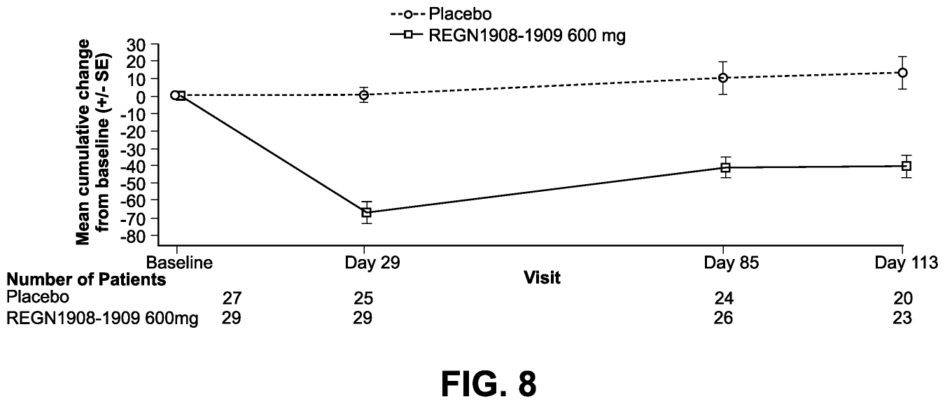

The effect of administration of a single dose of REGN-1908-1909 on skin reactivity to cat allergen was likewise evaluated. As shown in FIG. 8, a single dose of REGN1908-1909 reduced skin test reactivity to cat allergen at 1 week and for up to 4 months post dose, the last timepoint evaluated. Percent change from baseline AUC of skin prick with serial cat allergen were substantially and nominally significantly different between subjects randomized to REGN1908-1909 versus placebo. On day 29, LS mean (SE) percent change in AUC of SPT from baseline for subjects randomized to REGN1908-1909 was −66.70 (5.74)% versus 0.02 (6.19)% for subjects randomized to placebo (P<0.001).

### Secondary Endpoints: Change in Need for Rescue Medication

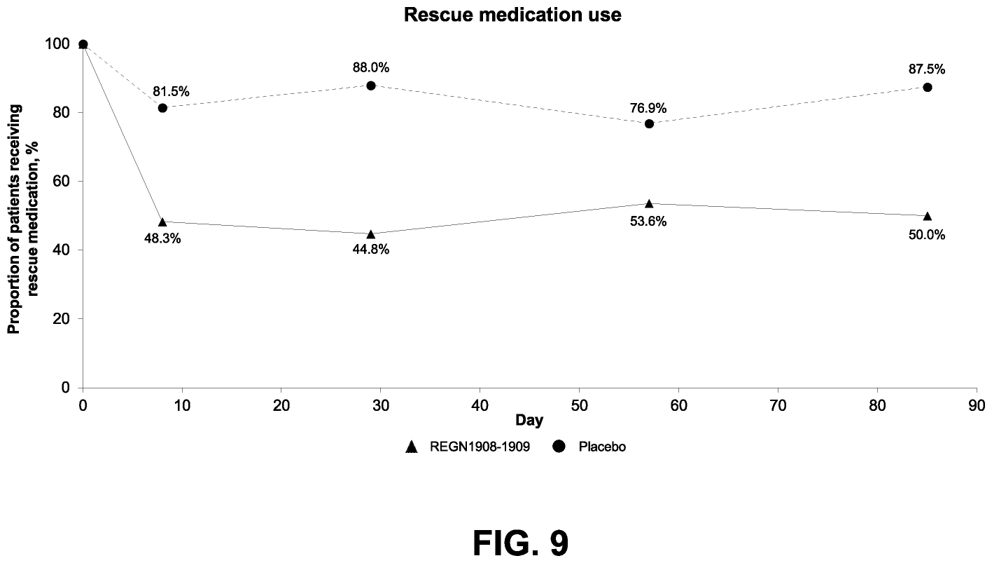

All 56 participants (100%) used a rescue medication at baseline at EEU exit (all participants had to have EAR during the 2-hour screening EEU; FIG. 9). During the observation period after baseline screening, 27.6% and 14.8% of REGN1908-1909- and placebo-treated subjects received rescue medications; during the at home monitoring period, 31% and 18.5% received rescue medications.

Post-treatment, more placebo-treated subjects received rescue medication upon EEU exit than REGN1908-1909 subjects, on Days 8, 29, 57, and 85, corresponding to a significant decrease in the incidence of EAR in the REGN1908-1909 subjects (FIG. 9). No difference in rescue medication use was observed between REGN1908-1909- and placebo-treated participants during the observation period, at home monitoring period, or at follow-up visits on Days 8, 29, 57, and 85.

### Secondary Endpoints: Change in TNSS and TOSS

Patient's nasal and ocular symptom scores elicited in the baseline Controlled Cat Allergen Challenge were not required to reach a certain threshold for inclusion in the study, as is typically required for EEU studies where an anti-allergic treatment is assessed to determine whether it reduces allergic rhinitis and conjunctivitis symptoms. During an EEU challenge, the drop in FEV1 often preceded the rise of nasal and ocular symptoms, such that subjects had to be removed from the EEU in response to their asthma exacerbation well before they exhibited robust allergic rhinitis and conjunctivitis symptoms.

No differences in the change in nasal and ocular symptoms from the baseline cat challenge, as measured by TNSS and TOSS, were observed in patients receiving REGN1908-1909 as compared to placebo. However, at the baseline cat allergen challenge, many patients experienced very mild or no measurable nasal or ocular symptoms. Typically, allergic rhinitis and conjunctivitis symptoms peak at 2-4 hours after allergen exposure in an EEU. Since patients experienced their asthma EAR at a median onset of <1 hour, they were removed from the chamber before the presentation of the peak nasal and ocular symptoms. The mean change from baseline in the AUC (0-2 hr) of TNSS during cat allergen challenges is reflected in Table 7, below.

TABLE 7

| | | | Difference | p-value |
|---|---|---|---|---|
| | | REGN1908- | (Placebo vs. | (Placebo vs. |
| | | 1909 | REGN1908- | REGN1908- |
| Study Day | Placebo | 600 mg | 1909) | 1909) |
| Mean TNSS | 3.40 | 2.84 | — | — allergic |
| AUC at | | | | rhinitis and |
| Baseline | | | | conjunctivitis |
| | | | | symptoms peak |
| | | | | at 2-4 hours after |
| | | | | allergen exposure |
| | | | | in an EEU |
| Day 8 | −0.71 | −0.49 | 0.22 | 0.675 |
| Day 29 | −0.70 | −1.39 | −0.68 | 0.182 |
| Day 57 | −0.93 | −0.83 | 0.10 | 0.866 |
| Day 85 | −0.49 | −1.37 | −0.88 | 0.146 |

The mean change from baseline in the AUC (0-2 hr) of TOSS during cat allergen challenges is reflected in Table 8, below.

TABLE 8

| | | | Difference | p-value |
|---|---|---|---|---|
| | | REGN1908- | (Placebo vs. | (Placebo vs. |
| | | 1909 | REGN1908- | REGN1908- |
| Study Day | Placebo | 600 mg | 1909) | 1909) |
| Mean TOSS | 0.74 | 0.56 | — | — |
| AUC at | | | | |
| Day 8 | −0.37 | −0.23 | 0.14 | 0.572 |
| Day 29 | −0.43 | −0.43 | 0.00 | 0.997 |
| Day 57 | −0.40 | −0.34 | 0.07 | 0.756 |
| Day 85 | −0.36 | −0.51 | -0.15 | 0.333 |

### Adverse Events

REGN1908-1909 was generally well tolerated. There were no serious adverse events (SAEs), no deaths, and no treatment emergent adverse events (TEAEs) leading to study discontinuation. The overall frequency of TEAEs and treatment-related TEAEs were comparable between REGN1908-1909 and placebo. All TEAEs were of mild or moderate severity, with no reports of deaths, SAEs, nor TEAEs leading to study discontinuation, and no significant injection site reactions (all mild in severity). Two subjects (6.9%) receiving REGN1908-1909 experienced injection site reactions compared to one subject (3.7%) receiving placebo; all were mild in severity. One female subject became pregnant during the study as she was using abstinence as contraception. She received REGN1908-1909 and is in her third trimester at the time of writing the KRM.

### Pharmacokinetic Results

REGN1908-1909 exhibited concentration time profiles consistent with expected profiles from previous studies. Furthermore, the concentration time profile is characterized by an initial absorption phase and mono-exponential elimination. Based on these similarities, it can be implied that the kinetics of REGN1908-1909 in cat allergic patients is not dose dependent and exhibits linearity. The half-life of REGN1908 and REGN1909 was 28.1±4.3 days and 21.3±3.6 days, respectively, comparable to half-life measured in earlier studies. Concentration time profiles in serum of REGN1908-1909 were comparable to previous studies in healthy subjects and cat allergic subjects

### Summary of Results

Thus, a single dose of REGN1908-1909 rapidly and durably reduced acute bronchoconstriction and increased the time to early asthmatic response (EAR) in cat-allergic patients with mild asthma upon exposure to cat allergen in an EEU within 8 days post dose and consistent through 85 days post dose. Acute bronchoconstriction upon exposure to allergen in the susceptible asthmatic airway results from acute mast cell degranulation and release of preformed mediators.

This early phase response may then be followed hours later by a late phase reduction in FEV1, thought to occur due to an influx of multiple cells and inflammatory cytokines, most representative of the chronic asthmatic airway. Indeed, consistent results were seen in an improvement in lung function as measured by FEV1 and an increase in the cat allergen quantity tolerated by patients upon cat allergen exposure after a single dose of REGN1908-1909 relative to placebo out to 3 months. In the cat allergen challenges, asthma responses precluded nasal and ocular symptoms; therefore, an effect of REGN1908-1909 on TNSS and TOSS could not be detected in the context of this study design.

Though not a primary or secondary endpoint in this study, REGN1908-1909 did not have a clear effect on the improvement in the late phase reduction in FEV1, which was experienced by approximately 30% of subjects in this study (data not shown). However, a single dose of REGN1908-1909 potently and durably inhibited early asthmatic responses to cat allergen. REGN1908-1909 was generally well tolerated. There were no deaths or TEAEs leading to study discontinuation. The concentration time profiles of REGN1908-1909 exhibited linear PK, with half-lives of each antibody described above.

### CONCLUSION

Single-dose REGN1908-1909 significantly reduced acute bronchoconstriction and increased the time to EAR in cat-allergic mild asthmatic subjects upon EEU exposure to cat allergen at 8 days and up to 3 months post-dose. A single administration of REGN1908-1909 controlled patients' allergic response to cat allergen, preventing early asthma reactions for the duration of the 3-month trial. Patients experienced significant improvements in lung function and cat allergen tolerance from the first assessment at week 1.

In cat-allergic mild asthmatic subjects exposed to aerosolized cat allergen in an EEU, a single dose of subcutaneous REGN1908-1909 600 mg was shown to result in a durable effect versus placebo, significantly reducing the incidence of early asthmatic responses, reducing chest symptoms, and reducing the use of rescue medication up to 3 months post dose. In addition, REGN1908-1909 was generally well tolerated. REGN1908-1909 is being evaluated as an alternative to subcutaneous allergy immunotherapy for allergic rhinitis and allergic asthma related to cat allergy. Benefits of REGN1908-1909 include its fast onset of action, its durability, and its favorable safety profile.

What is claimed is:

1. A method for increasing the time to early asthmatic response (EAR) upon exposure to a Fel d1 allergen in a patient having an allergy to the Fel d1 allergen, the method comprising administering to the patient one or more doses of each of a first Fel d1-specific monoclonal antibody or antigen-binding fragment thereof and a second Fel d1-specific monoclonal antibody or antigen-binding fragment thereof, wherein the first Fel d1-specific monoclonal antibody or antigen-binding fragment thereof comprises a heavy chain complementarity determining region 1 (HCDR1) having the amino acid sequence of SEQ ID NO:20, an HCDR2 having the amino acid sequence of SEQ ID NO:22, an HCDR3 having the amino acid sequence of SEQ ID N0:24, a light chain complementarity determining region 1 (LCDR1) having the amino acid sequence of SEO ID NO:28, an LCDR2 having the amino acid sequence of SEQ ID NO:30, and an LCDR3 having the amino acid sequence of SEQ II) NO:32, and wherein the second Fel d1-specific monoclonal antibody or antigen-binding fragment thereof comprises an HCDR1 having the amino acid sequence of SEQ ID NO:308, an HCDR2 having the amino acid sequence of SEQ ID NO:310, an HCDR3 having the amino acid sequence of SEQ ID NO:312, an LCDR1 having the amino acid sequence of SEQ ID NO:316, an LCDR2 having the amino acid sequence of SEQ ID NO:318, and an LCDR having the amino acid sequence of SEQ ID NO:320; wherein the subject has cat allergen-triggered asthma.

2. The method of claim 1, wherein the patient is a cat-allergic patient not living with a cat.

3. The method of claim 1, wherein the incidence of EAR is reduced by about 20% to about 50% after administration of the first Fel d1-specific monoclonal antibody or antigen-binding fragment thereof and the second Fel d1-specific monoclonal antibody or antigen-binding fragment thereof; and/or the incidence of EAR is reduced for at least 56 days after administration of the first Fel d1-specific monoclonal antibody or antigen-binding fragment thereof and the second Fel d1-specific monoclonal antibody or antigen-binding fragment thereof.

4. (canceled)

5. The method of claim 1, wherein patient-reported chest tightness and/or patient-reported breathing difficulty is reduced.

6. (canceled)

7. The method of claim 1, wherein the patient has a reduced likelihood of needing rescue medication upon exposure to the Fel d1 allergen for up to at least about three months after administration of the first Fel d1-specific monoclonal antibody or antigen-binding fragment thereof and the second Fel d1-specific monoclonal antibody or antigen-binding fragment thereof.

8. The method of claim 1, wherein the time to EAR is increased to greater than 4 hours.

9. The method of claim 1, wherein the patient has mild asthma with rhinitis.

10-13. (canceled)

14. A method for improving lung function and/or for reducing acute bronchoconstriction upon exposure to a Fel d1 allergen in a patient having mild asthma and an allergy to the Fel d1 allergen, the method comprising administering to the patient one or more doses of each of a first Fel d1-specific monoclonal antibody or antigen-binding fragment thereof and a second Fel d1-specific monoclonal antibody or antigen-binding fragment thereof, wherein the first Fel d1-specific monoclonal antibody or antigen-binding fragment thereof comprises a heavy chain complementarity determining region 1 (HCDR1) having the amino acid sequence of SEQ ID NO:20, an HCDR2 having the amino acid sequence of SEQ ID NO:22, an HCDR3 having the amino acid sequence of SEQ ID NO:24, a light chain complementarity determining region 1 (LCDR1) having the amino acid sequence of SEQ II) NO:28, an LCDR2 having the amino acid sequence of SEQ ID NO:30, and an LCDR3 having the amino acid sequence of SEQ ID NO:32, and wherein the second Fel d1-specific monoclonal antibody or antigen-binding fragment thereof comprises an HCDR1 having the amino acid sequence of SEQ ID NO:308, an HCDR2 having the amino acid sequence of SEQ ID NO:310, an HCDR3 having die amino acid sequence of SEQ ID NO:312, an LCDR1 having the amino acid sequence of SEQ ID NO:316, an LCDR2 having the amino acid sequence of SEQ ID NO:318, and an LCDR having the amino acid sequence of SEQ ID NO:320.

15. The method of claim 14, wherein the patient has mild asthma with rhinitis.

16. The method of claim 14, wherein the improvement in lung function comprises an increase in the forced expiratory volume (FEV1) area under curve (AUC) of the patient.

17. The method of claim 16, wherein the increase in FEV1 AUC is at least about 12%.

18-20. (canceled)

21. The method of claim 1, wherein the first Fel d1-specific monoclonal antibody or antigen-binding fragment thereof comprises a heavy chain variable region (HCVR) having the amino acid sequence of SEQ ID NO: 18 and a light chain variable region (LCVR) having the amino acid sequence of SEO II) NO:26.

22. The method of claim 1, wherein the second Fel d1-specific monoclonal antibody or antigen-binding fragment thereof comprises a heavy chain variable region (HCVR) having the amino acid sequence of SEQ ID NO:306 and a light chain variable region (LCVR) having the amino acid sequence of SEQ ID NO. 314.

23. (canceled)

24. (canceled)

25. The method of claim 1, wherein the first Fel d1-specific monoclonal antibody or antigen-binding fragment thereof and the second Fel d1-specific monoclonal antibody or antigen-binding fragment thereof are administered subcutaneously, intravenously, or intranasally to the patient.

26. The method of claim 1, wherein the first Fel d1-specific monoclonal antibody or antigen-binding fragment thereof and the second Fel d1-specific monoclonal antibody or antigen-binding fragment thereof are administered sequentially or concurrently to the subject.

27. The method of claim 26, wherein the first Fel d1-specific monoclonal antibody or antigen-binding fragment thereof and the second Fel d1-specific monoclonal antibody or antigen-binding fragment thereof are administered concurrently to the subject.

28. (canceled)

29. The method of claim 1, wherein each of the first Fel d1-specific monoclonal antibody or antigen-binding fragment thereof and the second Fel d1-specific monoclonal antibody or antigen-binding fragment thereof are administered subcutaneously, each at a dose of about 300 mg.

30. The method of claim 1, wherein a single dose of each of the first Fel d1-specific monoclonal antibody or antigen-binding fragment thereof and the second Fel d1-specific monoclonal antibody or antigen-binding fragment thereof is administered to the patient.

31. The method of claim 1, wherein the first Fel d1-specific monoclonal antibody or antigen-binding fragment thereof and the second Fel d1-specific monoclonal antibody or antigen-binding fragment thereof are administered to the patient every 12 weeks.

32. The method of claim 1, wherein the first Fel d1-specific monoclonal antibody or antigen-binding fragment thereof and the second Fel d1-specific monoclonal antibody or antigen-binding fragment thereof are co-formulated.

33. The method of claim 1, wherein the first Fel d1-specific monoclonal antibody or antigen-binding fragment thereof and the second Fel d1-specific monoclonal antibody or antigen-binding fragment thereof are formulated separately.


SEQUENCE LISTING (491 sequences)

SEQ ID NO: 1 - DNA, 363 bp, synthetic construct
  source 1..363: mol_type = other DNA
gaggtgcagctggtggagtctgggggaggcttggtccagcctggggggtccctgagactctcctgtacagcctctggattcatttttagtaacttttggatgacttgggtccgccaggctccagggaaggggctggagtgggtggccaacataaagcaagatggaagtgagacatactgtgtggactctgtgaagggccgattcaccatctccagagaccacgccaagaactcactgtttctgcaaatgaacagcctgagagccgaggacacggctgtatattactgtgcaagaggagctggaacaatctactactacggtatggacgtctggggccaagggaccacggtcattgtctcctca

SEQ ID NO: 2 - protein, 121 aa, synthetic construct
EVQLVESGGGLVQPGGSLRLSCTASGFIFSNFWMTWVRQAPGKGLEWVANIKQDGSETYCVDSVKGRFTISRDHAKNSLFLQMNSLRAEDTAVYYCARGAGTIYYYGMDVWGQGTTVIVSS

SEQ ID NO: 3 - DNA, 24 bp, synthetic construct
  source 1..24: mol_type = other DNA
ggattcatttttagtaacttttgg

SEQ ID NO: 4 - protein, 8 aa, synthetic construct
GFIFSNFW

SEQ ID NO: 5 - DNA, 24 bp, synthetic construct
  source 1..24: mol_type = other DNA
ataaagcaagatggaagtgagaca

SEQ ID NO: 6 - protein, 8 aa, synthetic construct
IKQDGSET

SEQ ID NO: 7 - DNA, 42 bp, synthetic construct
  source 1..42: mol_type = other DNA
gcaagaggagctggaacaatctactactacggtatggacgtc

SEQ ID NO: 8 - protein, 14 aa, synthetic construct
ARGAGTIYYYGMDV

SEQ ID NO: 9 - DNA, 321 bp, synthetic construct
  source 1..321: mol_type = other DNA
gacatccagatgacccagtctccatcttccgtgtctgcatctgtaggagacagactcaccatcacttgtcgggcgagtcaggatattagcagctggttaacctggtatcagcagaaaccagggaaagcccctaacctcctgatctatgctgcatccggtttgcaaagtggagtcccatcaaggttcagcggcagtggatctgggacagatttcactctcaccatcagcaacctgcagcctgaagattttgcaacttactattgtcaacaggctaacagtttcccgctcaccttcggccaagggacacgactggagattaaa

SEQ ID NO: 10 - protein, 107 aa, synthetic construct
DIQMTQSPSSVSASVGDRLTITCRASQDISSWLTWYQQKPGKAPNLLIYAASGLQSGVPSRFSGSGSGTDFTLTISNLQPEDFATYYCQQANSFPLTFGQGTRLEIK

SEQ ID NO: 11 - DNA, 18 bp, synthetic construct
  source 1..18: mol_type = other DNA
caggatattagcagctgg

SEQ ID NO: 12 - protein, 6 aa, synthetic construct
QDISSW

SEQ ID NO: 13-14 - skipped (2 entries)

SEQ ID NO: 15 - DNA, 27 bp, synthetic construct
  source 1..27: mol_type = other DNA
caacaggctaacagtttcccgctcacc

SEQ ID NO: 16 - protein, 9 aa, synthetic construct
QQANSFPLT

SEQ ID NO: 17 - DNA, 357 bp, synthetic construct
  source 1..357: mol_type = other DNA
gaggtgcagctggtggagtctgggggaggcctggtcaagcctggggggtccctgagactctcctgtgcagcctctggattcaccttcagaaactataacataaactgggtccgccaggctccagggaaggggctggagtgggtctcactcatcagtggtagtagtagttacatatattacgcagactcagtgaagggccgattcaccgtctccagagacaacgccaagaactcactgtatctgcaaatgaacagcctgagagccgaggacacggctgtgtattactgtgcgaggcggacattaagctactacgttatggacgtctggggccaagggaccacggtcaccgtctcctca

SEQ ID NO: 18 - protein, 119 aa, synthetic construct
EVQLVESGGGLVKPGGSLRLSCAASGFTFRNYNINWVRQAPGKGLEWVSLISGSSSYIYYADSVKGRFTVSRDNAKNSLYLQMNSLRAEDTAVYYCARRTLSYYVMDVWGQGTTVTVSS

SEQ ID NO: 19 - DNA, 24 bp, synthetic construct
  source 1..24: mol_type = other DNA
ggattcaccttcagaaactataac

SEQ ID NO: 20 - protein, 8 aa, synthetic construct
GFTFRNYN

SEQ ID NO: 21 - DNA, 24 bp, synthetic construct
  source 1..24: mol_type = other DNA
atcagtggtagtagtagttacata

SEQ ID NO: 22 - protein, 8 aa, synthetic construct
ISGSSSYI

SEQ ID NO: 23 - DNA, 36 bp, synthetic construct
  source 1..36: mol_type = other DNA
gcgaggcggacattaagctactacgttatggacgtc

SEQ ID NO: 24 - protein, 12 aa, synthetic construct
ARRTLSYYVMDV

SEQ ID NO: 25 - DNA, 321 bp, synthetic construct
  source 1..321: mol_type = other DNA
gacatccaggtgacccagtctccatcccccctgtctgcatctgtaggagacagagtcaccatcacttgccgggcgagtcagggcattagcaattatttagcctggtatcagcagaaaccagggagagttcctcagctcctgatctatgctgcatccactttgcaatcaggggtcccatctcggttcagtggcagtggatctgggacagatttcactctcaccatcagcagcctgcagcctgaagatgttgcaacttattactgtcaaaagtataacagtgccccgtacacttttggccaggggaccaagctggagatcaaa

SEQ ID NO: 26 - protein, 107 aa, synthetic construct
DIQVTQSPSPLSASVGDRVTITCRASQGISNYLAWYQQKPGRVPQLLIYAASTLQSGVPSRFSGSGSGTDFTLTISSLQPEDVATYYCQKYNSAPYTFGQGTKLEIK

SEQ ID NO: 27 - DNA, 18 bp, synthetic construct
  source 1..18: mol_type = other DNA
cagggcattagcaattat

SEQ ID NO: 28 - protein, 6 aa, synthetic construct
QGISNY

SEQ ID NO: 29-30 - skipped (2 entries)

SEQ ID NO: 31 - DNA, 27 bp, synthetic construct
  source 1..27: mol_type = other DNA
caaaagtataacagtgccccgtacact

SEQ ID NO: 32 - protein, 9 aa, synthetic construct
QKYNSAPYT

SEQ ID NO: 33 - DNA, 363 bp, synthetic construct
  source 1..363: mol_type = other DNA
caggtacagctgcagcagtcaggtccaggactggtgaagtcctcgcagaccctctcactcacctgtgccatctccggggacagtgtctctagcaacagtgttgcttggaattggatcaggcagtccccatcgagaggccttgagtggctggggaggacatactacaggtccaaatggtataatgattatgcagtatctgtgaaaagtcgaataaacatcaacccagacacatccaagaaccacttctccctgcagttgaattctgtgactcccgaggacacggctgtttatttctgtgcaagagcctggaactggtactaccttgactactggggccagggcaccctggtcaccgtctcgtca

SEQ ID NO: 34 - protein, 121 aa, synthetic construct
QVQLQQSGPGLVKSSQTLSLTCAISGDSVSSNSVAWNWIRQSPSRGLEWLGRTYYRSKWYNDYAVSVKSRININPDTSKNHFSLQLNSVTPEDTAVYFCARAWNWYYLDYWGQGTLVTVSS

SEQ ID NO: 35 - DNA, 30 bp, synthetic construct
  source 1..30: mol_type = other DNA
ggggacagtgtctctagcaacagtgttgct

SEQ ID NO: 36 - protein, 10 aa, synthetic construct
GDSVSSNSVA

SEQ ID NO: 37 - DNA, 27 bp, synthetic construct
  source 1..27: mol_type = other DNA
acatactacaggtccaaatggtataat

SEQ ID NO: 38 - protein, 9 aa, synthetic construct
TYYRSKWYN

SEQ ID NO: 39 - DNA, 33 bp, synthetic construct
  source 1..33: mol_type = other DNA
gcaagagcctggaactggtactaccttgactac

SEQ ID NO: 40 - protein, 11 aa, synthetic construct
ARAWNWYYLDY

SEQ ID NO: 41 - DNA, 339 bp, synthetic construct
  source 1..339: mol_type = other DNA
gatattgtgatgacccagtctccagactccctggctatgtctctaggcgagagggccaccatcaactgcaagtccagccagagtgttttatacagctccaacaataagaattacttaggttggtaccagcagaaaccaggacagcctcctaaactgctcatttactgggcatctacccgggaatccggtgtccctgaccgaatcagtggcagcgggtctgggacagatttcactctcaccatcagcagcctgcaggctgaagatgtggcagtttattactgtcagcaatatcttagaaatacgctcactttcggcggagggaccaaggtggagatcaaa

SEQ ID NO: 42 - protein, 113 aa, synthetic construct
DIVMTQSPDSLAMSLGERATINCKSSQSVLYSSNNKNYLGWYQQKPGQPPKLLIYWASTRESGVPDRISGSGSGTDFTLTISSLQAEDVAVYYCQQYLRNTLTFGGGTKVEIK

SEQ ID NO: 43 - DNA, 36 bp, synthetic construct
  source 1..36: mol_type = other DNA
cagagtgttttatacagctccaacaataagaattac

SEQ ID NO: 44 - protein, 12 aa, synthetic construct
QSVLYSSNNKNY

SEQ ID NO: 45-46 - skipped (2 entries)

SEQ ID NO: 47 - DNA, 27 bp, synthetic construct
  source 1..27: mol_type = other DNA
cagcaatatcttagaaatacgctcact

SEQ ID NO: 48 - protein, 9 aa, synthetic construct
QQYLRNTLT

SEQ ID NO: 49 - DNA, 381 bp, synthetic construct
  source 1..381: mol_type = other DNA
gaggtgcagctggtggagtctgggggaggcctggtcaagcctggggggtccctgagactctcctgtgcagcctctggattcaccttcagtagctatagcatgaactgggtccgccaggctccagggaaggggctggagtgggtctcatccattagtagtagaagtagttacatatactacgcagactcagtgaagggccgattcaccatctcaagagacaacgccaagaactcactgtatctgcaaatgaacagcctgagagccgaggacacggctgtgtattattgtgcgagagatcatattgtagtagtaccaggtgcctcctactactactacggtatggacgtctggggccaagggaccacggtcaccgtctcctca

SEQ ID NO: 50 - protein, 127 aa, synthetic construct
EVQLVESGGGLVKPGGSLRLSCAASGFTFSSYSMNWVRQAPGKGLEWVSSISSRSSYIYYADSVKGRFTISRDNAKNSLYLQMNSLRAEDTAVYYCARDHIVVVPGASYYYYGMDVWGQGTTVTVSS

SEQ ID NO: 51 - DNA, 24 bp, synthetic construct
  source 1..24: mol_type = other DNA
ggattcaccttcagtagctatagc

SEQ ID NO: 52 - protein, 8 aa, synthetic construct
GFTFSSYS

SEQ ID NO: 53 - DNA, 24 bp, synthetic construct
  source 1..24: mol_type = other DNA
attagtagtagaagtagttacata

SEQ ID NO: 54 - protein, 8 aa, synthetic construct
ISSRSSYI

SEQ ID NO: 55 - DNA, 60 bp, synthetic construct
  source 1..60: mol_type = other DNA
gcgagagatcatattgtagtagtaccaggtgcctcctactactactacggtatggacgtc

SEQ ID NO: 56 - protein, 20 aa, synthetic construct
ARDHIVVVPGASYYYYGMDV

SEQ ID NO: 57 - DNA, 321 bp, synthetic construct
  source 1..321: mol_type = other DNA
gacatccagatgacccagtctccatcctccctgtctgcatctgtaggagacagagtcaccatcacttgccgggcaagtcagggcattagaaatgatttaggctggtatcagcagaaaccagggaaagcccctaagcgcctgatctctgctgcatccagtttgcaaagtggggtcccatcaaggttcagcggcagtggatctgggacagaattcactctcacaatcagcagcctgcagcctgaagattttgcaacttattactgtctacagcataatagttacccgctcactttcggcggagggaccaaggtggagatcaaa

SEQ ID NO: 58 - protein, 107 aa, synthetic construct
DIQMTQSPSSLSASVGDRVTITCRASQGIRNDLGWYQQKPGKAPKRLISAASSLQSGVPSRFSGSGSGTEFTLTISSLQPEDFATYYCLQHNSYPLTFGGGTKVEIK

SEQ ID NO: 59 - DNA, 18 bp, synthetic construct
  source 1..18: mol_type = other DNA
cagggcattagaaatgat

SEQ ID NO: 60 - protein, 6 aa, synthetic construct
QGIRND

SEQ ID NO: 61-62 - skipped (2 entries)

SEQ ID NO: 63 - DNA, 27 bp, synthetic construct
  source 1..27: mol_type = other DNA
ctacagcataatagttacccgctcact

SEQ ID NO: 64 - protein, 9 aa, synthetic construct
LQHNSYPLT

SEQ ID NO: 65 - DNA, 369 bp, synthetic construct
  source 1..369: mol_type = other DNA
caggtgcagctggtgcagtctggggctgaggtgaagaagcctggggcctcagtggaggtctcctgcaaggcttctggatacaccttcaccgactactatatacactggatacgacaggcccctggacaagggcttgagtggatgggatggatcaaccctgacagtggtcgcacaaactatgcacagaagtttcaggtcagggtcaccatgaccagggacacgtccatcaccacagcctacatggaactgaacagactgaaatctgacgacacggccgtgtattactgtgcgagaggacccctacgtggatatagcggctacgatttttttgactactggggccagggaaccctggtcaccgtctcctca

SEQ ID NO: 66 - protein, 123 aa, synthetic construct
QVQLVQSGAEVKKPGASVEVSCKASGYTFTDYYIHWIRQAPGQGLEWMGWINPDSGRTNYAQKFQVRVTMTRDTSITTAYMELNRLKSDDTAVYYCARGPLRGYSGYDFFDYWGQGTLVTVSS

SEQ ID NO: 67 - DNA, 24 bp, synthetic construct
  source 1..24: mol_type = other DNA
ggatacaccttcaccgactactat

SEQ ID NO: 68 - protein, 8 aa, synthetic construct
GYTFTDYY

SEQ ID NO: 69 - DNA, 24 bp, synthetic construct
  source 1..24: mol_type = other DNA
atcaaccctgacagtggtcgcaca

SEQ ID NO: 70 - protein, 8 aa, synthetic construct
INPDSGRT

SEQ ID NO: 71 - DNA, 48 bp, synthetic construct
  source 1..48: mol_type = other DNA
gcgagaggacccctacgtggatatagcggctacgatttttttgactac

SEQ ID NO: 72 - protein, 16 aa, synthetic construct
ARGPLRGYSGYDFFDY

SEQ ID NO: 73 - DNA, 339 bp, synthetic construct
  source 1..339: mol_type = other DNA
gacatcgtgatgacccagtctccagactccctggctatatctctgggcgagagggccaccatcaactgcaagtccagccagagtgttttatacagctccaacaataagcagtacttagcttggtacaagcagagaccaggacagcctcctaagctgctcatttcctggacatctacccgggaatccggggtccctgaccgattcagtggcagcgggtctgggacagatttcactctcaccatcagcagcctgcaggctgaagatgtggcagtttatttctgtcaacaatattatagtactccgtacacttttggccaggggaccaagctggagatcaga

SEQ ID NO: 74 - protein, 113 aa, synthetic construct
DIVMTQSPDSLAISLGERATINCKSSQSVLYSSNNKQYLAWYKQRPGQPPKLLISWTSTRESGVPDRFSGSGSGTDFTLTISSLQAEDVAVYFCQQYYSTPYTFGQGTKLEIR

SEQ ID NO: 75 - DNA, 36 bp, synthetic construct
  source 1..36: mol_type = other DNA
cagagtgttttatacagctccaacaataagcagtac

SEQ ID NO: 76 - protein, 12 aa, synthetic construct
QSVLYSSNNKQY

SEQ ID NO: 77-78 - skipped (2 entries)

SEQ ID NO: 79 - DNA, 27 bp, synthetic construct
  source 1..27: mol_type = other DNA
caacaatattatagtactccgtacact

SEQ ID NO: 80 - protein, 9 aa, synthetic construct
QQYYSTPYT

SEQ ID NO: 81 - DNA, 369 bp, synthetic construct
  source 1..369: mol_type = other DNA
caggtggtactggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatggtatgatggaagtaataaatactatgtagactccgtgaagggccgattcaccatctccagagacaattccaataacacaatctatctgcaaatgaacagcctgagagccgaggacacggctgtatattactgtgcgagatcccttataccagtggctggtacggaccccatttttggatactggggccagggaaccctggtcaccgtctcctca

SEQ ID NO: 82 - protein, 123 aa, synthetic construct
QVVLVESGGGVVQPGRSLRLSCAASGFTFSSYGMHWVRQAPGKGLEWVAVIWYDGSNKYYVDSVKGRFTISRDNSNNTIYLQMNSLRAEDTAVYYCARSLIPVAGTDPIFGYWGQGTLVTVSS

SEQ ID NO: 83 - DNA, 24 bp, synthetic construct
  source 1..24: mol_type = other DNA
ggattcaccttcagtagctatggc

SEQ ID NO: 84 - protein, 8 aa, synthetic construct
GFTFSSYG

SEQ ID NO: 85 - DNA, 24 bp, synthetic construct
  source 1..24: mol_type = other DNA
atatggtatgatggaagtaataaa

SEQ ID NO: 86 - protein, 8 aa, synthetic construct
IWYDGSNK

SEQ ID NO: 87 - DNA, 48 bp, synthetic construct
  source 1..48: mol_type = other DNA
gcgagatcccttataccagtggctggtacggaccccatttttggatac

SEQ ID NO: 88 - protein, 16 aa, synthetic construct
ARSLIPVAGTDPIFGY

SEQ ID NO: 89 - DNA, 321 bp, synthetic construct
  source 1..321: mol_type = other DNA
gacatccagatgacccagtctccttccaccctgtctgcatctgtaggagacagagtcaccatcacttgccgggccagtcagagtgttagtagctggttggcctggtatcagcagaaaccagggaaagcccctaaactcctgatctttaaggcgtctggtttagaaagtggggtcccatttaggttcagcggcagtggatctgggacagaattcactctcaccatcagcagcctgcagcctgatgattttgcaacttattactgccaacagtataatacttattctccgacgttcggccaagggaccaaggtggagatcaaa

SEQ ID NO: 90 - protein, 107 aa, synthetic construct
DIQMTQSPSTLSASVGDRVTITCRASQSVSSWLAWYQQKPGKAPKLLIFKASGLESGVPFRFSGSGSGTEFTLTISSLQPDDFATYYCQQYNTYSPTFGQGTKVEIK

SEQ ID NO: 91 - DNA, 18 bp, synthetic construct
  source 1..18: mol_type = other DNA
cagagtgttagtagctgg

SEQ ID NO: 92 - protein, 6 aa, synthetic construct
QSVSSW

SEQ ID NO: 93-94 - skipped (2 entries)

SEQ ID NO: 95 - DNA, 27 bp, synthetic construct
  source 1..27: mol_type = other DNA
caacagtataatacttattctccgacg

SEQ ID NO: 96 - protein, 9 aa, synthetic construct
QQYNTYSPT

SEQ ID NO: 97 - DNA, 360 bp, synthetic construct
  source 1..360: mol_type = other DNA
caggtgcagctgcaggagtcgggcccaggactggtgaagccttcggagaccctgtccctcacctgcactgtctctggtggctccatcagtaattactactggagctggatccggcagcccccagggaagggactggagtggattggatatatctattatagtgggagaaccaactacaacccctccctcaagagtcgagtcaccatatcagtggacacgtccaagaaccagttctccctgaagctgaggtctgtgaccgccgcagacacggccgtgtattactgtgcgagacatcgtataactagaactgcggactcctttgactactggggccagggaaccctggtcaccgtctcctca

SEQ ID NO: 98 - protein, 120 aa, synthetic construct
QVQLQESGPGLVKPSETLSLTCTVSGGSISNYYWSWIRQPPGKGLEWIGYIYYSGRTNYNPSLKSRVTISVDTSKNQFSLKLRSVTAADTAVYYCARHRITRTADSFDYWGQGTLVTVSS

SEQ ID NO: 99 - DNA, 24 bp, synthetic construct
  source 1..24: mol_type = other DNA
ggtggctccatcagtaattactac

SEQ ID NO: 100 - protein, 8 aa, synthetic construct
GGSISNYY

SEQ ID NO: 101 - DNA, 21 bp, synthetic construct
  source 1..21: mol_type = other DNA
atctattatagtgggagaacc

SEQ ID NO: 102 - protein, 7 aa, synthetic construct
IYYSGRT

SEQ ID NO: 103 - DNA, 42 bp, synthetic construct
  source 1..42: mol_type = other DNA
gcgagacatcgtataactagaactgcggactcctttgactac

SEQ ID NO: 104 - protein, 14 aa, synthetic construct
ARHRITRTADSFDY

SEQ ID NO: 105 - DNA, 321 bp, synthetic construct
  source 1..321: mol_type = other DNA
gacatccagatgacccagtctccatcctccctgtctgcatctgtaggagacagagtcaccatcacttgccaggcgagtcaggacattaccaactatttaaattggtatcagcagaaaccagggaaagcccctaagctcctgatctacgatgcatccaatttggaaacaggggtcccatcaaggttcagtggaagtggatctgggacagattttactttcaccatcagcagcctgcagcctgaagatgttgcaacattttactgtcaccagtatggtgatctcccgtacacttttggccaggggaccaagctggagatcaaa

SEQ ID NO: 106 - protein, 107 aa, synthetic construct
DIQMTQSPSSLSASVGDRVTITCQASQDITNYLNWYQQKPGKAPKLLIYDASNLETGVPSRFSGSGSGTDFTFTISSLQPEDVATFYCHQYGDLPYTFGQGTKLEIK

SEQ ID NO: 107 - DNA, 18 bp, synthetic construct
  source 1..18: mol_type = other DNA
caggacattaccaactat

SEQ ID NO: 108 - protein, 6 aa, synthetic construct
QDITNY

SEQ ID NO: 109-110 - skipped (2 entries)

SEQ ID NO: 111 - DNA, 27 bp, synthetic construct
  source 1..27: mol_type = other DNA
caccagtatggtgatctcccgtacact

SEQ ID NO: 112 - protein, 9 aa, synthetic construct
HQYGDLPYT

SEQ ID NO: 113 - DNA, 369 bp, synthetic construct
  source 1..369: mol_type = other DNA
caggtggtattggtggagtctgggggaggcgtggtccagcctgggaggtccctgagactctcctgtgcagcgtctggattcaccttcagtagctatggcatgcactgggtccgccaggctccaggcaaggggctggagtgggtggcagttatttggtatgatggaagtattaaatactatgcagactccgtgaagggccgattcaccatctccagagacaattccaataacacaatctatctgcaaatgaacagcctgagagccgaggacacggctgtatattactgtgcgagagcccttataccagtggctggtacggaccccatttttgggtactggggccagggaaccctggtcaccgtctcctca

SEQ ID NO: 114 - protein, 123 aa, synthetic construct
QVVLVESGGGVVQPGRSLRLSCAASGFTFSSYGMHWVRQAPGKGLEWVAVIWYDGSIKYYADSVKGRFTISRDNSNNTIYLQMNSLRAEDTAVYYCARALIPVAGTDPIFGYWGQGTLVTVSS

SEQ ID NO: 115 - DNA, 24 bp, synthetic construct
  source 1..24: mol_type = other DNA
ggattcaccttcagtagctatggc

SEQ ID NO: 116 - protein, 8 aa, synthetic construct
GFTFSSYG

SEQ ID NO: 117 - DNA, 24 bp, synthetic construct
  source 1..24: mol_type = other DNA
atttggtatgatggaagtattaaa

SEQ ID NO: 118 - protein, 8 aa, synthetic construct
IWYDGSIK

SEQ ID NO: 119 - DNA, 48 bp, synthetic construct
  source 1..48: mol_type = other DNA
gcgagagcccttataccagtggctggtacggaccccatttttgggtac

SEQ ID NO: 120 - protein, 16 aa, synthetic construct
ARALIPVAGTDPIFGY

SEQ ID NO: 121 - DNA, 321 bp, synthetic construct
  source 1..321: mol_type = other DNA
gacatccagatgacccagtctccttccaccctgtctgcatctgtaggagacagagtcaccatcacttgccgggccagtcagagtgttagtagctggttggcctggtatcagcagaaaccagggaaagcccctaaactcctgatctttaagacgtctggtttagaaagtggggtcccatttaggttcagcggcagtggatctgggacagaattcactctcaccatcagcagcctgcagcctgatgattttgcaacttattactgccaacagtataatacttattctccgacgttcggccaagggaccaaggtggagatcaaa

SEQ ID NO: 122 - protein, 107 aa, synthetic construct
DIQMTQSPSTLSASVGDRVTITCRASQSVSSWLAWYQQKPGKAPKLLIFKTSGLESGVPFRFSGSGSGTEFTLTISSLQPDDFATYYCQQYNTYSPTFGQGTKVEIK

SEQ ID NO: 123 - DNA, 18 bp, synthetic construct
  source 1..18: mol_type = other DNA
cagagtgttagtagctgg

SEQ ID NO: 124 - protein, 6 aa, synthetic construct
QSVSSW

SEQ ID NO: 125-126 - skipped (2 entries)

SEQ ID NO: 127 - DNA, 27 bp, synthetic construct
  source 1..27: mol_type = other DNA
caacagtataatacttattctccgacg

SEQ ID NO: 128 - protein, 9 aa, synthetic construct
QQYNTYSPT

SEQ ID NO: 129 - DNA, 360 bp, synthetic construct
  source 1..360: mol_type = other DNA
caggtgcagctgcaggagtcgggcccaggactggtgaagccttcggagaccctgtccctcacctgcactgtctctggtggctccatcagtagttactactggagctggatccggcagcccccagggaagggactggagtggattggatatatctattacagtgggagaaccaactacaacccctccctcaagagtcgagtcaccatatcagtggacacgtccaagaaccagttctccctgaaactgagctctgtgaccgccgcagacacggccatttattactgtgcgagacatcgtgtaactagaactgcggactcctttgactactggggccagggaaccctggtcaccgtctcctca

SEQ ID NO: 130 - protein, 120 aa, synthetic construct
QVQLQESGPGLVKPSETLSLTCTVSGGSISSYYWSWIRQPPGKGLEWIGYIYYSGRTNYNPSLKSRVTISVDTSKNQFSLKLSSVTAADTAIYYCARHRVTRTADSFDYWGQGTLVTVSS

SEQ ID NO: 131 - DNA, 24 bp, synthetic construct
  source 1..24: mol_type = other DNA
ggtggctccatcagtagttactac

SEQ ID NO: 132 - protein, 8 aa, synthetic construct
GGSISSYY

SEQ ID NO: 133 - DNA, 21 bp, synthetic construct
  source 1..21: mol_type = other DNA
atctattacagtgggagaacc

SEQ ID NO: 134 - protein, 7 aa, synthetic construct
IYYSGRT

SEQ ID NO: 135 - DNA, 42 bp, synthetic construct
  source 1..42: mol_type = other DNA
gcgagacatcgtgtaactagaactgcggactcctttgactac

SEQ ID NO: 136 - protein, 14 aa, synthetic construct
ARHRVTRTADSFDY

SEQ ID NO: 137 - DNA, 321 bp, synthetic construct
  source 1..321: mol_type = other DNA
gacatccagatgacccagtctccatcctccctgtctgcatctgtaggagacagagtcaccatcacttgccaggcgagtcaggacattaacaactatttaaattggtatcagcagaaaacagggaaagcccctaagttcctgatctacgatgcatccaatttggaaacaggggtctcatcaaggttcagtggaagtggatctgggacagattttactttcaccatcagcagcctgcagcctgaagatgttggaacatattactgtcaccagtatggtgatctcccgtacacttttggccaggggaccaagctggagatcaaa

SEQ ID NO: 138 - protein, 107 aa, synthetic construct
DIQMTQSPSSLSASVGDRVTITCQASQDINNYLNWYQQKTGKAPKFLIYDASNLETGVSSRFSGSGSGTDFTFTISSLQPEDVGTYYCHQYGDLPYTFGQGTKLEIK

SEQ ID NO: 139 - DNA, 18 bp, synthetic construct
  source 1..18: mol_type = other DNA
caggacattaacaactat

SEQ ID NO: 140 - protein, 6 aa, synthetic construct
QDINNY

SEQ ID NO: 141-142 - skipped (2 entries)

SEQ ID NO: 143 - DNA, 27 bp, synthetic construct
  source 1..27: mol_type = other DNA
caccagtatggtgatctcccgtacact

SEQ ID NO: 144 - protein, 9 aa, synthetic construct
HQYGDLPYT

SEQ ID NO: 145 - DNA, 360 bp, synthetic construct
  source 1..360: mol_type = other DNA
gaggtgcagctggtggagtctgggggaggcctggtcaggccggggggatccctgagactctcctgtgcagcctctggattcaccttcactagctatgccatgaattgggtccgccaggctccagggaagggactggagtgggtctcatccatttctagttatagttcttacatatatatcgcagactcagtgaagggccgattcaccctctccagagacaatgccaagaactcactgtatctacaaatgcacagtctgagacccgaggacacggctgtttatttctgtgcgagagagggatatagtgcctactcctactttgacttctggggccggggaaccctggtcaccgtctcctca

SEQ ID NO: 146 - protein, 120 aa, synthetic construct
EVQLVESGGGLVRPGGSLRLSCAASGFTFTSYAMNWVRQAPGKGLEWVSSISSYSSYIYIADSVKGRFTLSRDNAKNSLYLQMHSLRPEDTAVYFCAREGYSAYSYFDFWGRGTLVTVSS

SEQ ID NO: 147 - DNA, 24 bp, synthetic construct
  source 1..24: mol_type = other DNA
ggattcaccttcactagctatgcc

SEQ ID NO: 148 - protein, 8 aa, synthetic construct
GFTFTSYA

SEQ ID NO: 149 - DNA, 24 bp, synthetic construct
  source 1..24: mol_type = other DNA
atttctagttatagttcttacata

SEQ ID NO: 150 - protein, 8 aa, synthetic construct
ISSYSSYI

SEQ ID NO: 151 - DNA, 39 bp, synthetic construct
  source 1..39: mol_type = other DNA
gcgagagagggatatagtgcctactcctactttgacttc

SEQ ID NO: 152 - protein, 13 aa, synthetic construct
AREGYSAYSYFDF

SEQ ID NO: 153 - DNA, 321 bp, synthetic construct
  source 1..321: mol_type = other DNA
gacatccagatgacccagtctccttccaccctgtctgcatctgttggagacagagtcaccatcacttgccgggccagtcagagtgttattagttggttggcctggtatcaacagaaaccagggaaagcccctaaactcctgatccatagggcgtctagtttagaaagtggggtcccatcaaggttcagcggcagtggatctgggacagaattcactctcaccatcagcggcctgcagcctgatgattttgcaacttattactgtcaacagtataatacttattttccgacgttcggccaagggaccaaggtggaagtcaaa

SEQ ID NO: 154 - protein, 107 aa, synthetic construct
DIQMTQSPSTLSASVGDRVTITCRASQSVISWLAWYQQKPGKAPKLLIHRASSLESGVPSRFSGSGSGTEFTLTISGLQPDDFATYYCQQYNTYFPTFGQGTKVEVK

SEQ ID NO: 155 - DNA, 18 bp, synthetic construct
  source 1..18: mol_type = other DNA
cagagtgttattagttgg

SEQ ID NO: 156 - protein, 6 aa, synthetic construct
QSVISW

SEQ ID NO: 157-158 - skipped (2 entries)

SEQ ID NO: 159 - DNA, 27 bp, synthetic construct
  source 1..27: mol_type = other DNA
caacagtataatacttattttccgacg

SEQ ID NO: 160 - protein, 9 aa, synthetic construct
QQYNTYFPT

SEQ ID NO: 161 - DNA, 354 bp, synthetic construct
  source 1..354: mol_type = other DNA
gaggtggaactgttggaatctgggggaggcttggtacagcctggggggtccctgagactctcctgtgcagcctctggattcacctttagtagttatgccatgagttgggtccgccaggctccagggaaggggctggagtgggtctcatctattagtggtagggttggtagcacacatttcgcagactccgtgaagggccggttcaccttctccagagacaattccaagaacacgctgtatctgcagctgagcagcctgagagccgaggacacggccgtatattactgtgcgagaagtagaggagcagcctactttgactactggggccagggaaccctggtcaccgtctcctca

SEQ ID NO: 162 - protein, 118 aa, synthetic construct
EVELLESGGGLVQPGGSLRLSCAASGFTFSSYAMSWVRQAPGKGLEWVSSISGRVGSTHFADSVKGRFTFSRDNSKNTLYLQLSSLRAEDTAVYYCARSRGAAYFDYWGQGTLVTVSS

SEQ ID NO: 163 - DNA, 24 bp, synthetic construct
  source 1..24: mol_type = other DNA
ggattcacctttagtagttatgcc

SEQ ID NO: 164 - protein, 8 aa, synthetic construct
GFTFSSYA

SEQ ID NO: 165 - DNA, 24 bp, synthetic construct
  source 1..24: mol_type = other DNA
attagtggtagggttggtagcaca

SEQ ID NO: 166 - protein, 8 aa, synthetic construct
ISGRVGST

SEQ ID NO: 167 - DNA, 33 bp, synthetic construct
  source 1..33: mol_type = other DNA
gcgagaagtagaggagcagcctactttgactac

SEQ ID NO: 168 - protein, 11 aa, synthetic construct
ARSRGAAYFDY

SEQ ID NO: 169 - DNA, 321 bp, synthetic construct
  source 1..321: mol_type = other DNA
gacatccagatgacccagtctccatcctccctgtctgcatctgtaggagacagagtcaccatcacttgccaggcgaatcaggacattagcaactttttaaattggtatcagcagagaccagggaaagcccctaacctcctgatctatgctgcatccaatttggaaacaggggtcccatcaaggttcagtggaagtggatctgggacagattttactttcaccatcagcagcctgcagcctgaagatattgcaacatattactgtcaacattatgataattttccattcactttcggccctgggaccaaggtggatatcaaa

SEQ ID NO: 170 - protein, 107 aa, synthetic construct
DIQMTQSPSSLSASVGDRVTITCQANQDISNFLNWYQQRPGKAPNLLIYAASNLETGVPSRFSGSGSGTDFTFTISSLQPEDIATYYCQHYDNFPFTFGPGTKVDIK

SEQ ID NO: 171 - DNA, 18 bp, synthetic construct
  source 1..18: mol_type = other DNA
caggacattagcaacttt

SEQ ID NO: 172 - protein, 6 aa, synthetic construct
QDISNF

SEQ ID NO: 173-174 - skipped (2 entries)

SEQ ID NO: 175 - DNA, 27 bp, synthetic construct
  source 1..27: mol_type = other DNA
caacattatgataattttccattcact

SEQ ID NO: 176 - protein, 9 aa, synthetic construct
QHYDNFPFT

SEQ ID NO: 177 - DNA, 363 bp, synthetic construct
  source 1..363: mol_type = other DNA
cagctgcagctgcaggagtcgggcccaggactggtgaacccttcggagaccctgtccctcacctgctctgtctctggtggctccatcagcagtgttaattactactggggctggatccgccagtccccagggaagggactggagtggattgggagtatctattatactgggagtaccgactacaacccgtccctcaagaatcgagtcaccatatccgtagacacgtccaagaaccagttctccctgaagcagacttctgtgaccgccgcagacacggctgtctattactgtgcgagacatgtggcactggctgggggggcttttgatatctggggccaggggacaatggtcaccgtctcttca

SEQ ID NO: 178 - protein, 121 aa, synthetic construct
QLQLQESGPGLVNPSETLSLTCSVSGGSISSVNYYWGWIRQSPGKGLEWIGSIYYTGSTDYNPSLKNRVTISVDTSKNQFSLKQTSVTAADTAVYYCARHVALAGGAFDIWGQGTMVTVSS

SEQ ID NO: 179 - DNA, 30 bp, synthetic construct
  source 1..30: mol_type = other DNA
ggtggctccatcagcagtgttaattactac

SEQ ID NO: 180 - protein, 10 aa, synthetic construct
GGSISSVNYY

SEQ ID NO: 181 - DNA, 21 bp, synthetic construct
  source 1..21: mol_type = other DNA
atctattatactgggagtacc

SEQ ID NO: 182 - protein, 7 aa, synthetic construct
IYYTGST

SEQ ID NO: 183 - DNA, 39 bp, synthetic construct
  source 1..39: mol_type = other DNA
gcgagacatgtggcactggctgggggggcttttgatatc

SEQ ID NO: 184 - protein, 13 aa, synthetic construct
ARHVALAGGAFDI

SEQ ID NO: 185 - DNA, 315 bp, synthetic construct
  source 1..315: mol_type = other DNA
gaaattgtgttgacgcagtctccaggcaccctgtctttgtctccaggggaaagagccaccctctcctgcagggccagtcagagtgttagtagcagcttcttaggctggtaccaacagaaacctggccaggctcccaggctcctcatctatggttcttccaccagggccactggcatcccagacaggttcagtggcagtgggtctgggacagacttcaatatcaccatcagcagactggagcctgaagattttgcagtgtattactgtcagcagtttggtaggtccttcggccctgggaccaagctggagatcaaa

SEQ ID NO: 186 - protein, 105 aa, synthetic construct
EIVLTQSPGTLSLSPGERATLSCRASQSVSSSFLGWYQQKPGQAPRLLIYGSSTRATGIPDRFSGSGSGTDFNITISRLEPEDFAVYYCQQFGRSFGPGTKLEIK

SEQ ID NO: 187 - DNA, 21 bp, synthetic construct
  source 1..21: mol_type = other DNA
cagagtgttagtagcagcttc

SEQ ID NO: 188 - protein, 7 aa, synthetic construct
QSVSSSF

SEQ ID NO: 189-190 - skipped (2 entries)

SEQ ID NO: 191 - DNA, 18 bp, synthetic construct
  source 1..18: mol_type = other DNA
cagcagtttggtaggtcc

SEQ ID NO: 192 - protein, 6 aa, synthetic construct
QQFGRS

SEQ ID NO: 193 - DNA, 360 bp, synthetic construct
  source 1..360: mol_type = other DNA
caggtgcagctggtggagtctgggggaggcttggtcaagcctggagggtccctgagactctcctgcgcagcctctggattcaccttcagtgactattacatgaactggatccgccaggctccagggaaggggctggagtggatttcatatattagtagtggtggtagtaccacatactacgcagactctgtgaagggccgattcaccatctccagggacaacgccaagaactcactgtatctgcaaatgaacagcctgagagccgaggacacggccgtgtattactgtgcgagagatgggaagtacaacacctcgccgggggactactggggccagggaaccctggtcaccgtctcctca

SEQ ID NO: 194 - protein, 120 aa, synthetic construct
QVQLVESGGGLVKPGGSLRLSCAASGFTFSDYYMNWIRQAPGKGLEWISYISSGGSTTYYADSVKGRFTISRDNAKNSLYLQMNSLRAEDTAVYYCARDGKYNTSPGDYWGQGTLVTVSS

SEQ ID NO: 195 - DNA, 24 bp, synthetic construct
  source 1..24: mol_type = other DNA
ggattcaccttcagtgactattac

SEQ ID NO: 196 - protein, 8 aa, synthetic construct
GFTFSDYY

SEQ ID NO: 197 - DNA, 24 bp, synthetic construct
  source 1..24: mol_type = other DNA
attagtagtggtggtagtaccaca

SEQ ID NO: 198 - protein, 8 aa, synthetic construct
ISSGGSTT

SEQ ID NO: 199 - DNA, 39 bp, synthetic construct
  source 1..39: mol_type = other DNA
gcgagagatgggaagtacaacacctcgccgggggactac

SEQ ID NO: 200 - protein, 13 aa, synthetic construct
ARDGKYNTSPGDY

SEQ ID NO: 201 - DNA, 318 bp, synthetic construct
  source 1..318: mol_type = other DNA
gaaattgtgttgacgcagtctccagactttcagtctgtgactccaaaggagaaagtcaccatcacctgccgggccagtcagtccattggtagtagcttacactggtaccagcagaaaccagatcagtctccaaagctcctcatcaagtatgcttcccagtccttctcaggggtcccctcgaggttcagtggcagtggatctgggacagatttcaccctcaccatcaatagcctggaagctgaagatgctgctacgtattactgtcttcagagtagtagtttacggacgttcggccaagggaccaaagtggatatcaaa

SEQ ID NO: 202 - protein, 106 aa, synthetic construct
EIVLTQSPDFQSVTPKEKVTITCRASQSIGSSLHWYQQKPDQSPKLLIKYASQSFSGVPSRFSGSGSGTDFTLTINSLEAEDAATYYCLQSSSLRTFGQGTKVDIK

SEQ ID NO: 203 - DNA, 18 bp, synthetic construct
  source 1..18: mol_type = other DNA
cagtccattggtagtagc

SEQ ID NO: 204 - protein, 6 aa, synthetic construct
QSIGSS

SEQ ID NO: 205-206 - skipped (2 entries)

SEQ ID NO: 207 - DNA, 24 bp, synthetic construct
  source 1..24: mol_type = other DNA
cttcagagtagtagtttacggacg

SEQ ID NO: 208 - protein, 8 aa, synthetic construct
LQSSSLRT

SEQ ID NO: 209 - DNA, 360 bp, synthetic construct
  source 1..360: mol_type = other DNA
caggtgcagctggtggagtctgggggaggcttggtcaagcctggagggtccctgagactctcctgtgcagcctctggattcaccttcagtgactactacatgaactggatccgccaggctccagggaaggggctggagtggatttcatacattagtagtagtggtagtaccacatactacgcagactctgtgaagggccgattcaccatctccagggacaacgccaagaactcactgtttctgcaaatgaacagcctgagaggcgaggacacggccgtgtattattgtgcgagagatgggagatacaacaccgtcgccggggactactggggccagggaaccacggtcaccgtctcctca

SEQ ID NO: 210 - protein, 120 aa, synthetic construct
QVQLVESGGGLVKPGGSLRLSCAASGFTFSDYYMNWIRQAPGKGLEWISYISSSGSTTYYADSVKGRFTISRDNAKNSLFLQMNSLRGEDTAVYYCARDGRYNTVAGDYWGQGTTVTVSS

SEQ ID NO: 211 - DNA, 24 bp, synthetic construct
  source 1..24: mol_type = other DNA
ggattcaccttcagtgactactac

SEQ ID NO: 212 - protein, 8 aa, synthetic construct
GFTFSDYY

SEQ ID NO: 213 - DNA, 24 bp, synthetic construct
  source 1..24: mol_type = other DNA
attagtagtagtggtagtaccaca

SEQ ID NO: 214 - protein, 8 aa, synthetic construct
ISSSGSTT

SEQ ID NO: 215 - DNA, 39 bp, synthetic construct
  source 1..39: mol_type = other DNA
gcgagagatgggagatacaacaccgtcgccggggactac

SEQ ID NO: 216 - protein, 13 aa, synthetic construct
ARDGRYNTVAGDY

SEQ ID NO: 217 - DNA, 321 bp, synthetic construct
  source 1..321: mol_type = other DNA
gaaattgtgatgacgcagtctccagactttcagtctgtggctccaaaggagaaagtcaccatcacctgccgggccagtcagaacattggtggtagcttacactggtaccagcagaaaccagatcagtctccaaagctcctcatcaagtatgcttcccagtccttctcaggggtcccctcgaggttcagtggcagtggatctgggacagatttcaccctcaccatcaatagcctggaagctgaagatgctgcaacgtattactgtcttcagagttacactttacggacgttcggccaagggaccaaggtggagatcaaacga

SEQ ID NO: 218 - protein, 107 aa, synthetic construct
EIVMTQSPDFQSVAPKEKVTITCRASQNIGGSLHWYQQKPDQSPKLLIKYASQSFSGVPSRFSGSGSGTDFTLTINSLEAEDAATYYCLQSYTLRTFGQGTKVEIKR

SEQ ID NO: 219 - DNA, 18 bp, synthetic construct
  source 1..18: mol_type = other DNA
cagaacattggtggtagc

SEQ ID NO: 220 - protein, 6 aa, synthetic construct
QNIGGS

SEQ ID NO: 221-222 - skipped (2 entries)

SEQ ID NO: 223 - DNA, 24 bp, synthetic construct
  source 1..24: mol_type = other DNA
cttcagagttacactttacggacg

SEQ ID NO: 224 - protein, 8 aa, synthetic construct
LQSYTLRT

SEQ ID NO: 225 - DNA, 360 bp, synthetic construct
  source 1..360: mol_type = other DNA
gaggtgcagctggtggagtctgggggaggcttggtcaagcctggagggtccctgagactctcctgtgcagcctctggattcaccttcagtgactactacatgaactggatccgccaggctccagggaaggggctggagtgggtttcatatattagtagtagtggcagcagtatttattacgcagactctgtgaagggccgattcaccatctccagggacaacgccaagaactcactgtatctgctaatgaacagcctgagagccgaggacacggccgtgtattactgtgcgagagatgggaagtataacagttcgccgggggactactggggccagggaaccctggtcaccgtctcctca

SEQ ID NO: 226 - protein, 120 aa, synthetic construct
EVQLVESGGGLVKPGGSLRLSCAASGFTFSDYYMNWIRQAPGKGLEWVSYISSSGSSIYYADSVKGRFTISRDNAKNSLYLLMNSLRAEDTAVYYCARDGKYNSSPGDYWGQGTLVTVSS

SEQ ID NO: 227 - DNA, 24 bp, synthetic construct
  source 1..24: mol_type = other DNA
ggattcaccttcagtgactactac

SEQ ID NO: 228 - protein, 8 aa, synthetic construct
GFTFSDYY

SEQ ID NO: 229 - DNA, 24 bp, synthetic construct
  source 1..24: mol_type = other DNA
attagtagtagtggcagcagtatt

SEQ ID NO: 230 - protein, 8 aa, synthetic construct
ISSSGSSI

SEQ ID NO: 231 - DNA, 39 bp, synthetic construct
  source 1..39: mol_type = other DNA
gcgagagatgggaagtataacagttcgccgggggactac

SEQ ID NO: 232 - protein, 13 aa, synthetic construct
ARDGKYNSSPGDY

SEQ ID NO: 233 - DNA, 324 bp, synthetic construct
  source 1..324: mol_type = other DNA
gacatccagatgacccagtctccatcctcactgtctgcatctgtaggagacagagtcaccatcgcttgtcgggcgagtcagggcattagcaattatttagcctggtttcagcagaaaccagggaaagcccctaagtccctgatctatgctgcatccagtttgcaaagtggggtcccatcaaagttcagcggcagtggatctgggacagatttcactctcaccatcagcagcctgcagcctgaagattttgcaacttattactgccaacagtataacagttacccgctcactttcggcggagggaccaaggtggaaatcaaacga

SEQ ID NO: 234 - protein, 108 aa, synthetic construct
DIQMTQSPSSLSASVGDRVTIACRASQGISNYLAWFQQKPGKAPKSLIYAASSLQSGVPSKFSGSGSGTDFTLTISSLQPEDFATYYCQQYNSYPLTFGGGTKVEIKR

SEQ ID NO: 235 - DNA, 18 bp, synthetic construct
  source 1..18: mol_type = other DNA
cagggcattagcaattat

SEQ ID NO: 236 - protein, 6 aa, synthetic construct
QGISNY

SEQ ID NO: 237-238 - skipped (2 entries)

SEQ ID NO: 239 - DNA, 27 bp, synthetic construct
  source 1..27: mol_type = other DNA
caacagtataacagttacccgctcact

SEQ ID NO: 240 - protein, 9 aa, synthetic construct
QQYNSYPLT

SEQ ID NO: 241 - DNA, 360 bp, synthetic construct
  source 1..360: mol_type = other DNA
gaggtgcagctggtggagtctgggggaggcttggtcaagcctggagggtccctgagactctcctgtgcagcctctggattcaccttcagtgactactacatgaactggatccgccaggctccagggaaggggctggagtgggtttcatatattagtagtagtggcagtagtatttattacgcagactctgtgaagggccgattcaccatctccagggacaacgccaagaactcactgtatctgctaatgaacagcctgagagccgaggacacggccgtgtattactgtgcgagagatgggaagtataacagctcgccgggggactactggggccagggaaccctggtcactgtctcctca

SEQ ID NO: 242 - protein, 120 aa, synthetic construct; identical to SEQ ID NO: 226

SEQ ID NO: 243 - DNA, 24 bp, synthetic construct
  source 1..24: mol_type = other DNA
ggattcaccttcagtgactactac

SEQ ID NO: 244 - protein, 8 aa, synthetic construct
GFTFSDYY

SEQ ID NO: 245 - DNA, 24 bp, synthetic construct
  source 1..24: mol_type = other DNA
attagtagtagtggcagtagtatt

SEQ ID NO: 246 - protein, 8 aa, synthetic construct
ISSSGSSI

SEQ ID NO: 247 - DNA, 39 bp, synthetic construct
  source 1..39: mol_type = other DNA
gcgagagatgggaagtataacagctcgccgggggactac

SEQ ID NO: 248 - protein, 13 aa, synthetic construct
ARDGKYNSSPGDY

SEQ ID NO: 249 - DNA, 318 bp, synthetic construct
  source 1..318: mol_type = other DNA
gaaattgtgctgactcagtctccagactttcagtctgtgactccaaaggagaaagtcaccatcacctgccgggccagtcagagcattggtggtagcttacactggtaccagcagaaaccagatcagtctccaaagctcctcatcaagtatgcttcccagtccttctcaggggtcccctcgaggttcagtggcagtggatctgggacagatttcaccctcaccatcaatagcctggaagctgaagatgctgcaacgtattactgtcttcagagtagtagtttacggacgttcggccaagggaccaaggtggaaatcaaa

SEQ ID NO: 250 - protein, 106 aa, synthetic construct
EIVLTQSPDFQSVTPKEKVTITCRASQSIGGSLHWYQQKPDQSPKLLIKYASQSFSGVPSRFSGSGSGTDFTLTINSLEAEDAATYYCLQSSSLRTFGQGTKVEIK

SEQ ID NO: 251 - DNA, 18 bp, synthetic construct
  source 1..18: mol_type = other DNA
cagagcattggtggtagc

SEQ ID NO: 252 - protein, 6 aa, synthetic construct
QSIGGS

SEQ ID NO: 253-254 - skipped (2 entries)

SEQ ID NO: 255 - DNA, 24 bp, synthetic construct
  source 1..24: mol_type = other DNA
cttcagagtagtagtttacggacg

SEQ ID NO: 256 - protein, 8 aa, synthetic construct
LQSSSLRT

SEQ ID NO: 257 - DNA, 363 bp, synthetic construct
  source 1..363: mol_type = other DNA
gaggtgcagctggtggagtctgggggaggcttggtcaagcctggagggtccctgagactctcctgtgcagcctctggattcaccttcagtgactactacatgaactggatccgccaggctccagggaaggggctggagtgggtttcatacattagtagtagtggtaattccatatactacgcagactctgtgaagggccgattcaccatctccagggacaacgccaagaactcactgtttctgcagatgagcagcctgagagccgaggacacggccgtgtattactgtgcgagagatgggaggtataacgaccgtcgccgggggtactactggggccagggaaccctggtcaccgtctcctca

SEQ ID NO: 258 - protein, 121 aa, synthetic construct
EVQLVESGGGLVKPGGSLRLSCAASGFTFSDYYMNWIRQAPGKGLEWVSYISSSGNSIYYADSVKGRFTISRDNAKNSLFLQMSSLRAEDTAVYYCARDGRYNDRRRGYYWGQGTLVTVSS

SEQ ID NO: 259 - DNA, 24 bp, synthetic construct
  source 1..24: mol_type = other DNA
ggattcaccttcagtgactactac

SEQ ID NO: 260 - protein, 8 aa, synthetic construct
GFTFSDYY

SEQ ID NO: 261 - DNA, 24 bp, synthetic construct
  source 1..24: mol_type = other DNA
attagtagtagtggtaattccata

SEQ ID NO: 262 - protein, 8 aa, synthetic construct
ISSSGNSI

SEQ ID NO: 263 - DNA, 42 bp, synthetic construct
  source 1..42: mol_type = other DNA
gcgagagatgggaggtataacgaccgtcgccgggggtactac

SEQ ID NO: 264 - protein, 14 aa, synthetic construct
ARDGRYNDRRRGYY

SEQ ID NO: 265 - DNA, 321 bp, synthetic construct
  source 1..321: mol_type = other DNA
gaaattgtgatgacgcagtctccagactttcagtctgtgactccaaaggagaaagtcaccatcacctgccgggccagtcagagcattggtagtagcttacactggtaccagcagaaaccagatcagtctccaaaactcctcatcaagtatgcttcccagtccttctcaggggtcccctcgaggttcagtggcagtggatctgggacagatttcaccctcaccatcaatagcctggaagctgaagatgctgcaacgtattactgtcttcagagtagtagtttacggacgttcggccaagggaccaaggtggagatcaaacga

SEQ ID NO: 266 - protein, 107 aa, synthetic construct
EIVMTQSPDFQSVTPKEKVTITCRASQSIGSSLHWYQQKPDQSPKLLIKYASQSFSGVPSRFSGSGSGTDFTLTINSLEAEDAATYYCLQSSSLRTFGQGTKVEIKR

SEQ ID NO: 267 - DNA, 18 bp, synthetic construct
  source 1..18: mol_type = other DNA
cagagcattggtagtagc

SEQ ID NO: 268 - protein, 6 aa, synthetic construct
QSIGSS

SEQ ID NO: 269-270 - skipped (2 entries)

SEQ ID NO: 271 - DNA, 24 bp, synthetic construct
  source 1..24: mol_type = other DNA
cttcagagtagtagtttacggacg

SEQ ID NO: 272 - protein, 8 aa, synthetic construct
LQSSSLRT

SEQ ID NO: 273 - DNA, 360 bp, synthetic construct
  source 1..360: mol_type = other DNA
gaggtgcagctggtggagtctgggggaggcttggtcaagcctggagggtccctgagactctcctgtgcagcctctggattcaccttcagtgactactacatgaactggatccgccaggctccagggaaggggctggagtggatttcatatcttagtagtagtggtagtgccacatactacgcagactctgtgaagggccgattcaccatctccagggacaacgccaagaactcactgtatctgcaaatgaacagcctgagagccgaggacacggccgtgtattattgtgcgagagatgggaagtacaacaccgtcgccggggactactggggccagggaaccacggtcaccgtctcctca

SEQ ID NO: 274 - protein, 120 aa, synthetic construct
EVQLVESGGGLVKPGGSLRLSCAASGFTFSDYYMNWIRQAPGKGLEWISYLSSSGSATYYADSVKGRFTISRDNAKNSLYLQMNSLRAEDTAVYYCARDGKYNTVAGDYWGQGTTVTVSS

SEQ ID NO: 275 - DNA, 24 bp, synthetic construct
  source 1..24: mol_type = other DNA
ggattcaccttcagtgactactac

SEQ ID NO: 276 - protein, 8 aa, synthetic construct
GFTFSDYY

SEQ ID NO: 277 - DNA, 24 bp, synthetic construct
  source 1..24: mol_type = other DNA
cttagtagtagtggtagtgccaca

SEQ ID NO: 278 - protein, 8 aa, synthetic construct
LSSSGSAT

SEQ ID NO: 279 - DNA, 39 bp, synthetic construct
  source 1..39: mol_type = other DNA
gcgagagatgggaagtacaacaccgtcgccggggactac

SEQ ID NO: 280 - protein, 13 aa, synthetic construct
ARDGKYNTVAGDY

SEQ ID NO: 281 - DNA, 324 bp, synthetic construct
  source 1..324: mol_type = other DNA
gacatccagttgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttacaaagtggggtcccatcaaggttcagcggcagtggatctgggacagaattcactctcacaatcagcagcctgcagcctgaagattttgcaacttattactgtctacaacataatagttacccgtacacttttggccaggggaccaaggtagagatcaaacga

SEQ ID NO: 282 - protein, 108 aa, synthetic construct
DIQLTQSPSSLSASVGDRVTITCRASQGIRNDLGWYQQKPGKAPKRLIYAASSLQSGVPSRFSGSGSGTEFTLTISSLQPEDFATYYCLQHNSYPYTFGQGTKVEIKR

SEQ ID NO: 283 - DNA, 18 bp, synthetic construct
  source 1..18: mol_type = other DNA
cagggcattagaaatgat

SEQ ID NO: 284 - protein, 6 aa, synthetic construct
QGIRND

SEQ ID NO: 285-286 - skipped (2 entries)

SEQ ID NO: 287 - DNA, 27 bp, synthetic construct
  source 1..27: mol_type = other DNA
ctacaacataatagttacccgtacact

SEQ ID NO: 288 - protein, 9 aa, synthetic construct
LQHNSYPYT

SEQ ID NO: 289 - DNA, 360 bp, synthetic construct
  source 1..360: mol_type = other DNA
gaggtgcagctggtggagtctgggggaggcttggtcaagcctggagggtccctgagactctcctgtgcagcctctggattcaccttcagtgactactacatgaactggatccgccaggctccagggaaggggctggagtggatttcatacattagtagtagtgatgataccacatactacgcagactctgtgaagggccgattcaccatatccagggacaacgccaagaactcactgtatctgcaaatgaacagcctgagagccgaggacacggccgtgtattattgtgcgagagatgggaagtacaacaccgtcgccggggaccactggggccagggaaccctggtcaccgtctcctca

SEQ ID NO: 290 - protein, 120 aa, synthetic construct
EVQLVESGGGLVKPGGSLRLSCAASGFTFSDYYMNWIRQAPGKGLEWISYISSSDDTTYYADSVKGRFTISRDNAKNSLYLQMNSLRAEDTAVYYCARDGKYNTVAGDHWGQGTLVTVSS

SEQ ID NO: 291 - DNA, 24 bp, synthetic construct
  source 1..24: mol_type = other DNA
ggattcaccttcagtgactactac

SEQ ID NO: 292 - protein, 8 aa, synthetic construct
GFTFSDYY

SEQ ID NO: 293 - DNA, 24 bp, synthetic construct
  source 1..24: mol_type = other DNA
attagtagtagtgatgataccaca

SEQ ID NO: 294 - protein, 8 aa, synthetic construct
ISSSDDTT

SEQ ID NO: 295 - DNA, 39 bp, synthetic construct
  source 1..39: mol_type = other DNA
gcgagagatgggaagtacaacaccgtcgccggggaccac

SEQ ID NO: 296 - protein, 13 aa, synthetic construct
ARDGKYNTVAGDH

SEQ ID NO: 297 - DNA, 321 bp, synthetic construct
  source 1..321: mol_type = other DNA
gaaattgtgatgacacagtctccagactttcagtctgtggctccaaaggagaaagtcaccatcacctgccgggccagtcagaacattggtagtagcttacactggtaccagcagaaaccagatcagtctccaaagctcctcatcaagtatgcttcccagtccttctcaggggtcccctcgaggttcagtggcagtggatctgggacagatttcaccctcaccatcaatagcctggaagctgaagatgctgcaacgtattactgtcttcagagttatactttaaggacgttcggccaagggaccaaggtggagatcaaacga

SEQ ID NO: 298 - protein, 107 aa, synthetic construct
EIVMTQSPDFQSVAPKEKVTITCRASQNIGSSLHWYQQKPDQSPKLLIKYASQSFSGVPSRFSGSGSGTDFTLTINSLEAEDAATYYCLQSYTLRTFGQGTKVEIKR

SEQ ID NO: 299 - DNA, 18 bp, synthetic construct
  source 1..18: mol_type = other DNA
cagaacattggtagtagc

SEQ ID NO: 300 - protein, 6 aa, synthetic construct
QNIGSS

SEQ ID NO: 301-302 - skipped (2 entries)

SEQ ID NO: 303 - DNA, 24 bp, synthetic construct
  source 1..24: mol_type = other DNA
cttcagagttatactttaaggacg

SEQ ID NO: 304 - protein, 8 aa, synthetic construct
LQSYTLRT

SEQ ID NO: 305 - DNA, 345 bp, synthetic construct
  source 1..345: mol_type = other DNA
gaggtgcagctggtggagtctgggggaggcttggtacagcctggggggtccctgagactctcctgtgcagcctctggattcacctttagcagttatgccatgagctgggtccgccaggctccagggaaggggctggagtgggtctcagctattagtggtcgtggttataacgcagactacgcagactccgtgaagggccggttcaccatctccagggacaattccaagaacacgctgtatctgcaaatgaacagcctgagagccgaagacacggccgtatattactgtgcgaaattggaatactttgactactggggccagggaaccctggtcactgtctcctca

SEQ ID NO: 306 - protein, 115 aa, synthetic construct
EVQLVESGGGLVQPGGSLRLSCAASGFTFSSYAMSWVRQAPGKGLEWVSAISGRGYNADYADSVKGRFTISRDNSKNTLYLQMNSLRAEDTAVYYCAKLEYFDYWGQGTLVTVSS

SEQ ID NO: 307 - DNA, 24 bp, synthetic construct
  source 1..24: mol_type = other DNA
ggattcacctttagcagttatgcc

SEQ ID NO: 308 - protein, 8 aa, synthetic construct
GFTFSSYA

SEQ ID NO: 309 - DNA, 24 bp, synthetic construct
  source 1..24: mol_type = other DNA
attagtggtcgtggttataacgca

SEQ ID NO: 310 - protein, 8 aa, synthetic construct
ISGRGYNA

SEQ ID NO: 311 - DNA, 24 bp, synthetic construct
  source 1..24: mol_type = other DNA
gcgaaattggaatactttgactac

SEQ ID NO: 312 - protein, 8 aa, synthetic construct
AKLEYFDY

SEQ ID NO: 313 - DNA, 321 bp, synthetic construct
  source 1..321: mol_type = other DNA
gacatccagatgacccagtctccttccaccctgtctgcatctgtaggagacagagtcaccatcacttgccgggccagtcagagtattagtagctggttggcctggtatcagcagaaaccagggaaagcccctaagctcctgatctataaggcgtctagtttagaaagtggggtcccatcaaggttcagcggcagtggatctgggacagatttcactctcaccatcagcagcctgaggcctgaagattttgcaacttattactgccaacagtataatagttaccctctgactttcggcggagggaccaaggtggagatcaaa

SEQ ID NO: 314 - protein, 107 aa, synthetic construct
DIQMTQSPSTLSASVGDRVTITCRASQSISSWLAWYQQKPGKAPKLLIYKASSLESGVPSRFSGSGSGTDFTLTISSLRPEDFATYYCQQYNSYPLTFGGGTKVEIK

SEQ ID NO: 315 - DNA, 18 bp, synthetic construct
  source 1..18: mol_type = other DNA
cagagtattagtagctgg

SEQ ID NO: 316 - protein, 6 aa, synthetic construct
QSISSW

SEQ ID NO: 317-318 - skipped (2 entries)

SEQ ID NO: 319 - DNA, 27 bp, synthetic construct
  source 1..27: mol_type = other DNA
caacagtataatagttaccctctgact

SEQ ID NO: 320 - protein, 9 aa, synthetic construct
QQYNSYPLT

SEQ ID NO: 321 - DNA, 345 bp, synthetic construct
  source 1..345: mol_type = other DNA
gaggtgcagctggtggagtctgggggaggcttggtacagcctggggggtccctgagactctcctgtgcagcctctggattcacctttagcacctatgccatgcactgggtccgccaggctccagggaaggggctggagtgggtctcaagtattagtggtcgtggtcgtaactcagaccacgcagactccgtgaagggccggttcaccatctccagagacaattccaagaacacgctctatctacaaatgaacagcctgagagccgaggacacggccgtatattactgtgcgaggaccgaatacttccaccactggggccagggcaccctggtcaccgtctcctca

SEQ ID NO: 322 - protein, 115 aa, synthetic construct
EVQLVESGGGLVQPGGSLRLSCAASGFTFSTYAMHWVRQAPGKGLEWVSSISGRGRNSDHADSVKGRFTISRDNSKNTLYLQMNSLRAEDTAVYYCARTEYFHHWGQGTLVTVSS

SEQ ID NO: 323 - DNA, 24 bp, synthetic construct
  source 1..24: mol_type = other DNA
ggattcacctttagcacctatgcc

SEQ ID NO: 324 - protein, 8 aa, synthetic construct
GFTFSTYA

SEQ ID NO: 325 - DNA, 24 bp, synthetic construct
  source 1..24: mol_type = other DNA
attagtggtcgtggtcgtaactca

SEQ ID NO: 326 - protein, 8 aa, synthetic construct
ISGRGRNS

SEQ ID NO: 327 - DNA, 24 bp, synthetic construct
  source 1..24: mol_type = other DNA
gcgaggaccgaatacttccaccac

SEQ ID NO: 328 - protein, 8 aa, synthetic construct
ARTEYFHH

SEQ ID NO: 329 - DNA, 321 bp, synthetic construct
  source 1..321: mol_type = other DNA
gacatccagatgacccagtctccatcctcactgtctgcatctgtaggagacagaatcaccatcacttgtcgggcgagtcaggacattaacaattatttagcctggtttcagcagaaaccaggaaaagcccctaagtccctgatctatggtgcatccagcttgcaaagtggggtcccatcaaagttcagcggcagtggatctgggacagatttcactctcaccatcagcagcctgcagcctgaagattttgcaacttattactgccaacaatatagttcttacccattcactttcggccctgggaccaaagtggatatcaaa

SEQ ID NO: 330 - protein, 107 aa, synthetic construct
DIQMTQSPSSLSASVGDRITITCRASQDINNYLAWFQQKPGKAPKSLIYGASSLQSGVPSKFSGSGSGTDFTLTISSLQPEDFATYYCQQYSSYPFTFGPGTKVDIK

SEQ ID NO: 331 - DNA, 18 bp, synthetic construct
  source 1..18: mol_type = other DNA
caggacattaacaattat

SEQ ID NO: 332 - protein, 6 aa, synthetic construct
QDINNY

SEQ ID NO: 333-334 - skipped (2 entries)

SEQ ID NO: 335 - DNA, 27 bp, synthetic construct
  source 1..27: mol_type = other DNA
caacaatatagttcttacccattcact

SEQ ID NO: 336 - protein, 9 aa, synthetic construct
QQYSSYPFT

SEQ ID NO: 337 - DNA, 348 bp, synthetic construct
  source 1..348: mol_type = other DNA
gaggtgcagctggtggagtctgggggaggcttggtccagcctggggggtccctgagactctcctgtgcagcctctggattcatctttagtacctattggatgaactgggtccgccaggctccagggaaggggctggagtgggtggccaacgtgaaccatgatggaagtgaggaatactatgtggactctgtgaagggccgattcaccatctccagagacaacgcccagaattcactgtatctgcaaatggaccgcctgagagccgaggacacggctatgtatttctgtgcgcgaagagccgggctttttgactcctggggccagggaaccctggtcactgtctcctca

SEQ ID NO: 338 - protein, 116 aa, synthetic construct
EVQLVESGGGLVQPGGSLRLSCAASGFIFSTYWMNWVRQAPGKGLEWVANVNHDGSEEYYVDSVKGRFTISRDNAQNSLYLQMDRLRAEDTAMYFCARRAGLFDSWGQGTLVTVSS

SEQ ID NO: 339 - DNA, 24 bp, synthetic construct
  source 1..24: mol_type = other DNA
ggattcatctttagtacctattgg

SEQ ID NO: 340 - protein, 8 aa, synthetic construct
GFIFSTYW

SEQ ID NO: 341 - DNA, 24 bp, synthetic construct
  source 1..24: mol_type = other DNA
gtgaaccatgatggaagtgaggaa

SEQ ID NO: 342 - protein, 8 aa, synthetic construct
VNHDGSEE

SEQ ID NO: 343 - DNA, 27 bp, synthetic construct
  source 1..27: mol_type = other DNA
gcgcgaagagccgggctttttgactcc

SEQ ID NO: 344 - protein, 9 aa, synthetic construct
ARRAGLFDS

SEQ ID NO: 345 - DNA, 321 bp, synthetic construct
  source 1..321: mol_type = other DNA
gacatccagatgacccagtctccatcttccgtgtctgcatctgtaggagacagagtcaccatcacttgtcgggcgagtcaggatattgacaactggttagcctggtatcagcagaaaccagggaaagcccctaaactcctgatctttacttcatccactttgcaaagtggggtcccatcaaggttcagcggcattggatctggaacagatttcactctcaccatcagcagcctacagcctgaagattttgcaacttactattgtcaacaggctaacagtttcccgtggacgttcggccaagggaccaaggtggagatcaaa

SEQ ID NO: 346 - protein, 107 aa, synthetic construct
DIQMTQSPSSVSASVGDRVTITCRASQDIDNWLAWYQQKPGKAPKLLIFTSSTLQSGVPSRFSGIGSGTDFTLTISSLQPEDFATYYCQQANSFPWTFGQGTKVEIK

SEQ ID NO: 347 - DNA, 18 bp, synthetic construct
  source 1..18: mol_type = other DNA
caggatattgacaactgg

SEQ ID NO: 348 - protein, 6 aa, synthetic construct
QDIDNW

SEQ ID NO: 349-350 - skipped (2 entries)

SEQ ID NO: 351 - DNA, 27 bp, synthetic construct
  source 1..27: mol_type = other DNA
caacaggctaacagtttcccgtggacg

SEQ ID NO: 352 - protein, 9 aa, synthetic construct
QQANSFPWT

SEQ ID NO: 353 - DNA, 375 bp, synthetic construct
  source 1..375: mol_type = other DNA
caggtgcagctggtggagtctgggggaggcgtggtcctgcctgggaggtccctgagactctcctgtgcagcgtctggattcacctttagtagttatctcatgtattgggtccgccaggctccaggcaaggggctggagtgggtggcagttatatggtatgatggaagtaataaattctatggagactccgtgaagggccgattcaccatctccagagacaattccaagaacacgttgtatctgcaaatgaacagcctgagagccgaggacacggctgtgtattactgtgcgagagataatgggaatagtggttacgaggacagctggaatgcttttgatatatggggccaagggacaatggtcaccgtctcttca

SEQ ID NO: 354 - protein, 125 aa, synthetic construct
QVQLVESGGGVVLPGRSLRLSCAASGFTFSSYLMYWVRQAPGKGLEWVAVIWYDGSNKFYGDSVKGRFTISRDNSKNTLYLQMNSLRAEDTAVYYCARDNGNSGYEDSWNAFDIWGQGTMVTVSS

SEQ ID NO: 355 - DNA, 24 bp, synthetic construct
  source 1..24: mol_type = other DNA
ggattcacctttagtagttatctc

SEQ ID NO: 356 - protein, 8 aa, synthetic construct
GFTFSSYL

SEQ ID NO: 357 - DNA, 24 bp, synthetic construct
  source 1..24: mol_type = other DNA
atatggtatgatggaagtaataaa

SEQ ID NO: 358 - protein, 8 aa, synthetic construct
IWYDGSNK

SEQ ID NO: 359 - DNA, 54 bp, synthetic construct
  source 1..54: mol_type = other DNA
gcgagagataatgggaatagtggttacgaggacagctggaatgcttttgatata

SEQ ID NO: 360 - protein, 18 aa, synthetic construct
ARDNGNSGYEDSWNAFDI

SEQ ID NO: 361 - DNA, 321 bp, synthetic construct
  source 1..321: mol_type = other DNA
gacatccagatgacccagtctccatcctcactgtctgcatctgtaggagacagagtcaccatcacttgtcgggcgagtcaggacatttacaattatttagcctggtttcagcagaaaccagggaaagcccctaagtccctgatctatgctgcatccagtttgcaaactggggtcccatcacagttcagcggcagtggatctgggacagatttcactctcaccatcagcagcctccagcctgaagattttgcaacttattactgccaacagtataataattacccattcactttcggccctgggaccaaagtggatatcaaa

SEQ ID NO: 362 - protein, 107 aa, synthetic construct
DIQMTQSPSSLSASVGDRVTITCRASQDIYNYLAWFQQKPGKAPKSLIYAASSLQTGVPSQFSGSGSGTDFTLTISSLQPEDFATYYCQQYNNYPFTFGPGTKVDIK

SEQ ID NO: 363 - DNA, 18 bp, synthetic construct
  source 1..18: mol_type = other DNA
caggacatttacaattat

SEQ ID NO: 364 - protein, 6 aa, synthetic construct
QDIYNY

SEQ ID NO: 365-366 - skipped (2 entries)

SEQ ID NO: 367 - DNA, 27 bp, synthetic construct
  source 1..27: mol_type = other DNA
caacagtataataattacccattcact

SEQ ID NO: 368 - protein, 9 aa, synthetic construct
QQYNNYPFT

SEQ ID NO: 369 - DNA, 360 bp, synthetic construct
  source 1..360: mol_type = other DNA
gaggtgcagctggtggagtctgggggaggcttggcacagcctggggggtccctgagactctcctgtgcagcctctggattcacctttaacaactatgccatgacctgggtccgccaggctccagggaagggtctggactgggtctcagctattagtgatagtggtcgtagcacattctccgcagactccgtgaagggccggttcaccatctccagagacaactccaagaacacgctgtatctgcaaatggacagcctgagagccgaggacacggccttatattactgtgcgaaacataggaactggaactatcccgtctttgactactggggccagggaaccctggtcaccgtctcctca

SEQ ID NO: 370 - protein, 120 aa, synthetic construct
EVQLVESGGGLAQPGGSLRLSCAASGFTFNNYAMTWVRQAPGKGLDWVSAISDSGRSTFSADSVKGRFTISRDNSKNTLYLQMDSLRAEDTALYYCAKHRNWNYPVFDYWGQGTLVTVSS

SEQ ID NO: 371 - DNA, 24 bp, synthetic construct
  source 1..24: mol_type = other DNA
ggattcacctttaacaactatgcc

SEQ ID NO: 372 - protein, 8 aa, synthetic construct
GFTFNNYA

SEQ ID NO: 373 - DNA, 24 bp, synthetic construct
  source 1..24: mol_type = other DNA
attagtgatagtggtcgtagcaca

SEQ ID NO: 374 - protein, 8 aa, synthetic construct
ISDSGRST

SEQ ID NO: 375 - DNA, 39 bp, synthetic construct
  source 1..39: mol_type = other DNA
gcgaaacataggaactggaactatcccgtctttgactac

SEQ ID NO: 376 - protein, 13 aa, synthetic construct
AKHRNWNYPVFDY

SEQ ID NO: 377 - DNA, 321 bp, synthetic construct
  source 1..321: mol_type = other DNA
gacatccagttgacccagtctccatccttcctgtctgcatctgtgggagacagagtcaccatcacttgctgggccagtcagggcattagtagttatttagcctggtatcagcaaaaaccagggaaagccccaaagctcctgatctattctgcatccactttacaaagtggggtcccatcaaggttcagcggcagtggatctgggacagaattcactctcacaatcagcagcctgcagcctgaagattttgcaacttattactgtcaacaacttaatagttacccattcactttcggccctgggaccaaagtggatatcaaa

SEQ ID NO: 378 - protein, 107 aa, synthetic construct
DIQLTQSPSFLSASVGDRVTITCWASQGISSYLAWYQQKPGKAPKLLIYSASTLQSGVPSRFSGSGSGTEFTLTISSLQPEDFATYYCQQLNSYPFTFGPGTKVDIK

SEQ ID NO: 379 - DNA, 18 bp, synthetic construct
  source 1..18: mol_type = other DNA
cagggcattagtagttat

SEQ ID NO: 380 - protein, 6 aa, synthetic construct
QGISSY

SEQ ID NO: 381-382 - skipped (2 entries)

SEQ ID NO: 383 - DNA, 27 bp, synthetic construct
  source 1..27: mol_type = other DNA
caacaacttaatagttacccattcact

SEQ ID NO: 384 - protein, 9 aa, synthetic construct
QQLNSYPFT

SEQ ID NO: 385 - protein, 190 aa, synthetic construct
VKMAETCPIFYDVFFAVANGNELLLDLSLTKVNATEPERTAMKKIQDCYVENGLISRVLDGLVMTTISSSKDCMGEAVQNTVEDLKLNTLGREICPAVKRDVDLFLTGTPDEYVEQVAQYKALPVVLENARILKNCVDAKMTEEDKENALSVLDKIYTSPLCEQKLISEEDLGGEQKLISEEDLHHHHHH

SEQ ID NO: 386-388 - skipped (3 entries)

SEQ ID NO: 389 - protein, 12 aa, synthetic construct
  VARIANT 2: note = Gly, Ser or Asp
  VARIANT 3: note = Ile or Val
  VARIANT 4: note = Ser, Leu, Asn or Gly
  VARIANT 5: note = Asn, Tyr, Gly or Ser
  VARIANT 6: note = Tyr, Ser, Phe or Trp
  VARIANT 7: note = residue may be deleted
  VARIANT 8: note = residue may be deleted
  VARIANT 9: note = residue may be deleted
  VARIANT 10: note = residue may be deleted
  VARIANT 11: note = residue may be deleted
  VARIANT 12: note = residue may be deleted
QXXXXXSNNKQY

SEQ ID NO: 390-391 - skipped (2 entries)

SEQ ID NO: 392 - protein, 70 aa, synthetic construct
EICPAVKRDVDLFLTGTPDEYVEQVAQYKALPVVLENARILKNCVDAKMTEEDKENALSLLDKIYTSPLC

SEQ ID NO: 393 - protein, 92 aa, synthetic construct
VKMAETCPIFYDVFFAVANGNELLLDLSLTKVNATEPERTAMKKIQDCYVENGLISRVLDGLVMTTISSSKDCMGEAVQNTVEDLKLNTLGR

SEQ ID NO: 394 - protein, 398 aa, synthetic construct
VKMAETCPIFYDVFFAVANGNELLLDLSLTKVNATEPERTAMKKIQDCYVENGLISRVLDGLVMTTISSSKDCMGEAVQNTVEDLKLNTLGREICPAVKRDVDLFLTGTPDEYVEQVAQYKALPVVLENARILKNCVDAKMTEEDKENALSLLDKIYTSPLCLINEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 395 - protein, 413 aa, synthetic construct
EICPAVKRDVDLFLTGTPDEYVEQVAQYKALPVVLENARILKNCVDAKMTEEDKENALSLLDKIYTSPLCGGGGSGGGGSGGGGSVKMAETCPIFYDVFFAVANGNELLLDLSLTKVNATEPERTAMKKIQDCYVENGLISRVLDGLVMTTISSSKDCMGEAVQNTVEDLKLNTLGRLINEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 396 - protein, 199 aa, synthetic construct
VKMAETCPIFYDVFFAVANGNELLLDLSLTKVNATEPERTAMKKIQDCYVENGLISRVLDGLVMTTISSSKDCMGEAVQNTVEDLKLNTLGREICPAVKRGVDLFLTGTPDEYVEQVAQYKALPVVLENARILKNCVDAKMTEEDKENALSLLDKIYTSPLCGPGGEQKLISEEDLGGEQKLISEEDLSGHHHHHHSSG

SEQ ID NO: 397 - protein, 214 aa, synthetic construct
EICPAVKRDVDLFLTGTPDEYVEQVAQYKALPVVLENARILKNCVDAKMTEEDKENALSLLDKIYTSPLCGGGGSGGGGSGGGGSVKMAETCPIFYDVFFAVANGNELLLDLSLTKVNATEPERTAMKKIQDCYVENGLISRVLDGLVMTTISSSKDCMGEAVQNTVEDLKLNTLGRGPGGEQKLISEEDLGGEQKLISEEDLSGHHHHHHSSG

SEQ ID NO: 398 - protein, 11 aa, synthetic construct
VKMAETCPIFY

SEQ ID NO: 399 - protein, 7 aa, synthetic construct
ISRVLDG

SEQ ID NO: 400 - protein, 8 aa, synthetic construct
ISRVLDGL

SEQ ID NO: 401 - protein, 10 aa, synthetic construct
ISRVLDGLVM

SEQ ID NO: 402 - protein, 19 aa, synthetic construct
LKLNTLGREICPAVKRGVD

SEQ ID NO: 403 - protein, 20 aa, synthetic construct
LKLNTLGREICPAVKRGVDL

SEQ ID NO: 404 - protein, 15 aa, synthetic construct
YVEQVAQYKALPVVL

SEQ ID NO: 405 - protein, 11 aa, synthetic construct
VAQYKALPVVL

SEQ ID NO: 406 - protein, 14 aa, synthetic construct
ENARILKNCVDAKM

SEQ ID NO: 407 - protein, 18 aa, synthetic construct
LDKIYTSPLCGPGGEQKL

SEQ ID NO: 408 - protein, 14 aa, synthetic construct
ISEEDLSGHHHHHH

SEQ ID NO: 409 - protein, 17 aa, synthetic construct
ISEEDLSGHHHHHHSSG

SEQ ID NO: 410 - protein, 14 aa, synthetic construct
EDLSGHHHHHHSSG

SEQ ID NO: 411 - protein, 11 aa, synthetic construct
VKMAETCPIFY

SEQ ID NO: 412 - protein, 10 aa, synthetic construct
FAVANGNELL

SEQ ID NO: 413 - protein, 7 aa, synthetic construct
ISRVLDG

SEQ ID NO: 414 - protein, 8 aa, synthetic construct
ISRVLDGL

SEQ ID NO: 415 - protein, 10 aa, synthetic construct
ISRVLDGLVM

SEQ ID NO: 416 - protein, 19 aa, synthetic construct
LKLNTLGREICPAVKRGVD

SEQ ID NO: 417 - protein, 20 aa, synthetic construct
LKLNTLGREICPAVKRGVDL

SEQ ID NO: 418 - protein, 15 aa, synthetic construct
YVEQVAQYKALPVVL

SEQ ID NO: 419 - protein, 11 aa, synthetic construct
VAQYKALPVVL

SEQ ID NO: 420 - protein, 8 aa, synthetic construct
YKALPVVL

SEQ ID NO: 421 - protein, 14 aa, synthetic construct
ENARILKNCVDAKM

SEQ ID NO: 422 - protein, 18 aa, synthetic construct
LDKIYTSPLCGPGGEQKL

SEQ ID NO: 423 - protein, 14 aa, synthetic construct
ISEEDLSGHHHHHH

SEQ ID NO: 424 - protein, 17 aa, synthetic construct
ISEEDLSGHHHHHHSSG

SEQ ID NO: 425 - protein, 14 aa, synthetic construct
EDLSGHHHHHHSSG

SEQ ID NO: 426 - protein, 4 aa, synthetic construct
YVEQ

SEQ ID NO: 427 - DNA, 363 bp, synthetic construct
  source 1..363: mol_type = other DNA
gaggtgcagctggtggagtcgggcccaggactggtgaacccttcggagaccctgtccctcacctgctctgtctctggtggctccatcagcagtgttaattactactggggctggatccgccagtccccagggaagggactggagtggattgggagtatctattatactgggagtaccgactacaacccgtccctcaagaatcgagtcaccatatccgtagacacgtccaagaaccagttctccctgaagcagacttctgtgaccgccgcagacacggctgtctattactgtgcgagacatgtggcactggctggggggcttttgatagtctggggccaggggacaatggtcaccgtctcttca

SEQ ID NO: 428 - protein, 121 aa, synthetic construct
EVQLVESGPGLVNPSETLSLTCSVSGGSISSVNYYWGWIRQSPGKGLEWIGSIYYTGSTDYNPSLKNRVTISVDTSKNQFSLKQTSVTAADTAVYYCARHVALAGGLLIVWGQGTMVTVSS

SEQ ID NO: 429 - DNA, 30 bp, synthetic construct; identical to SEQ ID NO: 179
  source 1..30: mol_type = other DNA

SEQ ID NO: 430 - protein, 10 aa, synthetic construct
GGSISSVNYY

SEQ ID NO: 431 - DNA, 21 bp, synthetic construct
  source 1..21: mol_type = other DNA
atctattatactgggagtacc

SEQ ID NO: 432 - protein, 7 aa, synthetic construct
IYYTGST

SEQ ID NO: 433 - DNA, 39 bp, synthetic construct
  source 1..39: mol_type = other DNA
gcgagacatgtggcactggctggggggcttttgatagtc

SEQ ID NO: 434 - protein, 13 aa, synthetic construct
ARHVALAGGLLIV

SEQ ID NO: 435 - DNA, 342 bp, synthetic construct
  source 1..342: mol_type = other DNA
gacatccagatgacccagtctccagactccctggctgtgtctctgggcgcgagggccaccatcaactgcaagtccagccaaagtgttttattcagctccaacaataagaacttcttagcctggtaccagcagaaaccaggacagcctcctaccctgctcatttcctgggcatctacccgggaatccggggtccctgaccgattcagtgggagcgggtctgggacagatttcactctcaccatcagcagcctgcaggctgaagatgtggcagtttatttctgtcaacaatattataatagtcctccacttttcggccaggggaccaaggtggagatcaaacga

SEQ ID NO: 436 - protein, 114 aa, synthetic construct
DIQMTQSPDSLAVSLGARATINCKSSQSVLFSSNNKNFLAWYQQKPGQPPTLLISWASTRESGVPDRFSGSGSGTDFTLTISSLQAEDVAVYFCQQYYNSPPLFGQGTKVEIKR

SEQ ID NO: 437 - DNA, 36 bp, synthetic construct
  source 1..36: mol_type = other DNA
caaagtgttttattcagctccaacaataagaacttc

SEQ ID NO: 438 - protein, 12 aa, synthetic construct
QSVLFSSNNKNF

SEQ ID NO: 439-440 - skipped (2 entries)

SEQ ID NO: 441 - DNA, 27 bp, synthetic construct
  source 1..27: mol_type = other DNA
caacaatattataatagtcctccactt

SEQ ID NO: 442 - protein, 9 aa, synthetic construct
QQYYNSPPL

SEQ ID NO: 443 - DNA, 360 bp, synthetic construct
  source 1..360: mol_type = other DNA
gaggtgcagctggtggagtctgggggaggcttggtacagcctggggggtccctgagactctcctgtgcagtctctggattcacctttagcaactatgccatgaactgggtccgccaggctccagggaaggggctggagtgggtctcagttattagcgacagtggtcgtagcacctactacgcagactccgtgaagggccggttcaccatctccagagacaattccaagaacacgctatatctgcaaatgaacagcctgagagccgaggacacggccgtatatttctgtgcgaaaaggtataactggaacttacactactttgactactggggccagggaaccacggtcaccgtctcctca

SEQ ID NO: 444 - protein, 120 aa, synthetic construct
EVQLVESGGGLVQPGGSLRLSCAVSGFTFSNYAMNWVRQAPGKGLEWVSVISDSGRSTYYADSVKGRFTISRDNSKNTLYLQMNSLRAEDTAVYFCAKRYNWNLHYFDYWGQGTTVTVSS

SEQ ID NO: 445 - DNA, 24 bp, synthetic construct
  source 1..24: mol_type = other DNA
ggattcacctttagcaactatgcc

SEQ ID NO: 446 - protein, 8 aa, synthetic construct
GFTFSNYA

SEQ ID NO: 447 - DNA, 24 bp, synthetic construct
  source 1..24: mol_type = other DNA
attagcgacagtggtcgtagcacc

SEQ ID NO: 448 - protein, 8 aa, synthetic construct
ISDSGRST

SEQ ID NO: 449 - DNA, 39 bp, synthetic construct
  source 1..39: mol_type = other DNA
gcgaaaaggtataactggaacttacactactttgactac

SEQ ID NO: 450 - protein, 13 aa, synthetic construct
AKRYNWNLHYFDY

SEQ ID NO: 451 - DNA, 321 bp, synthetic construct
  source 1..321: mol_type = other DNA
gaaattgtgttgacgcagtctccagactttcagtctgtgactccaaaggagaaagtcaccatcacctgccgggccagtcagagcattggtggtagcttacactggtaccagcagaaaccagatcagtctccaaagctcctcatcaagtatgcttcccagtccttctcaggggtcccctcgaggttcagtggcagtggatctgggacagatttcaccctcaccatcaatagcctggaagctgaagatgctgcaacgtattactgtcttcagagtagtagtttacggacgttcggccaagggaccaaggtggagatcaaacga

SEQ ID NO: 452 - protein, 107 aa, synthetic construct
EIVLTQSPDFQSVTPKEKVTITCRASQSIGGSLHWYQQKPDQSPKLLIKYASQSFSGVPSRFSGSGSGTDFTLTINSLEAEDAATYYCLQSSSLRTFGQGTKVEIKR

SEQ ID NO: 453 - DNA, 18 bp, synthetic construct
  source 1..18: mol_type = other DNA
cagagcattggtggtagc

SEQ ID NO: 454 - protein, 6 aa, synthetic construct
QSIGGS

SEQ ID NO: 455-456 - skipped (2 entries)

SEQ ID NO: 457 - DNA, 24 bp, synthetic construct
  source 1..24: mol_type = other DNA
cttcagagtagtagtttacggacg

SEQ ID NO: 458 - protein, 8 aa, synthetic construct
LQSSSLRT

SEQ ID NO: 459 - DNA, 345 bp, synthetic construct
  source 1..345: mol_type = other DNA
gaggtgcagctggtggagtctgggggaggcttggtacagcctggggggtccctgagactctcctgtgcagcctctggattcacctttagcagttatgccatgagctgggtccgccaggctccagggaaggggctggagtgggtctcagctattagtggtcgtggttataacgcagactacgcagactccgtgaagggccggttcaccatctccagagacaattccaagaacacgctgtatctgcaaatgaacagcctgagagccgaagacacggccgtatattactgtgcgaaattggaatactttgactactggggccagggaaccacggtcaccgtctcctca

SEQ ID NO: 460 - protein, 115 aa, synthetic construct
EVQLVESGGGLVQPGGSLRLSCAASGFTFSSYAMSWVRQAPGKGLEWVSAISGRGYNADYADSVKGRFTISRDNSKNTLYLQMNSLRAEDTAVYYCAKLEYFDYWGQGTTVTVSS

SEQ ID NO: 461 - DNA, 24 bp, synthetic construct
  source 1..24: mol_type = other DNA
ggattcacctttagcagttatgcc

SEQ ID NO: 462 - protein, 8 aa, synthetic construct
GFTFSSYA

SEQ ID NO: 463 - DNA, 24 bp, synthetic construct
  source 1..24: mol_type = other DNA
attagtggtcgtggttataacgca

SEQ ID NO: 464 - protein, 8 aa, synthetic construct
ISGRGYNA

SEQ ID NO: 465 - DNA, 24 bp, synthetic construct
  source 1..24: mol_type = other DNA
gcgaaattggaatactttgactac

SEQ ID NO: 466 - protein, 8 aa, synthetic construct
AKLEYFDY

SEQ ID NO: 467 - DNA, 324 bp, synthetic construct
  source 1..324: mol_type = other DNA
gacatccagttgacccagtctccttccaccctgtctgcatctgtaggagacagagtcaccatcacttgccgggccagtcagagtattagtagctggttggcctggtatcagcagaaaccagggaaagcccctaagctcctgatctataaggcgtctagtttagaaagtggggtcccatcaaggttcagcggcagtggatctgggacagatttcactctcaccatcagcagcctgaggcctgaagattttgcaacttattactgccaacagtataatagttaccctctgactttcggcggagggaccaaggtggaaatcaaacga

SEQ ID NO: 468 - protein, 108 aa, synthetic construct
DIQLTQSPSTLSASVGDRVTITCRASQSISSWLAWYQQKPGKAPKLLIYKASSLESGVPSRFSGSGSGTDFTLTISSLRPEDFATYYCQQYNSYPLTFGGGTKVEIKR

SEQ ID NO: 469 - DNA, 18 bp, synthetic construct
  source 1..18: mol_type = other DNA
cagagtattagtagctgg

SEQ ID NO: 470 - protein, 6 aa, synthetic construct
QSISSW

SEQ ID NO: 471-472 - skipped (2 entries)

SEQ ID NO: 473 - DNA, 27 bp, synthetic construct
  source 1..27: mol_type = other DNA
caacagtataatagttaccctctgact

SEQ ID NO: 474 - protein, 9 aa, synthetic construct
QQYNSYPLT

SEQ ID NO: 475 - DNA, 345 bp, synthetic construct
  source 1..345: mol_type = other DNA
gaggtgcagctggtggagtctgggggaggcttggtacagcctggggggtccctgagactctcctgtgcagcctctggattcacctttagcacctatgccatgcactgggtccgccaggctccagggaaggggctggagtgggtctcaagtattagtggtcgtggtcgtaactcagaccacgcagactccgtgaagggccggttcaccatctccagagacaattccaagaacacgctctatctacaaatgaacagcctgagagccgaggacacggccgtatattactgtgcgaggaccgaatacttccaccactggggccagggcaccacggtcaccgtctcctca

SEQ ID NO: 476 - protein, 115 aa, synthetic construct
EVQLVESGGGLVQPGGSLRLSCAASGFTFSTYAMHWVRQAPGKGLEWVSSISGRGRNSDHADSVKGRFTISRDNSKNTLYLQMNSLRAEDTAVYYCARTEYFHHWGQGTTVTVSS

SEQ ID NO: 477 - DNA, 24 bp, synthetic construct
  source 1..24: mol_type = other DNA
ggattcacctttagcacctatgcc

SEQ ID NO: 478 - protein, 8 aa, synthetic construct
GFTFSTYA

SEQ ID NO: 479 - DNA, 24 bp, synthetic construct
  source 1..24: mol_type = other DNA
attagtggtcgtggtcgtaactca

SEQ ID NO: 480 - protein, 8 aa, synthetic construct
ISGRGRNS

SEQ ID NO: 481 - DNA, 24 bp, synthetic construct
  source 1..24: mol_type = other DNA
gcgaggaccgaatacttccaccac

SEQ ID NO: 482 - protein, 8 aa, synthetic construct
ARTEYFHH

SEQ ID NO: 483 - DNA, 324 bp, synthetic construct
  source 1..324: mol_type = other DNA
gacatcgtgatgacccagtctccatcctcactgtctgcatctgtaggagacagaatcaccatcacttgtcgggcgagtcaggacattaacaattatttagcctggtttcagcagaaaccaggaaaagcccctaagtccctgatctatggtgcatccagcttgcaaagtggggtcccatcaaagttcagcggcagtggatctgggacagatttcactctcaccatcagcagcctgcagcctgaagattttgcaacttattactgccaacaatatacgttcttaccattcactttcggccctgggaccaaggtgaaaatcaaacga

SEQ ID NO: 484 - protein, 108 aa, synthetic construct
DIVMTQSPSSLSASVGDRITITCRASQDINNYLAWFQQKPGKAPKSLIYGASSLQSGVPSKFSGSGSGTDFTLTISSLQPEDFATYYCQQYTFLPFTFGPGTKVKIKR

SEQ ID NO: 485 - DNA, 18 bp, synthetic construct
  source 1..18: mol_type = other DNA
caggacattaacaattat

SEQ ID NO: 486 - protein, 6 aa, synthetic construct
QDINNY

SEQ ID NO: 487-488 - skipped (2 entries)

SEQ ID NO: 489 - DNA, 27 bp, synthetic construct
  source 1..27: mol_type = other DNA
caacaatatacgttcttaccattcact

SEQ ID NO: 490 - protein, 9 aa, synthetic construct
QQYTFLPFT

SEQ ID NO: 491 - protein, 15 aa, synthetic construct
GGGGSGGGGSGGGGS